SEQUENCE LISTING (507 sequences)

SEQ ID NO: 1 - DNA, 38 bp, Artificial Sequence
  siRNA target
tgacagcagtgttgataaatgacagcagtgttgataaa

SEQ ID NO: 2 - DNA, 19 bp, Artificial Sequence
  siRNA target
tgacagcagtgttgataaa

SEQ ID NO: 3 - DNA, 19 bp, Artificial Sequence
  siRNA target
aagaacgagtgctcaataa

SEQ ID NO: 4 - DNA, 19 bp, Artificial Sequence
  siRNA target
tttatgaactgacgttaca

SEQ ID NO: 5 - DNA, 19 bp, Artificial Sequence
  siRNA target
ggagtattgtggaacttat

SEQ ID NO: 6 - DNA, 19 bp, Artificial Sequence
  siRNA target
gagtattgtggaacttata

SEQ ID NO: 7 - DNA, 19 bp, Artificial Sequence
  siRNA target
agaccgtgtgaatcattgt

SEQ ID NO: 8 - DNA, 19 bp, Artificial Sequence
  siRNA target
ggttacagctcgagctcta

SEQ ID NO: 9 - DNA, 19 bp, Artificial Sequence
  siRNA target
ggttttgttaaaggccttc

SEQ ID NO: 10 - DNA, 27 bp, Artificial Sequence
  siRNA target
tgacagcagtgttgataaatttgtgtt

SEQ ID NO: 11 - DNA, 27 bp, Artificial Sequence
  siRNA target
aagaacgagtgctcaataatgttgtca

SEQ ID NO: 12 - DNA, 27 bp, Artificial Sequence
  siRNA target
tttatgaactgacgttacatcatacac

SEQ ID NO: 13 - DNA, 27 bp, Artificial Sequence
  siRNA target
ggagtattgtggaacttatagctggag

SEQ ID NO: 14 - DNA, 27 bp, Artificial Sequence
  siRNA target
gagtattgtggaacttatagctggagg

SEQ ID NO: 15 - DNA, 27 bp, Artificial Sequence
  siRNA target
agaccgtgtgaatcattgtctgacaat

SEQ ID NO: 16 - DNA, 27 bp, Artificial Sequence
  siRNA target
ggttttgttaaaggccttcatagcgaa

SEQ ID NO: 17 - DNA, 21 bp, Artificial Sequence
  siRNA target
aagggtgtgtatgtgccctac

SEQ ID NO: 18 - DNA, 21 bp, Artificial Sequence
  siRNA target
aattggctttgctgtcagcgc

SEQ ID NO: 19 - DNA, 21 bp, Artificial Sequence
  siRNA target
aagactgtggctacaacattc

SEQ ID NO: 20 - DNA, 21 bp, Artificial Sequence
  siRNA target
aaggctgcctggagaaaggat

SEQ ID NO: 21 - DNA, 21 bp, Artificial Sequence
  siRNA target
cactgaatcggacaagttctt

SEQ ID NO: 22 - DNA, 21 bp, Artificial Sequence
  siRNA target
catgatcattggtggtatcga

SEQ ID NO: 23 - DNA, 21 bp, Artificial Sequence
  siRNA target
catccttcctcagcaatacct

SEQ ID NO: 24 - DNA, 21 bp, Artificial Sequence
  siRNA target
cagacgctcaacatcctggtg

SEQ ID NO: 25 - DNA, 19 bp, Artificial Sequence
  siRNA target
ctacgaacctgaagcctaa

SEQ ID NO: 26 - DNA, 19 bp, Artificial Sequence
  siRNA target
tcaagactacgaacctgaa

SEQ ID NO: 27 - DNA, 19 bp, Artificial Sequence
  siRNA target
cattagccatggatgtatt

SEQ ID NO: 28 - DNA, 19 bp, Artificial Sequence
  siRNA target
acgaacctgaagcctaaga

SEQ ID NO: 29 - DNA, 19 bp, Artificial Sequence
  siRNA target
gtacaagtgctcagttcca

SEQ ID NO: 30 - DNA, 19 bp, Artificial Sequence
  siRNA target
gcttcaatctacgatgtta

SEQ ID NO: 31 - DNA, 19 bp, Artificial Sequence
  siRNA target
ctaagtgactaccacttat

SEQ ID NO: 32 - DNA, 19 bp, Artificial Sequence
  siRNA target
gttcagaagttgttagtga

SEQ ID NO: 33 - DNA, 19 bp, Artificial Sequence
  siRNA target
agttgttagtgatttgcta

SEQ ID NO: 34 - DNA, 19 bp, Artificial Sequence
  siRNA target
gacgtattgtgaaatttgt

SEQ ID NO: 35 - DNA, 19 bp, Artificial Sequence
  siRNA target
tcatcaatttcgagcagaa

SEQ ID NO: 36 - DNA, 19 bp, Artificial Sequence
  siRNA target
tgagtttggagataataca

SEQ ID NO: 37 - DNA, 19 bp, Artificial Sequence
  siRNA target
tggccgatgtgtctattga

SEQ ID NO: 38 - DNA, 19 bp, Artificial Sequence
  siRNA target
cgatgtgtctattgaagat

SEQ ID NO: 39 - DNA, 19 bp, Artificial Sequence
  siRNA target
gcattaaaggactgactga

SEQ ID NO: 40 - DNA, 19 bp, Artificial Sequence
  siRNA target
tcgtttggcttgtggtgta

SEQ ID NO: 41 - DNA, 21 bp, Artificial Sequence
  siRNA target
aatttcgagcagaaggaaagt

SEQ ID NO: 42 - DNA, 21 bp, Artificial Sequence
  siRNA target
aagcattaaaggactgactga

SEQ ID NO: 43 - DNA, 21 bp, Artificial Sequence
  siRNA target
aatgtgactgctgacaaagat

SEQ ID NO: 44 - DNA, 21 bp, Artificial Sequence
  siRNA target
aagattctgtgatctcactct

SEQ ID NO: 45 - DNA, 19 bp, Artificial Sequence
  siRNA target
ccagggacctctctctaat

SEQ ID NO: 46 - DNA, 19 bp, Artificial Sequence
  siRNA target
agggacctctctctaatca

SEQ ID NO: 47 - DNA, 19 bp, Artificial Sequence
  siRNA target
gcctgtagcccatgttgta

SEQ ID NO: 48 - DNA, 19 bp, Artificial Sequence
  siRNA target
tgtagcccatgttgtagca

SEQ ID NO: 49 - DNA, 19 bp, Artificial Sequence
  siRNA target
gtgaaatgatggcttatta

SEQ ID NO: 50 - DNA, 19 bp, Artificial Sequence
  siRNA target
cgatgcacctgtacgatca

SEQ ID NO: 51 - DNA, 19 bp, Artificial Sequence
  siRNA target
taactgacttcaccatgca

SEQ ID NO: 52 - DNA, 19 bp, Artificial Sequence
  siRNA target
gaacctatcttcttcgaca

SEQ ID NO: 53 - DNA, 19 bp, Artificial Sequence
  siRNA target
cagaagaacagaagaaata

SEQ ID NO: 54 - DNA, 19 bp, Artificial Sequence
  siRNA target
tgaagaagctaaacagaaa

SEQ ID NO: 55 - DNA, 19 bp, Artificial Sequence
  siRNA target
ggtaagagctacaaagaat

SEQ ID NO: 56 - DNA, 19 bp, Artificial Sequence
  siRNA target
aggcagaggaagagatata

SEQ ID NO: 57 - DNA, 19 bp, Artificial Sequence
  siRNA target
agacagagatgatgattta

SEQ ID NO: 58 - DNA, 19 bp, Artificial Sequence
  siRNA target
gggaaagacagcaaggaaa

SEQ ID NO: 59 - DNA, 19 bp, Artificial Sequence
  siRNA target
gaacaagacagaacagaaa

SEQ ID NO: 60 - DNA, 19 bp, Artificial Sequence
  siRNA target
gtgaagaagactttagaaa

SEQ ID NO: 61 - DNA, 19 bp, Artificial Sequence
  siRNA target
ccaaagatttccagggaga

SEQ ID NO: 62 - DNA, 19 bp, Artificial Sequence
  siRNA target
taacatagagtcagggaaa

SEQ ID NO: 63 - DNA, 19 bp, Artificial Sequence
  siRNA target
gaaagaagaaacagaagaa

SEQ ID NO: 64 - DNA, 19 bp, Artificial Sequence
  siRNA target
ggagataagacaagcagaa

SEQ ID NO: 65 - DNA, 19 bp, Artificial Sequence
  siRNA target
ctgaatactaagaaggaaa

SEQ ID NO: 66 - DNA, 19 bp, Artificial Sequence
  siRNA target
gagaagaagcagaggctga

SEQ ID NO: 67 - DNA, 19 bp, Artificial Sequence
  siRNA target
gaaagatgatgatgaagaa

SEQ ID NO: 68 - DNA, 19 bp, Artificial Sequence
  siRNA target
tgggaaacctgaagcataa

SEQ ID NO: 69 - DNA, 19 bp, Artificial Sequence
  siRNA target
gaacacagttggtttgaaa

SEQ ID NO: 70 - DNA, 19 bp, Artificial Sequence
  siRNA target
tgacagaagaacagaagaa

SEQ ID NO: 71 - DNA, 19 bp, Artificial Sequence
  siRNA target
aagaagaagctgaggcaat

SEQ ID NO: 72 - DNA, 19 bp, Artificial Sequence
  siRNA target
tttcaaaggcagaggaaga

SEQ ID NO: 73 - DNA, 19 bp, Artificial Sequence
  siRNA target
cttgaagagtccagacaaa

SEQ ID NO: 74 - DNA, 19 bp, Artificial Sequence
  siRNA target
gctaaagaaagaagaaaca

SEQ ID NO: 75 - DNA, 19 bp, Artificial Sequence
  siRNA target
agaagaaacagaagaaaga

SEQ ID NO: 76 - DNA, 19 bp, Artificial Sequence
  siRNA target
gctgagaaattgtcgaaat

SEQ ID NO: 77 - DNA, 19 bp, Artificial Sequence
  siRNA target
gagcaagcatattaacaaa

SEQ ID NO: 78 - DNA, 19 bp, Artificial Sequence
  siRNA target
cataaaagatggagacaga

SEQ ID NO: 79 - DNA, 19 bp, Artificial Sequence
  siRNA target
taacaaagccagacaaaga

SEQ ID NO: 80 - DNA, 19 bp, Artificial Sequence
  siRNA target
aaaggaagacaaagggaaa

SEQ ID NO: 81 - DNA, 19 bp, Artificial Sequence
  siRNA target
aaagggagatgctgagaaa

SEQ ID NO: 82 - DNA, 19 bp, Artificial Sequence
  siRNA target
taacaaacactgtggaaga

SEQ ID NO: 83 - DNA, 19 bp, Artificial Sequence
  siRNA target
agtattgaacaaagggaaa

SEQ ID NO: 84 - DNA, 19 bp, Artificial Sequence
  siRNA target
aggcgaagcagcagaacaa

SEQ ID NO: 85 - DNA, 19 bp, Artificial Sequence
  siRNA target
tagcagatgtggaaggatt

SEQ ID NO: 86 - DNA, 19 bp, Artificial Sequence
  siRNA target
aaacaaaccttacgtgaat

SEQ ID NO: 87 - DNA, 19 bp, Artificial Sequence
  siRNA target
aaatatgaatgctgaggaa

SEQ ID NO: 88 - DNA, 19 bp, Artificial Sequence
  siRNA target
ccaaagaagaaaagaaaga

SEQ ID NO: 89 - DNA, 19 bp, Artificial Sequence
  siRNA target
ctgacaaactgcatattta

SEQ ID NO: 90 - DNA, 19 bp, Artificial Sequence
  siRNA target
agggagatgctgagaaatt

SEQ ID NO: 91 - DNA, 19 bp, Artificial Sequence
  siRNA target
cattgaacatgctgattaa

SEQ ID NO: 92 - DNA, 19 bp, Artificial Sequence
  siRNA target
gcatgcagctctttggtaa

SEQ ID NO: 93 - DNA, 19 bp, Artificial Sequence
  siRNA target
agacaatcttacagcaatt

SEQ ID NO: 94 - DNA, 19 bp, Artificial Sequence
  siRNA target
aagaagaccctgatgcaaa

SEQ ID NO: 95 - DNA, 19 bp, Artificial Sequence
  siRNA target
ggaagacagtgatggtcaa

SEQ ID NO: 96 - DNA, 19 bp, Artificial Sequence
  siRNA target
cagacaagatcttcactta

SEQ ID NO: 97 - DNA, 19 bp, Artificial Sequence
  siRNA target
agccagacaaagagaaata

SEQ ID NO: 98 - DNA, 19 bp, Artificial Sequence
  siRNA target
cttcgaactttcagagtat

SEQ ID NO: 99 - DNA, 19 bp, Artificial Sequence
  siRNA target
gagtagagcaagcatatta

SEQ ID NO: 100 - DNA, 19 bp, Artificial Sequence
  siRNA target
tgtacttgctataggaaat

SEQ ID NO: 101 - DNA, 19 bp, Artificial Sequence
  siRNA target
ggtcaagctatgtgcctta

SEQ ID NO: 102 - DNA, 19 bp, Artificial Sequence
  siRNA target
gaaacaaaccttacgtgaa

SEQ ID NO: 103 - DNA, 19 bp, Artificial Sequence
  siRNA target
gattatggctacacgagct

SEQ ID NO: 104 - DNA, 19 bp, Artificial Sequence
  siRNA target
gatggattctcttcgttca

SEQ ID NO: 105 - DNA, 19 bp, Artificial Sequence
  siRNA target
tgtttcagctcttcgaact

SEQ ID NO: 106 - DNA, 19 bp, Artificial Sequence
  siRNA target
ccgacattgtggacttaca

SEQ ID NO: 107 - DNA, 19 bp, Artificial Sequence
  siRNA target
gcttagataccttcatgaa

SEQ ID NO: 108 - DNA, 19 bp, Artificial Sequence
  siRNA target
gggaacaagaccagagttt

SEQ ID NO: 109 - DNA, 19 bp, Artificial Sequence
  siRNA target
agattgacctggagaagta

SEQ ID NO: 110 - DNA, 19 bp, Artificial Sequence
  siRNA target
ctgctggcctggagggaaa

SEQ ID NO: 111 - DNA, 19 bp, Artificial Sequence
  siRNA target
gcttaatgaatgaggatga

SEQ ID NO: 112 - DNA, 19 bp, Artificial Sequence
  siRNA target
cagcagaagtacacagtga

SEQ ID NO: 113 - DNA, 19 bp, Artificial Sequence
  siRNA target
ggacattgttgttagcgaa

SEQ ID NO: 114 - DNA, 19 bp, Artificial Sequence
  siRNA target
acttaaagctggttcatat

SEQ ID NO: 115 - DNA, 19 bp, Artificial Sequence
  siRNA target
tgacagaggatgagagtct

SEQ ID NO: 116 - DNA, 19 bp, Artificial Sequence
  siRNA target
ctgcagagcttgaaggcca

SEQ ID NO: 117 - DNA, 19 bp, Artificial Sequence
  siRNA target
gagctgtacaggagactaa

SEQ ID NO: 118 - DNA, 19 bp, Artificial Sequence
  siRNA target
gagaagaaagtgcgagtga

SEQ ID NO: 119 - DNA, 19 bp, Artificial Sequence
  siRNA target
ggagaagtacagcgagcaa

SEQ ID NO: 120 - DNA, 19 bp, Artificial Sequence
  siRNA target
agaaagtgcgagtgatcta

SEQ ID NO: 121 - DNA, 19 bp, Artificial Sequence
  siRNA target
gaaagagcgccttgggaca

SEQ ID NO: 122 - DNA, 19 bp, Artificial Sequence
  siRNA target
gcttcaaggccctggatga

SEQ ID NO: 123 - DNA, 19 bp, Artificial Sequence
  siRNA target
ggttacagacggaagaaga

SEQ ID NO: 124 - DNA, 19 bp, Artificial Sequence
  siRNA target
tgatgaatctcctccgaaa

SEQ ID NO: 125 - DNA, 19 bp, Artificial Sequence
  siRNA target
aagtgaaactcctggtaga

SEQ ID NO: 126 - DNA, 19 bp, Artificial Sequence
  siRNA target
ggaaacaggtgagcagatt

SEQ ID NO: 127 - DNA, 19 bp, Artificial Sequence
  siRNA target
gcaagttaaatgagggcca

SEQ ID NO: 128 - DNA, 19 bp, Artificial Sequence
  siRNA target
gaagaaagtgcgagtgatc

SEQ ID NO: 129 - DNA, 19 bp, Artificial Sequence
  siRNA target
atgaatgcctctcgactta

SEQ ID NO: 130 - DNA, 19 bp, Artificial Sequence
  siRNA target
gggcctgggaaatgaaaga

SEQ ID NO: 131 - DNA, 19 bp, Artificial Sequence
  siRNA target
gcgaagacttgaatggaac

SEQ ID NO: 132 - DNA, 19 bp, Artificial Sequence
  siRNA target
ccaataatcttaacagtgt

SEQ ID NO: 133 - DNA, 19 bp, Artificial Sequence
  siRNA target
ttcaagagcccaagaggaa

SEQ ID NO: 134 - DNA, 19 bp, Artificial Sequence
  siRNA target
gggaaatggagcaggctgt

SEQ ID NO: 135 - DNA, 19 bp, Artificial Sequence
  siRNA target
agaccgacattgtggactt

SEQ ID NO: 136 - DNA, 19 bp, Artificial Sequence
  siRNA target
tacaggagactaagggaaa

SEQ ID NO: 137 - DNA, 19 bp, Artificial Sequence
  siRNA target
gaagaggtggtgagcttaa

SEQ ID NO: 138 - DNA, 19 bp, Artificial Sequence
  siRNA target
gggaaatgaaagagcgcct

SEQ ID NO: 139 - DNA, 19 bp, Artificial Sequence
  siRNA target
acacagtgaccgtcgacta

SEQ ID NO: 140 - DNA, 19 bp, Artificial Sequence
  siRNA target
atgaagaattccatggctt

SEQ ID NO: 141 - DNA, 19 bp, Artificial Sequence
  siRNA target
gttacagacggaagaagaa

SEQ ID NO: 142 - DNA, 19 bp, Artificial Sequence
  siRNA target
ggaagtacctgaaccagtt

SEQ ID NO: 143 - DNA, 19 bp, Artificial Sequence
  siRNA target
tgggatcacatcagataaa

SEQ ID NO: 144 - DNA, 19 bp, Artificial Sequence
  siRNA target
gagcttaatgaatgaggat

SEQ ID NO: 145 - DNA, 19 bp, Artificial Sequence
  siRNA target
ccaagaagagtgaagaact

SEQ ID NO: 146 - DNA, 19 bp, Artificial Sequence
  siRNA target
tgacattgcctctgcgctt

SEQ ID NO: 147 - DNA, 19 bp, Artificial Sequence
  siRNA target
tggctgagcgactggagaa

SEQ ID NO: 148 - DNA, 19 bp, Artificial Sequence
  siRNA target
tttcagacggcaagttaaa

SEQ ID NO: 149 - DNA, 19 bp, Artificial Sequence
  siRNA target
tggacgacctagaggagca

SEQ ID NO: 150 - DNA, 19 bp, Artificial Sequence
  siRNA target
taatgaatgaggatgagaa

SEQ ID NO: 151 - DNA, 19 bp, Artificial Sequence
  siRNA target
agccaagaagagtgaagaa

SEQ ID NO: 152 - DNA, 19 bp, Artificial Sequence
  siRNA target
caagaagagtgaagaactg

SEQ ID NO: 153 - DNA, 19 bp, Artificial Sequence
  siRNA target
agaagagtgaggtggacat

SEQ ID NO: 154 - DNA, 19 bp, Artificial Sequence
  siRNA target
agcgaagacttgaatggaa

SEQ ID NO: 155 - DNA, 19 bp, Artificial Sequence
  siRNA target
aggaaacaggtgagcagat

SEQ ID NO: 156 - DNA, 19 bp, Artificial Sequence
  siRNA target
cttagataccttcatgaaa

SEQ ID NO: 157 - DNA, 19 bp, Artificial Sequence
  siRNA target
gcatccagaccaacaacaa

SEQ ID NO: 158 - DNA, 19 bp, Artificial Sequence
  siRNA target
agcgcatccagaccaacaa

SEQ ID NO: 159 - DNA, 19 bp, Artificial Sequence
  siRNA target
gtgacaaggtgcagaaaga

SEQ ID NO: 160 - DNA, 19 bp, Artificial Sequence
  siRNA target
ggattgaggagaaacgtaa

SEQ ID NO: 161 - DNA, 19 bp, Artificial Sequence
  siRNA target
cccacgagcttgtaggaaa

SEQ ID NO: 162 - DNA, 19 bp, Artificial Sequence
  siRNA target
ggagaaacgtaaaaggaca

SEQ ID NO: 163 - DNA, 19 bp, Artificial Sequence
  siRNA target
ggcgagaggagcacagata

SEQ ID NO: 164 - DNA, 19 bp, Artificial Sequence
  siRNA target
ctacacaggaccagggaca

SEQ ID NO: 165 - DNA, 19 bp, Artificial Sequence
  siRNA target
aagaggacattgaggtgta

SEQ ID NO: 166 - DNA, 19 bp, Artificial Sequence
  siRNA target
ggaaaggactgccgggatg

SEQ ID NO: 167 - DNA, 19 bp, Artificial Sequence
  siRNA target
tcaagagcatcatgaagaa

SEQ ID NO: 168 - DNA, 19 bp, Artificial Sequence
  siRNA target
tggagtaccctgaggctat

SEQ ID NO: 169 - DNA, 19 bp, Artificial Sequence
  siRNA target
gaatccagtgtgtgaagaa

SEQ ID NO: 170 - DNA, 19 bp, Artificial Sequence
  siRNA target
tcagtgagcccatggaatt

SEQ ID NO: 171 - DNA, 19 bp, Artificial Sequence
  siRNA target
cgtaaaaggacatatgaga

SEQ ID NO: 172 - DNA, 19 bp, Artificial Sequence
  siRNA target
gggaaggaacgctgtcaga

SEQ ID NO: 173 - DNA, 19 bp, Artificial Sequence
  siRNA target
ccacgagcttgtaggaaag

SEQ ID NO: 174 - DNA, 19 bp, Artificial Sequence
  siRNA target
cttccaagttcctatagaa

SEQ ID NO: 175 - DNA, 19 bp, Artificial Sequence
  siRNA target
gcatccagaccaacaacaa

SEQ ID NO: 176 - DNA, 19 bp, Artificial Sequence
  siRNA target
ctcaagatctgccgagtga

SEQ ID NO: 177 - DNA, 19 bp, Artificial Sequence
  siRNA target
cggattgaggagaaacgta

SEQ ID NO: 178 - DNA, 19 bp, Artificial Sequence
  siRNA target
gattgaggagaaacgtaaa

SEQ ID NO: 179 - DNA, 19 bp, Artificial Sequence
  siRNA target
ggacatatgagaccttcaa

SEQ ID NO: 180 - DNA, 19 bp, Artificial Sequence
  siRNA target
tcaccggattgaggagaaa

SEQ ID NO: 181 - DNA, 19 bp, Artificial Sequence
  siRNA target
caactgagcccatgctgat

SEQ ID NO: 182 - DNA, 19 bp, Artificial Sequence
  siRNA target
aggaaaggactgccgggat

SEQ ID NO: 183 - DNA, 19 bp, Artificial Sequence
  siRNA target
ccaacactgccgagctcaa

SEQ ID NO: 184 - DNA, 19 bp, Artificial Sequence
  siRNA target
gctgcagtttgatgatgaa

SEQ ID NO: 185 - DNA, 19 bp, Artificial Sequence
  siRNA target
gaccagggacagtgcgcat

SEQ ID NO: 186 - DNA, 19 bp, Artificial Sequence
  siRNA target
gggatgagatcttcctact

SEQ ID NO: 187 - DNA, 19 bp, Artificial Sequence
  siRNA target
aggtgcagaaagaggacat

SEQ ID NO: 188 - DNA, 19 bp, Artificial Sequence
  siRNA target
aggacattgaggtgtattt

SEQ ID NO: 189 - DNA, 19 bp, Artificial Sequence
  siRNA target
gagaaacgtaaaaggacat

SEQ ID NO: 190 - DNA, 19 bp, Artificial Sequence
  siRNA target
catcaagatcaatggctac

SEQ ID NO: 191 - DNA, 19 bp, Artificial Sequence
  siRNA target
ccaagttcctatagaagag

SEQ ID NO: 192 - DNA, 19 bp, Artificial Sequence
  siRNA target
agatcttcctactgtgtga

SEQ ID NO: 193 - DNA, 19 bp, Artificial Sequence
  siRNA target
agaaagaggacattgaggt

SEQ ID NO: 194 - DNA, 19 bp, Artificial Sequence
  siRNA target
gtcaccggattgaggagaa

SEQ ID NO: 195 - DNA, 19 bp, Artificial Sequence
  siRNA target
acatatgagaccttcaaga

SEQ ID NO: 196 - DNA, 19 bp, Artificial Sequence
  siRNA target
gctataactcgcctagtga

SEQ ID NO: 197 - DNA, 19 bp, Artificial Sequence
  siRNA target
tgcagaaagaggacattga

SEQ ID NO: 198 - DNA, 19 bp, Artificial Sequence
  siRNA target
gaaagaggacattgaggtg

SEQ ID NO: 199 - DNA, 19 bp, Artificial Sequence
  siRNA target
aagctgatgtgcaccgaca

SEQ ID NO: 200 - DNA, 19 bp, Artificial Sequence
  siRNA target
gatcaatggctacacagga

SEQ ID NO: 201 - DNA, 19 bp, Artificial Sequence
  siRNA target
tgtgtgacaaggtgcagaa

SEQ ID NO: 202 - DNA, 19 bp, Artificial Sequence
  siRNA target
ttccagtacctgccagata

SEQ ID NO: 203 - DNA, 19 bp, Artificial Sequence
  siRNA target
gaggagaaacgtaaaagga

SEQ ID NO: 204 - DNA, 19 bp, Artificial Sequence
  siRNA target
tatgagaccttcaagagca

SEQ ID NO: 205 - DNA, 19 bp, Artificial Sequence
  siRNA target
atgagaccttcaagagcat

SEQ ID NO: 206 - DNA, 19 bp, Artificial Sequence
  siRNA target
aagagcatcatgaagaaga

SEQ ID NO: 207 - DNA, 19 bp, Artificial Sequence
  siRNA target
tggcaacagcacagaccca

SEQ ID NO: 208 - DNA, 19 bp, Artificial Sequence
  siRNA target
caagatcaatggctacaca

SEQ ID NO: 209 - DNA, 19 bp, Artificial Sequence
  siRNA target
ggaagagccaactgtgtga

SEQ ID NO: 210 - DNA, 19 bp, Artificial Sequence
  siRNA target
ccaaacaggaggtgatcga

SEQ ID NO: 211 - DNA, 19 bp, Artificial Sequence
  siRNA target
ggacaaggcctctgtgaaa

SEQ ID NO: 212 - DNA, 19 bp, Artificial Sequence
  siRNA target
ggaaactggtggagagact

SEQ ID NO: 213 - DNA, 19 bp, Artificial Sequence
  siRNA target
gggagaagctggccgagaa

SEQ ID NO: 214 - DNA, 19 bp, Artificial Sequence
  siRNA target
gcgaggagcttctgcattt

SEQ ID NO: 215 - DNA, 19 bp, Artificial Sequence
  siRNA target
ggatcgaggacatgaggaa

SEQ ID NO: 216 - DNA, 19 bp, Artificial Sequence
  siRNA target
cggccaggatcgaggacat

SEQ ID NO: 217 - DNA, 19 bp, Artificial Sequence
  siRNA target
gaggaatgcagctggaaga

SEQ ID NO: 218 - DNA, 19 bp, Artificial Sequence
  siRNA target
tcgataagctgaaggagga

SEQ ID NO: 219 - DNA, 19 bp, Artificial Sequence
  siRNA target
tggagaagctcgatctgaa

SEQ ID NO: 220 - DNA, 19 bp, Artificial Sequence
  siRNA target
aaacaggaggtgatcgata

SEQ ID NO: 221 - DNA, 19 bp, Artificial Sequence
  siRNA target
ccaaacaggaggtgatcga

SEQ ID NO: 222 - DNA, 19 bp, Artificial Sequence
  siRNA target
agcagatggctgaggacaa

SEQ ID NO: 223 - DNA, 19 bp, Artificial Sequence
  siRNA target
agaagctggccgagaagaa

SEQ ID NO: 224 - DNA, 19 bp, Artificial Sequence
  siRNA target
gcttggaggctgccactaa

SEQ ID NO: 225 - DNA, 19 bp, Artificial Sequence
  siRNA target
tggccaaacaggaggtgat

SEQ ID NO: 226 - DNA, 19 bp, Artificial Sequence
  siRNA target
ggaagagccaactgtgtga

SEQ ID NO: 227 - DNA, 19 bp, Artificial Sequence
  siRNA target
aacaggaggtgatcgataa

SEQ ID NO: 228 - DNA, 19 bp, Artificial Sequence
  siRNA target
gaatgcagctggaagatct

SEQ ID NO: 229 - DNA, 19 bp, Artificial Sequence
  siRNA target
agagggaggagaaggagtt

SEQ ID NO: 230 - DNA, 19 bp, Artificial Sequence
  siRNA target
aggcggacttccaggctga

SEQ ID NO: 231 - DNA, 19 bp, Artificial Sequence
  siRNA target
tggagcagctgcagaggga

SEQ ID NO: 232 - DNA, 19 bp, Artificial Sequence
  siRNA target
gagcagctgcagagggagt

SEQ ID NO: 233 - DNA, 19 bp, Artificial Sequence
  siRNA target
cgtactgggcgaagagtct

SEQ ID NO: 234 - DNA, 19 bp, Artificial Sequence
  siRNA target
ggaggagaatcaagagctc

SEQ ID NO: 235 - DNA, 19 bp, Artificial Sequence
  siRNA target
ccagccagagggaggagaa

SEQ ID NO: 236 - DNA, 19 bp, Artificial Sequence
  siRNA target
gccagagggaggagaagga

SEQ ID NO: 237 - DNA, 19 bp, Artificial Sequence
  siRNA target
tgaagaggcagaaggagca

SEQ ID NO: 238 - DNA, 19 bp, Artificial Sequence
  siRNA target
tggaagatctcaaacagca

SEQ ID NO: 239 - DNA, 19 bp, Artificial Sequence
  siRNA target
ctgaagaggcagaaggagc

SEQ ID NO: 240 - DNA, 19 bp, Artificial Sequence
  siRNA target
aagaatacgacaaccacat

SEQ ID NO: 241 - DNA, 19 bp, Artificial Sequence
  siRNA target
gcagagggagtacagcaaa

SEQ ID NO: 242 - DNA, 19 bp, Artificial Sequence
  siRNA target
cagcagatcaggacgtact

SEQ ID NO: 243 - DNA, 19 bp, Artificial Sequence
  siRNA target
agtgagcggaagcgaggaa

SEQ ID NO: 244 - DNA, 19 bp, Artificial Sequence
  siRNA target
gagccaactgtgtgagatg

SEQ ID NO: 245 - DNA, 19 bp, Artificial Sequence
  siRNA target
ctgaaggcccaggcggata

SEQ ID NO: 246 - DNA, 19 bp, Artificial Sequence
  siRNA target
cagagggagtacagcaaac

SEQ ID NO: 247 - DNA, 19 bp, Artificial Sequence
  siRNA target
caggaaactggtggagaga

SEQ ID NO: 248 - DNA, 19 bp, Artificial Sequence
  siRNA target
tgaataggcacctctggaa

SEQ ID NO: 249 - DNA, 19 bp, Artificial Sequence
  siRNA target
gaggctgccactaaggaat

SEQ ID NO: 250 - DNA, 19 bp, Artificial Sequence
  siRNA target
cccagttgcaggtggccta

SEQ ID NO: 251 - DNA, 19 bp, Artificial Sequence
  siRNA target
gggagtacagcaaactgaa

SEQ ID NO: 252 - DNA, 19 bp, Artificial Sequence
  siRNA target
agcgctgcctggaggagaa

SEQ ID NO: 253 - DNA, 19 bp, Artificial Sequence
  siRNA target
aatcaagagctccgagatg

SEQ ID NO: 254 - DNA, 19 bp, Artificial Sequence
  siRNA target
cggcagagcaaccagattc

SEQ ID NO: 255 - DNA, 19 bp, Artificial Sequence
  siRNA target
agagatgccagcagcagat

SEQ ID NO: 256 - DNA, 19 bp, Artificial Sequence
  siRNA target
aggcccaggcggatatcta

SEQ ID NO: 257 - DNA, 19 bp, Artificial Sequence
  siRNA target
ctgcagagggagtacagca

SEQ ID NO: 258 - DNA, 19 bp, Artificial Sequence
  siRNA target
tgcagagggagtacagcaa

SEQ ID NO: 259 - DNA, 19 bp, Artificial Sequence
  siRNA target
tggacaccctgcagataca

SEQ ID NO: 260 - DNA, 19 bp, Artificial Sequence
  siRNA target
gcacctgccttcagaacag

SEQ ID NO: 261 - DNA, 21 bp, Artificial Sequence
  siRNA target
aagtccaatacctcactcgct

SEQ ID NO: 262 - DNA, 21 bp, Artificial Sequence
  siRNA target
aagcacgtcaaaagctacaga

SEQ ID NO: 263 - DNA, 19 bp, Artificial Sequence
  siRNA target
aatttctgtctcatcttaa

SEQ ID NO: 264 - DNA, 19 bp, Artificial Sequence
  siRNA target
ggtcttcaccaagtatcaa

SEQ ID NO: 265 - DNA, 19 bp, Artificial Sequence
  siRNA target
ggccatactcttacataat

SEQ ID NO: 266 - DNA, 19 bp, Artificial Sequence
  siRNA target
ggcaaggaaaataaaagat

SEQ ID NO: 267 - DNA, 19 bp, Artificial Sequence
  siRNA target
gcacgtcaaaagctacaga

SEQ ID NO: 268 - DNA, 19 bp, Artificial Sequence
  siRNA target
ggcactgtagtgaattatc

SEQ ID NO: 269 - DNA, 19 bp, Artificial Sequence
  siRNA target
gctagagttacctagctta

SEQ ID NO: 270 - DNA, 19 bp, Artificial Sequence
  siRNA target
aagaagagcctcaaccatt

SEQ ID NO: 271 - DNA, 19 bp, Artificial Sequence
  siRNA target
gtcaaaagctacagaatct

SEQ ID NO: 272 - DNA, 19 bp, Artificial Sequence
  siRNA target
cctcactcgctcagctata

SEQ ID NO: 273 - DNA, 19 bp, Artificial Sequence
  siRNA target
tcgctcagctataagaaga

SEQ ID NO: 274 - DNA, 19 bp, Artificial Sequence
  siRNA target
tgaaatgcctcaacaagca

SEQ ID NO: 275 - DNA, 19 bp, Artificial Sequence
  siRNA target
agctataagaagagcctca

SEQ ID NO: 276 - DNA, 19 bp, Artificial Sequence
  siRNA target
taatatgtctcttgctgat

SEQ ID NO: 277 - DNA, 19 bp, Artificial Sequence
  siRNA target
cctcaacaagcacgtcaaa

SEQ ID NO: 278 - DNA, 19 bp, Artificial Sequence
  siRNA target
ggagaaagtccaatacctc

SEQ ID NO: 279 - DNA, 19 bp, Artificial Sequence
  siRNA target
acaagcacgtcaaaagcta

SEQ ID NO: 280 - DNA, 19 bp, Artificial Sequence
  siRNA target
cgtcaaaagctacagaatc

SEQ ID NO: 281 - DNA, 19 bp, Artificial Sequence
  siRNA target
acgtcaaaagctacagaat

SEQ ID NO: 282 - DNA, 19 bp, Artificial Sequence
  siRNA target
gctacagaatctatttatc

SEQ ID NO: 283 - DNA, 19 bp, Artificial Sequence
  siRNA target
gctataagaagagcctcaa

SEQ ID NO: 284 - DNA, 19 bp, Artificial Sequence
  siRNA target
ataagaagagcctcaacca

SEQ ID NO: 285 - DNA, 19 bp, Artificial Sequence
  siRNA target
taagaagagcctcaaccat

SEQ ID NO: 286 - DNA, 19 bp, Artificial Sequence
  siRNAt target
gcctcaacaagcacgtcaa

SEQ ID NO: 287 - DNA, 19 bp, Artificial Sequence
  siRNA target
agaagagcctcaaccattg

SEQ ID NO: 288 - DNA, 19 bp, Artificial Sequence
  siRNA target
caatttctgtctcatctta

SEQ ID NO: 289 - DNA, 19 bp, Artificial Sequence
  siRNA target
tcaacaagcacgtcaaaag

SEQ ID NO: 290 - DNA, 19 bp, Artificial Sequence
  siRNA target
ttaatatgtctcttgctga

SEQ ID NO: 291 - DNA, 19 bp, Artificial Sequence
  siRNA target
gtctcttgctgatctgtat

SEQ ID NO: 292 - DNA, 19 bp, Artificial Sequence
  siRNA target
cttaatatgtctcttgctg

SEQ ID NO: 293 - DNA, 19 bp, Artificial Sequence
  siRNA target
tcagctataagaagagcct

SEQ ID NO: 294 - DNA, 19 bp, Artificial Sequence
  siRNA target
ccaatacctcactcgctca

SEQ ID NO: 295 - DNA, 19 bp, Artificial Sequence
  siRNA target
caaaagctacagaatctat

SEQ ID NO: 296 - DNA, 19 bp, Artificial Sequence
  siRNA target
catcttaatatgtctcttg

SEQ ID NO: 297 - DNA, 19 bp, Artificial Sequence
  siRNA target
agaaagtccaatacctcac

SEQ ID NO: 298 - DNA, 19 bp, Artificial Sequence
  siRNA target
ctataagaagagcctcaac

SEQ ID NO: 299 - DNA, 19 bp, Artificial Sequence
  siRNA target
actcgctcagctataagaa

SEQ ID NO: 300 - DNA, 19 bp, Artificial Sequence
  siRNA target
tcaaaagctacagaatcta

SEQ ID NO: 301 - DNA, 19 bp, Artificial Sequence
  siRNA target
tatcaatttctgtctcatc

SEQ ID NO: 302 - DNA, 19 bp, Artificial Sequence
  siRNA target
tgtctcttgctgatctgta

SEQ ID NO: 303 - DNA, 19 bp, Artificial Sequence
  siRNA target
aagcacgtcaaaagctaca

SEQ ID NO: 304 - DNA, 19 bp, Artificial Sequence
  siRNA target
gcacgtcaaaagctacaga

SEQ ID NO: 305 - DNA, 19 bp, Artificial Sequence
  siRNA target
tcaatttctgtctcatctt

SEQ ID NO: 306 - DNA, 19 bp, Artificial Sequence
  siRNA target
tgcctcaacaagcacgtca

SEQ ID NO: 307 - DNA, 19 bp, Artificial Sequence
  siRNA target
tccaatacctcactcgctc

SEQ ID NO: 308 - DNA, 19 bp, Artificial Sequence
  siRNA target
ctcactcgctcagctataa

SEQ ID NO: 309 - DNA, 19 bp, Artificial Sequence
  siRNA target
tacctcactcgctcagcta

SEQ ID NO: 310 - DNA, 19 bp, Artificial Sequence
  siRNA target
ctgtctcatcttaatatgt

SEQ ID NO: 311 - DNA, 19 bp, Artificial Sequence
  siRNA target
ctcaacaagcacgtcaaaa

SEQ ID NO: 312 - DNA, 19 bp, Artificial Sequence
  siRNA target
caacaagcacgtcaaaagc

SEQ ID NO: 313 - DNA, 19 bp, Artificial Sequence
  siRNA target
tataagaagagcctcaacc

SEQ ID NO: 314 - DNA, 19 bp, Artificial Sequence
  siRNA target
atatgtctcttgctgatct

SEQ ID NO: 315 - DNA, 19 bp, Artificial Sequence
  siRNA target
cgctcagctataagaagag

SEQ ID NO: 316 - DNA, 19 bp, Artificial Sequence
  siRNA target
atcaatttctgtctcatct

SEQ ID NO: 317 - DNA, 19 bp, Artificial Sequence
  siRNA target
tgtctcatcttaatatgtc

SEQ ID NO: 318 - DNA, 19 bp, Artificial Sequence
  siRNA target
gtctcatcttaatatgtct

SEQ ID NO: 319 - DNA, 19 bp, Artificial Sequence
  siRNA target
gtccaatacctcactcgct

SEQ ID NO: 320 - DNA, 19 bp, Artificial Sequence
  siRNA target
tgatgtaagttctgagtgt

SEQ ID NO: 321 - DNA, 19 bp, Artificial Sequence
  siRNA target
ccgagaaggtagacaattg

SEQ ID NO: 322 - DNA, 19 bp, Artificial Sequence
  siRNA target
tgaccgagaaggtagacaa

SEQ ID NO: 323 - DNA, 19 bp, Artificial Sequence
  siRNA target
aaagcaaccttctgatgta

SEQ ID NO: 324 - DNA, 19 bp, Artificial Sequence
  siRNA target
atgtaagttctgagtgtga

SEQ ID NO: 325 - DNA, 19 bp, Artificial Sequence
  siRNA target
gttctgagtgtgaccgaga

SEQ ID NO: 326 - DNA, 19 bp, Artificial Sequence
  siRNA target
gaccgagaaggtagacaat

SEQ ID NO: 327 - DNA, 19 bp, Artificial Sequence
  siRNA target
gcacccatgagttgtgaca

SEQ ID NO: 328 - DNA, 19 bp, Artificial Sequence
  siRNA target
ctacctccctacagacaga

SEQ ID NO: 329 - DNA, 19 bp, Artificial Sequence
  siRNA target
ccctacagacagagccaca

SEQ ID NO: 330 - DNA, 19 bp, Artificial Sequence
  siRNA target
gtgtgaccgagaaggtaga

SEQ ID NO: 331 - DNA, 19 bp, Artificial Sequence
  siRNA target
aagcaaccttctgatgtaa

SEQ ID NO: 332 - DNA, 19 bp, Artificial Sequence
  siRNA target
gagtgtgaccgagaaggta

SEQ ID NO: 333 - DNA, 19 bp, Artificial Sequence
  siRNA target
gcaaccttctgatgtaagt

SEQ ID NO: 334 - DNA, 19 bp, Artificial Sequence
  siRNA target
tgacaaatcaacacaaacc

SEQ ID NO: 335 - DNA, 19 bp, Artificial Sequence
  siRNA target
gcaaagcaaccttctgatg

SEQ ID NO: 336 - DNA, 19 bp, Artificial Sequence
  siRNA target
tgagtgtgaccgagaaggt

SEQ ID NO: 337 - DNA, 19 bp, Artificial Sequence
  siRNA target
caaagcaaccttctgatgt

SEQ ID NO: 338 - DNA, 19 bp, Artificial Sequence
  siRNA target
gcccagcacccatgagttg

SEQ ID NO: 339 - DNA, 19 bp, Artificial Sequence
  siRNA target
gtgacaaatcaacacaaac

SEQ ID NO: 340 - DNA, 19 bp, Artificial Sequence
  siRNA target
cctacagacagagccacaa

SEQ ID NO: 341 - DNA, 19 bp, Artificial Sequence
  siRNA target
agtgtgaccgagaaggtag

SEQ ID NO: 342 - DNA, 19 bp, Artificial Sequence
  siRNA target
ggcaaagcaaccttctgat

SEQ ID NO: 343 - DNA, 19 bp, Artificial Sequence
  siRNA target
ctccctacagacagagcca

SEQ ID NO: 344 - DNA, 19 bp, Artificial Sequence
  siRNA target
ctacagacagagccacaag

SEQ ID NO: 345 - DNA, 19 bp, Artificial Sequence
  siRNA target
acacaaaccccaagtcctc

SEQ ID NO: 346 - DNA, 19 bp, Artificial Sequence
  siRNA target
gccaggccttcaaccacta

SEQ ID NO: 347 - DNA, 19 bp, Artificial Sequence
  siRNA target
gagaaggtagacaattgca

SEQ ID NO: 348 - DNA, 19 bp, Artificial Sequence
  siRNA target
aaatcaacacaaaccccaa

SEQ ID NO: 349 - DNA, 19 bp, Artificial Sequence
  siRNA target
ggtagacaattgcagcctg

SEQ ID NO: 350 - DNA, 19 bp, Artificial Sequence
  siRNA target
ccctacctccctacagaca

SEQ ID NO: 351 - DNA, 19 bp, Artificial Sequence
  siRNA target
gagcccagcacccatgagt

SEQ ID NO: 352 - DNA, 19 bp, Artificial Sequence
  siRNA target
cttgccaggccttcaacca

SEQ ID NO: 353 - DNA, 19 bp, Artificial Sequence
  siRNA target
agaaggtagacaattgcag

SEQ ID NO: 354 - DNA, 19 bp, Artificial Sequence
  siRNA target
gaaggtagacaattgcagc

SEQ ID NO: 355 - DNA, 19 bp, Artificial Sequence
  siRNA target
tctgagtgtgaccgagaag

SEQ ID NO: 356 - DNA, 19 bp, Artificial Sequence
  siRNA target
gtgaccgagaaggtagaca

SEQ ID NO: 357 - DNA, 19 bp, Artificial Sequence
  siRNA target
aagttctgagtgtgaccga

SEQ ID NO: 358 - DNA, 19 bp, Artificial Sequence
  siRNA target
tgccaggccttcaaccact

SEQ ID NO: 359 - DNA, 19 bp, Artificial Sequence
  siRNA target
cgagaaggtagacaattgc

SEQ ID NO: 360 - DNA, 19 bp, Artificial Sequence
  siRNA target
gtaagttctgagtgtgacc

SEQ ID NO: 361 - DNA, 19 bp, Artificial Sequence
  siRNA target
tctgatgtaagttctgagt

SEQ ID NO: 362 - DNA, 19 bp, Artificial Sequence
  siRNA target
gcccctacctccctacaga

SEQ ID NO: 363 - DNA, 19 bp, Artificial Sequence
  siRNA target
acaggagcccagcacccat

SEQ ID NO: 364 - DNA, 19 bp, Artificial Sequence
  siRNA target
accgagaaggtagacaatt

SEQ ID NO: 365 - DNA, 19 bp, Artificial Sequence
  siRNA target
agcccagcacccatgagtt

SEQ ID NO: 366 - DNA, 19 bp, Artificial Sequence
  siRNA target
caaatcaacacaaacccca

SEQ ID NO: 367 - DNA, 19 bp, Artificial Sequence
  siRNA target
cctccttgccaggccttca

SEQ ID NO: 368 - DNA, 19 bp, Artificial Sequence
  siRNA target
cttctgatgtaagttctga

SEQ ID NO: 369 - DNA, 19 bp, Artificial Sequence
  siRNA target
aggtagacaattgcagcct

SEQ ID NO: 370 - RNA, 19 nt, Artificial Sequence
  siRNA strand
aguggaugagggagcaggc

SEQ ID NO: 371 - RNA, 19 nt, Artificial Sequence
  siRNA strand
gccugcucccucauccacu

SEQ ID NO: 372 - RNA, 19 nt, Artificial Sequence
  siRNA strand
gcacacaguggaugaggga

SEQ ID NO: 373 - RNA, 19 nt, Artificial Sequence
  siRNA strand
ucccucauccacugugugc

SEQ ID NO: 374 - RNA, 19 nt, Artificial Sequence
  siRNA strand
ugaagugcacacaguggau

SEQ ID NO: 375 - RNA, 19 nt, Artificial Sequence
  siRNA strand
auccacugugugcacuuca

SEQ ID NO: 376 - RNA, 19 nt, Artificial Sequence
  siRNA strand
cacacaguggaugagggag

SEQ ID NO: 377 - RNA, 19 nt, Artificial Sequence
  siRNA strand
cucccucauccacugugug

SEQ ID NO: 378 - RNA, 19 nt, Artificial Sequence
  siRNA strand
gcacacaguagaugaggga

SEQ ID NO: 379 - RNA, 19 nt, Artificial Sequence
  siRNA strand
ucccucaucuacugugugc

SEQ ID NO: 380 - RNA, 19 nt, Artificial Sequence
  siRNA strand
ugaagugcacacaguagau

SEQ ID NO: 381 - RNA, 19 nt, Artificial Sequence
  siRNA strand
aucuacugugugcacuuca

SEQ ID NO: 382 - RNA, 19 nt, Artificial Sequence
  siRNA strand
aguagaugagggagcaggc

SEQ ID NO: 383 - RNA, 19 nt, Artificial Sequence
  siRNA strand
gccugcucccucaucuacu

SEQ ID NO: 384 - RNA, 19 nt, Artificial Sequence
  siRNA strand
cacacaguagaugagggag

SEQ ID NO: 385 - RNA, 19 nt, Artificial Sequence
  siRNA strand
cucccucaucuacugugug

SEQ ID NO: 386 - RNA, 19 nt, Artificial Sequence
  siRNA strand
ggcgcagacuuccaaaggc

SEQ ID NO: 387 - RNA, 19 nt, Artificial Sequence
  siRNA strand
gccuuuggaagucugcgcc

SEQ ID NO: 388 - RNA, 19 nt, Artificial Sequence
  siRNA strand
cacaagggcgcagacuucc

SEQ ID NO: 389 - RNA, 19 nt, Artificial Sequence
  siRNA strand
ggaagucugcgcccuugug

SEQ ID NO: 390 - RNA, 19 nt, Artificial Sequence
  siRNA strand
gcagggcacaagggcgcag

SEQ ID NO: 391 - RNA, 19 nt, Artificial Sequence
  siRNA strand
cugcgcccuugugcccugc

SEQ ID NO: 392 - RNA, 19 nt, Artificial Sequence
  siRNA strand
acaagggcgcagacuucca

SEQ ID NO: 393 - RNA, 19 nt, Artificial Sequence
  siRNA strand
uggaagucugcgcccuugu

SEQ ID NO: 394 - RNA, 19 nt, Artificial Sequence
  siRNA strand
cacaagggcacagacuucc

SEQ ID NO: 395 - RNA, 19 nt, Artificial Sequence
  siRNA strand
ggaagucugugcccuugug

SEQ ID NO: 396 - RNA, 19 nt, Artificial Sequence
  siRNA strand
gcagggcacaagggcacag

SEQ ID NO: 397 - RNA, 19 nt, Artificial Sequence
  siRNA strand
cugugcccuugugcccugc

SEQ ID NO: 398 - RNA, 19 nt, Artificial Sequence
  siRNA strand
ggcacagacuuccaaaggc

SEQ ID NO: 399 - RNA, 19 nt, Artificial Sequence
  siRNA strand
gccuuuggaagucugugcc

SEQ ID NO: 400 - RNA, 19 nt, Artificial Sequence
  siRNA strand
acaagggcacagacuucca

SEQ ID NO: 401 - RNA, 19 nt, Artificial Sequence
  siRNA strand
uggaagucugugcccuugu

SEQ ID NO: 402 - RNA, 19 nt, Artificial Sequence
  siRNA strand
caaugguacagcucuuccu

SEQ ID NO: 403 - RNA, 19 nt, Artificial Sequence
  siRNA strand
aggaagagcuguaccauug

SEQ ID NO: 404 - RNA, 19 nt, Artificial Sequence
  siRNA strand
caucccaaugguacagcuc

SEQ ID NO: 405 - RNA, 19 nt, Artificial Sequence
  siRNA strand
gagcuguaccauugggaug

SEQ ID NO: 406 - RNA, 19 nt, Artificial Sequence
  siRNA strand
ccaucccaaugguacagcu

SEQ ID NO: 407 - RNA, 19 nt, Artificial Sequence
  siRNA strand
agcuguaccauugggaugg

SEQ ID NO: 408 - RNA, 19 nt, Artificial Sequence
  siRNA strand
uuguggccaucccaauggu

SEQ ID NO: 409 - RNA, 19 nt, Artificial Sequence
  siRNA strand
accauugggauggccacaa

SEQ ID NO: 410 - RNA, 19 nt, Artificial Sequence
  siRNA strand
caacgguacagcucuuccu

SEQ ID NO: 411 - RNA, 19 nt, Artificial Sequence
  siRNA strand
aggaagagcuguaccguug

SEQ ID NO: 412 - RNA, 19 nt, Artificial Sequence
  siRNA strand
caucccaacgguacagcuc

SEQ ID NO: 413 - RNA, 19 nt, Artificial Sequence
  siRNA strand
gagcuguaccguugggaug

SEQ ID NO: 414 - RNA, 19 nt, Artificial Sequence
  siRNA strand
ccaucccaacgguacagcu

SEQ ID NO: 415 - RNA, 19 nt, Artificial Sequence
  siRNA strand
agcuguaccguugggaugg

SEQ ID NO: 416 - RNA, 19 nt, Artificial Sequence
  siRNA strand
uuguggccaucccaacggu

SEQ ID NO: 417 - RNA, 19 nt, Artificial Sequence
  siRNA stand
accguugggauggccacaa

SEQ ID NO: 418 - RNA, 19 nt, Artificial Sequence
  siRNA strand
gucggcaagcagagcuccc

SEQ ID NO: 419 - RNA, 19 nt, Artificial Sequence
  siRNA strand
gggagcucugcuugccgac

SEQ ID NO: 420 - RNA, 19 nt, Artificial Sequence
  siRNA strand
agccagucggcaagcagag

SEQ ID NO: 421 - RNA, 19 nt, Artificial Sequence
  siRNA strand
cucugcuugccgacuggcu

SEQ ID NO: 422 - RNA, 21 nt, Artificial Sequence
  siRNA strand
cagccagucggcaagcagagc

SEQ ID NO: 423 - RNA, 21 nt, Artificial Sequence
  siRNA strand
gcucugcuugccgacuggcug

SEQ ID NO: 424 - RNA, 19 nt, Artificial Sequence
  siRNA strand
gucucacagccagucggca

SEQ ID NO: 425 - RNA, 19 nt, Artificial Sequence
  siRNA strand
ugccgacuggcugugagac

SEQ ID NO: 426 - RNA, 19 nt, Artificial Sequence
  siRNA strand
gucagcaagcagagcuccc

SEQ ID NO: 427 - RNA, 19 nt, Artificial Sequence
  siRNA strand
gggagcucugcuugcugac

SEQ ID NO: 428 - RNA, 19 nt, Artificial Sequence
  siRNA strand
agccagucagcaagcagag

SEQ ID NO: 429 - RNA, 19 nt, Artificial Sequence
  siRNA strand
cucugcuugcugacuggcu

SEQ ID NO: 430 - RNA, 21 nt, Artificial Sequence
  siRNA strand
cagccagucagcaagcagagc

SEQ ID NO: 431 - RNA, 21 nt, Artificial Sequence
  siRNA strand
gcucugcuugcugacuggcug

SEQ ID NO: 432 - RNA, 19 nt, Artificial Sequence
  siRNA strand
gucucacagccagucagca

SEQ ID NO: 433 - RNA, 19 nt, Artificial Sequence
  siRNA strand
ugcugacuggcugugagac

SEQ ID NO: 434 - RNA, 19 nt, Artificial Sequence
  siRNA strand
cacauacauuagcucaaac

SEQ ID NO: 435 - RNA, 19 nt, Artificial Sequence
  siRNA strand
guuugagcuaauguaugug

SEQ ID NO: 436 - RNA, 19 nt, Artificial Sequence
  siRNA strand
cagcgucacauacauuagc

SEQ ID NO: 437 - RNA, 19 nt, Artificial Sequence
  siRNA strand
gcuaauguaugugacgcug

SEQ ID NO: 438 - RNA, 19 nt, Artificial Sequence
  siRNA strand
acauacauuagcucaaacu

SEQ ID NO: 439 - RNA, 19 nt, Artificial Sequence
  siRNA strand
aguuugagcuaauguaugu

SEQ ID NO: 440 - RNA, 19 nt, Artificial Sequence
  siRNA strand
cauuagcucaaacugguug

SEQ ID NO: 441 - RNA, 19 nt, Artificial Sequence
  siRNA strand
caaccaguuugagcuaaug

SEQ ID NO: 442 - RNA, 19 nt, Artificial Sequence
  siRNA strand
cacauacaucagcucaaac

SEQ ID NO: 443 - RNA, 19 nt, Artificial Sequence
  siRNA strand
guuugagcugauguaugug

SEQ ID NO: 444 - RNA, 19 nt, Artificial Sequence
  siRNA strand
cagcgucacauacaucagc

SEQ ID NO: 445 - RNA, 19 nt, Artificial Sequence
  siRNA strand
gcugauguaugugacgcug

SEQ ID NO: 446 - RNA, 19 nt, Artificial Sequence
  siRNA strand
acauacaucagcucaaacu

SEQ ID NO: 447 - RNA, 19 nt, Artificial Sequence
  siRNA strand
aguuugagcugauguaugu

SEQ ID NO: 448 - RNA, 19 nt, Artificial Sequence
  siRNA strand
caucagcucaaacugguug

SEQ ID NO: 449 - RNA, 19 nt, Artificial Sequence
  siRNA strand
caaccaguuugagcugaug

SEQ ID NO: 450 - RNA, 19 nt, Artificial Sequence
  siRNA strand
caacaucaaagcaucuuga

SEQ ID NO: 451 - RNA, 19 nt, Artificial Sequence
  siRNA strand
ucaagaugcuuugauguug

SEQ ID NO: 452 - RNA, 19 nt, Artificial Sequence
  siRNA strand
ccggccaacaucaaagcau

SEQ ID NO: 453 - RNA, 19 nt, Artificial Sequence
  siRNA strand
augcuuugauguuggccgg

SEQ ID NO: 454 - RNA, 19 nt, Artificial Sequence
  siRNA strand
uccggccaacaucaaagca

SEQ ID NO: 455 - RNA, 19 nt, Artificial Sequence
  siRNA strand
ugcuuugauguuggccgga

SEQ ID NO: 456 - RNA, 19 nt, Artificial Sequence
  siRNA strand
caaguuuccggccaacauc

SEQ ID NO: 457 - RNA, 19 nt, Artificial Sequence
  siRNA strand
gauguuggccggaaacuug

SEQ ID NO: 458 - RNA, 19 nt, Artificial Sequence
  siRNA strand
caaaaucaaagcaucuuga

SEQ ID NO: 459 - RNA, 19 nt, Artificial Sequence
  siRNA strand
ucaagaugcuuugauuuug

SEQ ID NO: 460 - RNA, 19 nt, Artificial Sequence
  siRNA strand
ccggccaaaaucaaagcau

SEQ ID NO: 461 - RNA, 19 nt, Artificial Sequence
  siRNA strand
augcuuugauuuuggccgg

SEQ ID NO: 462 - RNA, 19 nt, Artificial Sequence
  siRNA strand
uccggccaaaaucaaagca

SEQ ID NO: 463 - RNA, 19 nt, Artificial Sequence
  siRNA strand
ugcuuugauuuuggccgga

SEQ ID NO: 464 - RNA, 19 nt, Artificial Sequence
  siRNA strand
caaguuuccggccaaaauc

SEQ ID NO: 465 - RNA, 19 nt, Artificial Sequence
  siRNA strand
gauuuuggccggaaacuug

SEQ ID NO: 466 - RNA, 19 nt, Artificial Sequence
  siRNA strand
gaauuacuguccccaucuc

SEQ ID NO: 467 - RNA, 19 nt, Artificial Sequence
  siRNA strand
gagauggggacaguaauuc

SEQ ID NO: 468 - RNA, 19 nt, Artificial Sequence
  siRNA strand
gcguugaauuacugucccc

SEQ ID NO: 469 - RNA, 19 nt, Artificial Sequence
  siRNA strand
ggggacaguaauucaacgc

SEQ ID NO: 470 - RNA, 19 nt, Artificial Sequence
  siRNA strand
agcguugaauuacuguccc

SEQ ID NO: 471 - RNA, 19 nt, Artificial Sequence
  siRNA strand
gggacaguaauucaacgcu

SEQ ID NO: 472 - RNA, 19 nt, Artificial Sequence
  siRNA strand
ucuucuagcguugaauuac

SEQ ID NO: 473 - RNA, 19 nt, Artificial Sequence
  siRNA strand
guaauucaacgcuagaaga

SEQ ID NO: 474 - RNA, 19 nt, Artificial Sequence
  siRNA strand
gaaguacuguccccaucuc

SEQ ID NO: 475 - RNA, 19 nt, Artificial Sequence
  siRNA strand
gagauggggacaguacuuc

SEQ ID NO: 476 - RNA, 19 nt, Artificial Sequence
  siRNA strand
gcguugaaguacugucccc

SEQ ID NO: 477 - RNA, 19 nt, Artificial Sequence
  siRNA strand
ggggacaguacuucaacgc

SEQ ID NO: 478 - RNA, 19 nt, Artificial Sequence
  siRNA strand
agcguugaaguacuguccc

SEQ ID NO: 479 - RNA, 19 nt, Artificial Sequence
  siRNA strand
gggacaguacuucaacgcu

SEQ ID NO: 480 - RNA, 19 nt, Artificial Sequence
  siRNA strand
ucuucuagcguugaaguac

SEQ ID NO: 481 - RNA, 19 nt, Artificial Sequence
  siRNA strand
guacuucaacgcuagaaga

SEQ ID NO: 482 - RNA, 19 nt, Artificial Sequence
  siRNA stand
uagcguugaauuacugucc

SEQ ID NO: 483 - RNA, 19 nt, Artificial Sequence
  siRNA strand
ggacaguaauucaacgcua

SEQ ID NO: 484 - RNA, 19 nt, Artificial Sequence
  siRNA strand
uagcguugacuuacugucc

SEQ ID NO: 485 - RNA, 19 nt, Artificial Sequence
  siRNA strand
ggacaguaagucaacgcua

SEQ ID NO: 486 - RNA, 19 nt, Artificial Sequence
  siRNA strand
uagcguugaaguacugucc

SEQ ID NO: 487 - RNA, 19 nt, Artificial Sequence
  siRNA strand
ggacaguacuucaacgcua

SEQ ID NO: 488 - RNA, 19 nt, Artificial Sequence
  siRNA strand
uagcguugauguacugucc

SEQ ID NO: 489 - RNA, 19 nt, Artificial Sequence
  siRNA strand
ggacaguacaucaacgcua

SEQ ID NO: 490 - RNA, 19 nt, Artificial Sequence
  siRNA strand
cacauacauuggcucaaac

SEQ ID NO: 491 - RNA, 19 nt, Artificial Sequence
  siRNA strand
guuugagccaauguaugug

SEQ ID NO: 492 - RNA, 19 nt, Artificial Sequence
  siRNA strand
cacauacaucggcucaaac

SEQ ID NO: 493 - RNA, 19 nt, Artificial Sequence
  siRNA strand
guuugagccgauguaugug

SEQ ID NO: 494 - DNA, 20 bp, Artificial Sequence
  siRNA target
tgatgtaagttctgagtgtg

SEQ ID NO: 495 - DNA, 10636 bp, Homo sapiens
gaggagagagcagagtataccgcagacatcatttctactacagtggcggagccgtacaggacctgtttcactgcagggggatccaaaacaagccccgtggagcagcagccagagcaacagcagccgcaagacattgtttctctccctctgcccccccttccccacgcaaccccagatccatttacactttacagttttacctcacaaaaactactacaagcaccaagctccctgatggaaaggagcatcgtgcatcaagtcaccagggtggtccattcaagctgcagatttgtttgtcatccttgtacagcaatctcctcctccactgccactacagggaagtgcatcacatgtcagcatactggagcatagtgaaagagtctattttgaagcttcaaacttagtgctgctgcagaccaggaacaagagagaaagagtggatttcagcctgcacggatggtcttgaaacacaaatggtttttggtctaggcgttttacactgagattctccactgccaccctttctactcaagcaaaatcttcgtgaaaagatctgctgcaaggaactgatagcttatggttctccattgtgatgaaagcacatggtacagttttccaaagaaattagaccattttcttcgtgagaaagaaatcgacgtgctgttttcatagggtatttctcacttctctgtgaaaggaagaaagaacacgcctgagcccaagagccctcaggagccctccagagcctgtgggaagtctccatggtgaagtataggctgaggctacctgtgaacagtacgcagtgaatgttcatccagagctgctgttggcggattgtacccacggggagatgattcctcatgaagagcctggatcccctacagaaatcaaatgtgactttccgtttatcagactaaaatcagagccatccagacagtgaaacagtcaccgtggaggggggacggcgaaaaatgaaatccaaccaagagcggagcaacgaatgcctgcctcccaagaagcgcgagatccccgccaccagccggtcctccgaggagaaggcccctaccctgcccagcgacaaccaccgggtggagggcacagcatggctcccgggcaaccctggtggccggggccacgggggcgggaggcatgggccggcagggacctcggtggagcttggtttacaacagggaataggtttacacaaagcattgtccacagggctggactactccccgcccagcgctcccaggtctgtccccgtggccaccacgctgcctgccgcgtacgccaccccgcagccagggaccccggtgtcccccgtgcagtacgctcacctgccgcacaccttccagttcattgggtcctcccaatacagtggaacctatgccagcttcatcccatcacagctgatccccccaaccgccaaccccgtcaccagtgcagtggcctcggccgcaggggccaccactccatcccagcgctcccagctggaggcctattccactctgctggccaacatgggcagtctgagccagacgccgggacacaaggctgagcagcagcagcagcagcagcagcagcagcagcagcagcatcagcatcagcagcagcagcagcagcagcagcagcagcagcagcagcagcacctcagcagggctccggggctcatcaccccggggtcccccccaccagcccagcagaaccagtacgtccacatttccagttctccgcagaacaccggccgcaccgcctctcctccggccatccccgtccacctccacccccaccagacgatgatcccacacacgctcaccctggggcccccctcccaggtcgtcatgcaatacgccgactccggcagccactttgtccctcgggaggccaccaagaaagctgagagcagccggctgcagcaggccatccaggccaaggaggtcctgaacggtgagatggagaagagccggcggtacggggccccgtcctcagccgacctgggcctgggcaaggcaggcggcaagtcggttcctcacccgtacgagtccaggcacgtggtggtccacccgagcccctcagactacagcagtcgtgatccttcgggggtccgggcctctgtgatggtcctgcccaacagcaacacgcccgcagctgacctggaggtgcaacaggccactcatcgtgaagcctccccttctaccctcaacgacaaaagtggcctgcatttagggaagcctggccaccggtcctacgcgctctcaccccacacggtcattcagaccacacacagtgcttcagagccactcccggtgggactgccagccacggccttctacgcagggactcaaccccctgtcatcggctacctgagcggccagcagcaagcaatcacctacgccggcagcctgccccagcacctggtgatccccggcacacagcccctgctcatcccggtcggcagcactgacatggaagcgtcgggggcagccccggccatagtcacgtcatccccccagtttgctgcagtgcctcacacgttcgtcaccaccgcccttcccaagagcgagaacttcaaccctgaggccctggtcacccaggccgcctacccagccatggtgcaggcccagatccacctgcctgtggtgcagtccgtggcctccccggcggcggctccccctacgctgcctccctacttcatgaaaggctccatcatccagttggccaacggggagctaaagaaggtggaagacttaaaaacagaagatttcatccagagtgcagagataagcaacgacctgaagatcgactccagcaccgtagagaggattgaagacagccatagcccgggcgtggccgtgatacagttcgccgtcggggagcaccgagcccaggtcagcgttgaagttttggtagagtatcctttttttgtgtttggacagggctggtcatcctgctgtccggagagaaccagccagctctttgatttgccgtgttccaaactctcagttggggatgtctgcatctcgcttaccctcaagaacctgaagaacggctctgttaaaaagggccagcccgtggatcccgccagcgtcctgctgaagcactcaaaggccgacggcctggcgggcagcagacacaggtatgccgagcaggaaaacggaatcaaccaggggagtgcccagatgctctctgagaatggcgaactgaagtttccagagaaaatgggattgcctgcagcgcccttcctcaccaaaatagaacccagcaagcccgcggcaacgaggaagaggaggtggtcggcgccagagagccgcaaactggagaagtcagaagacgaaccacctttgactcttcctaagccttctctaattcctcaggaggttaagatttgcattgaaggccggtctaatgtaggcaagtagaggcagcgtgggggaaaggaaacgtggctctcccttatcatttgtatccagattactgtactgtaggctaaaataacacagtatttacatgttatcttcttaattttaggtttctgttctaaccttgtcattagagttacagcaggtgtgtcgcaggagactggtgcatatgctttttccacgagtgtctgtcagtgagcgggcgggaggaagggcacagcaggagcggtcagggctccaggcatccccggggaagaaaggaacggggcttcacagtgcctgccttctctagcggcacagaagcagccgggggcgctgactcccgctagtgtcaggagaaaagtcccgtgggaagggtcctgcaggggtgcagggttgcacgcatgtgggggtgcacaggcgctgtggcggcgagtgagggtctctttttctctgcctccctctgcctcactctcttgctatcggcatgggccgggggggttcagagcagtgtcctcctggggttcccacgtgcaaaatcaacatcaggaacccagcttcagggcatcgcggagacgcgtcagatggcagatttggaaagttaaccatttaaaagaacatttttctctccaacatattttacaataaaagcaacttttaattgtatagatatatatttccccctatggggcctgactgcactgatatatattttttttaaagagcaactgccacatgcgggatttcatttctgctttttactagtgcagcgatgtcaccagggtgttgtggtggacagggaagcccctgctgtcatggccccacatggggtaaggggggttgggggtgggggagagggagagagcgaacacccacgctggtttctgtgcagtgttaggaaaaccaatcaggttattgcattgacttcactcccaagaggtagatgcaaactgcccttcagtgagagcaacagaagctcttcacgttgagtttgcgaaatctttttgtctttgaactctagtactgtttatagttcatgactatggacaactcgggtgccactttttttttttttcagattccagtgtgacatgaggaattagattttgaagatgagcatatattactatctttaagcatttaaaaatactgttcacactttattaccaagcatcttggtctctcattcaacaagtactgtatctcactttaaactctttggggaaaaaacaaaaacaaaaaaaactaagttgctttctttttttcaacactgtaactacatttcagctctgcagaattgctgaagagcaagatattgaaagtttcaatgtggtttaaagggatgaatgtgaattatgaactagtatgtgacaataaatgaccaccaagtactacctgacgggaggcacttttcactttgatgtctgagaatcagttcaaggcatatgcagagttggcagagaaactgagagaaaagggatggagaagagaatactcatttttgtccagtgtttttctttttaagatgaacttttaaagaaccttgcgatttgcacatattgagtttataacttgtgtgatattcctgcagtttttatccaataacattgtgggaaaggtttgggggactgaacgagcataaataaatgtagcaaaatttctttctaacctgcctaaactctaggccattttataaggttatgttcctttgaaaattcattttggtctttttaccacatctgtcacaaaaagccaggtcttagcgggctcttagaaactctgagaattttcttcagattcattgagagagttttccataaagacatttatatatgtgagcaagattttttttaaacaattactttattattgttgttattaatgttattttcagaatggcttttttttttctattcaaaatcaaatcgagatttaatgtttggtacaaacccagaaagggtatttcatagtttttaaacctttcattcccagagatccgaaatatcatttgtgggttttgaatgcatctttaaagtgctttaaaaaaaagttttataagtagggagaaatttttaaatattcttacttggatggctgcaactaaactgaacaaatacctgacttttcttttaccccattgaaaatagtactttcttcgtttcacaaattaaaaaaaaaatctggtatcaacccacattttggctgtctagtattcatttacatttagggttcaccaggactaatgatttttataaaccgttttctggggtgtaccaaaaacatttgaataggtttagaatagctagaatagttccttgactttcctcgaatttcattaccctctcagcatgcttgcagagagctgggtgggctcattcttgcagtcatactgcttatttagtgctgtattttttaaacgtttctgttcagagaacttgcttaatcttccatatattctgctcagggcacttgcaattattaggttttgtttttctttttgttttttagcctttgatggtaagaggaatacgggctgccacatagactttgttctcattaatatcactatttacaactcatgtggactcagaaaaacacacaccaccttttggcttacttcgagtattgaattgactggatccactaaaccaacactaagatgggaaaacacacatggtttggagcaataggaacatcatcataatttttgtggttctatttcaggtataggaattataaaataattggttctttctaaacacttgtcccatttcattctcttgcttttttagcatgtgcaatactttctgtgccaatagagtctgaccagtgtgctatatagttaaagctcattcccttttggctttttccttgtttggttgatcttccccattctggccagagcagggctggagggaaggagccaggagggagagagcctcccacctttcccctgctgcggatgctgagtgctggggcggggagccttcaggagccccgtgcgtctgccgccacgttgcagaaagagccagccaaggagacccgggggaggaaccgcagtgtcccctgtcaccacacggaatagtgaatgtggagtgtggagaggaaggaggcagattcatttctaagacgcactctggagccatgtagcctggagtcaacccattttccacggtcttttctgcaagtgggcaggcccctcctcggggtctgtgtccttgagacttggagccctgcctctgagcctggacgggaagtgtggcctgttgtgtgtgtgcgttctgagcgtgttggccagtggctgtggaggggaccacctgccacccacggtcaccactcccttgtggcagctttctcttcaaataggaagaacgcacagagggcaggagcctcctgtttgcagacgttggcgggccccgaggctcccagagcagcctctgtcaccgcttctgtgtagcaaacattaacgatgacaggggtagaaattcttcggtgccgttcagcttacaaggatcagccatgtgcctctgtactatgtccactttgcaatatttaccgacagccgtcttttgttctttctttcctgttttccatttttaaactagtaacagcaggccttttgcgtttacaatggaacacaatcaccaagaaattagtcagggcgaaaagaaaaaaataatactattaataagaaaccaacaaacaagaacctctctttctagggatttctaaatatataaaatgactgttccttagaatgtttaacttaagaattatttcagtttgtctgggccacactggggcagaggggggagggagggatacagagatggatgccacttacctcagatcttttaaagtggaaatccaaattgaattttcatttggactttcaggataattttctatgttggtcaacttttcgttttccctaactcacccagtttagtttgggatgatttgatttctgttgttgttgatcccatttctaacttggaattgtgagcctctatgttttctgttaggtgagtgtgttgggttttttccccccaccaggaagtggcagcatccctccttctcccctaaagggactctgcggaacctttcacacctctttctcagggacggggcaggtgtgtgtgtggtacactgacgtgtccagaagcagcactttgactgctctggagtagggttgtacaatttcaaggaatgtttggatttcctgcatcttgtggattactccttagataccgcatagattgcaatataatgctgcatgttcaagatgaacagtagctcctagtaatcataaaatccactctttgcacagtttgatctttactgaaatatgttgccaaaatttatttttgttgttgtagctctggattttgttttgttttgttttttaaggaaacgattgacaataccctttaacatctgtgactactaaggaaacctatttctttcatagagagaaaaatctccaatgcttttgaagacactaataccgtgctatttcagatatgggtgaggaagcagagctctcggtaccgaaggccgggcttcttgagctgtgttggttgtcatggctactgtttcatgaaccacaagcagctcaacagactggtctgttgccttctgaaaccctttgcacttcaatttgcaccaggtgaaaacagggccagcagactccatggcccaattcggtttcttcggtggtgatgtgaaaggagagaattacactttttttttttttaagtggcgtggaggcctttgcttccacatttgtttttaacccagaatttctgaaatagagaatttaagaacacatcaagtaataaatatacagagaatatacttttttataaagcacatgcatctgctattgtgttgggttggtttcctctcttttccacggacagtgttgtgtttctggcatagggaaactccaaacaacttgcacacctctactccggagctgagatttcttttacatagatgacctcgcttcaaatacgttaccttactgatgataggatcttttcttgtagcactataccttgtgggaatttttttttaaatgtacacctgatttgagaagctgaagaaaacaaaattttgaagcactcactttgaggagtacaggtaatgttttaaaaaattgcacaaaagaaaaatgaatgtcgaaatgattcattcagtgtttgaaagatatggctctgttgaaacaatgagtttcatactttgtttgtaaaaaaaaaaaagcagagaagggttgaaagttacatgtttttttgtatatagaaatttgtcatgtctaaatgatcagatttgtatggttatggcctggaagaattactacgtaaaaggctcttaaactatacctatgcttattgttatttttgttacatatagccctcgtctgagggaggggaactcggtattctgcgatttgagaatactgttcattcctatgctgaaagtacttctctgagctcccttcttagtctaaactcttaagccattgcaacttctttttcttcagagatgatgtttgacattttcagcacttcctgttcctataaacccaaagaatataatcttgaacacgaagtgtttgtaacaagggatccaggctaccaatcaaacaggactcattatggggacaaaaaaaaaaattatttcaccttctttccccccacacctcatttaaatggggggagtaaaaacatgatttcaatgtaaatgcctcattttattttagttttattttgatttttatttaatataaagaggccagaataaatacggagcatcttctcagaatagtattcctgtccaaaaatcaagccggacagtggaaactggacagctgtggggatattaagcacccccacttacaattcttaaattcagaatctcgtcccctcccttctcgttgaaggcaactgttctggtagctaactttctcctgtgtaatggcgggagggaacaccggcttcagtttttcatgtccccatgacttgcatacaaatggttcaactgtattaaaattaagtgcatttggccaataggtagtatctatacaataacaacaatctctaagaatttccataacttttcttatctgaaaggactcaagtcttccactgcagatacattggaggcttcacccacgttttctttccctttagtttgtttgctgtctggatggccaatgagcctgtctccttttctgtggccaatctgaaggccttcgttggaagtgttgtttacagtaatccttaccaagataacatactgtcctccagaataccaagtattaggtgacactagctcaagctgttgtcttcagagcagttaccaagaagctcggtgcacaggttttctctggttcttacaggaaccacctactctttcagttttctggcccaggagtggggtaaatcctttagttagtgcatttgaacttgatacctgtgcattcagttctgtgaatactgccctttttggcggggtttcctcatctccccagcctgaactgctcaactctaaacccaaattagtgtcagccgaaaggaggtttcaagatagtcctgtcagtatttgtggtgaccttcagattagacagtcttcatttccagccagtggagtcctggctccagagccatctctgagactcgtactactggatgttttaatatcagatcattacccaccatatgcctcccacaggccaagggaaaacagacaccagaacttgggttgagggcactaccagactgacatggccagtacagaggagaactagggaaggaatgatgttttgcaccttattgaaaagaaaattttaagtgcatacataatagttaagagcttttattgtgacaggagaacttttttccatatgcgtgcatactctctgtaattccagtgtaaaatattgtacttgcactagcttttttaaacaaatattaaaaaatggaagaattcatattctattttctaatcgtggtgtgtctatttgtaggatacactcgagtctgtttattgaattttatggtccctttctttgatggtgcttgcaggttttctaggtagaaattatttcattattataataaaacaatgtttgattcaaaatttgaacaaaattgttttaaataaattgtctgtataccagtacaagtttattgtttcagtatactcgtactaataaaataacagtgccaattgca

SEQ ID NO: 496 - DNA, 13481 bp, Homo sapiens
gctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagctctataaggaaattaaaaagaatggtgcccctcggagtttgcgtgctgccctgtggaggtttgctgagctggctcacctggttcggcctcagaaatgcaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttatagctggagggggttcctcatgcagccctgtcctttcaagaaaacaaaaaggcaaagtgctcttaggagaagaagaagccttggaggatgactctgaatcgagatcggatgtcagcagctctgccttaacagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtgttagacggtaccgacaaccagtatttgggcctgcagattggacagccccaggatgaagatgaggaagccacaggtattcttcctgatgaagcctcggaggccttcaggaactcttccatggcccttcaacaggcacatttattgaaaaacatgagtcactgcaggcagccttctgacagcagtgttgataaatttgtgttgagagatgaagctactgaaccgggtgatcaagaaaacaagccttgccgcatcaaaggtgacattggacagtccactgatgatgactctgcacctcttgtccattgtgtccgccttttatctgcttcgtttttgctaacagggggaaaaaatgtgctggttccggacagggatgtgagggtcagcgtgaaggccctggccctcagctgtgtgggagcagctgtggccctccacccggaatctttcttcagcaaactctataaagttcctcttgacaccacggaataccctgaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggaaatacattttctttggcggattgcattcctttgctgcggaaaacactgaaggatgagtcttctgttacttgcaagttagcttgtacagctgtgaggaactgtgtcatgagtctctgcagcagcagctacagtgagttaggactgcagctgatcatcgatgtgctgactctgaggaacagttcctattggctggtgaggacagagcttctggaaacccttgcagagattgacttcaggctggtgagctttttggaggcaaaagcagaaaacttacacagaggggctcatcattatacagggcttttaaaactgcaagaacgagtgctcaataatgttgtcatccatttgcttggagatgaagaccccagggtgcgacatgttgccgcagcatcactaattaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccagaatatatagaggctataacctactaccaagcataacagacgtcactatggaaaataacctttcaagagttattgcagcagtttctcatgaactaatcacatcaaccaccagagcactcacatttggatgctgtgaagctttgtgtcttctttccactgccttcccagtttgcatttggagtttaggttggcactgtggagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggcagccttgccttctctaacaaaccccccttctctaagtcccatccgacgaaaggggaaggagaaagaaccaggagaacaagcatctgtaccgttgagtcccaagaaaggcagtgaggccagtgcagcttctagacaatctgatacctcaggtcctgttacaacaagtaaatcctcatcactggggagtttctatcatcttccttcatacctcaaactgcatgatgtcctgaaagctacacacgctaactacaaggtcacgctggatcttcagaacagcacggaaaagtttggagggtttctccgctcagccttggatgttctttctcagatactagagctggccacactgcaggacattgggaagtgtgttgaagagatcctaggatacctgaaatcctgctttagtcgagaaccaatgatggcaactgtttgtgttcaacaattgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcgggatggtttgatgtcctccagaaagtgtctacccagttgaagacaaacctcacgagtgtcacaaagaaccgtgcagataagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtgtttattggctttgtattgaaacagtttgaatacattgaagtgggccagttcagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatgccataccggctctgcagcccatagtccacgacctctttgtattaagaggaacaaataaagctgatgcaggaaaagagcttgaaacccaaaaagaggtggtggtgtcaatgttactgagactcatccagtaccatcaggtgttggagatgttcattcttgtcctgcagcagtgccacaaggagaatgaagacaagtggaagcgactgtctcgacagatagctgacatcatcctcccaatgttagccaaacagcagatgcacattgactctcatgaagcccttggagtgttaaatacattatttgagattttggccccttcctccctccgtccggtagacatgcttttacggagtatgttcgtcactccaaacacaatggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtttctattacaactggttggtattcttttagaagacattgttacaaaacagctgaaggtggaaatgagtgagcagcaacatactttctattgccaggaactaggcacactgctaatgtgtctgatccacatcttcaagtctggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaaaagacacagtctgtccagcacaaagttacttagtccccagatgtctggagaagaggaggattctgacttggcagccaaacttggaatgtgcaatagagaaatagtacgaagaggggctctcattctcttctgtgattatgtctgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacagagcagcatggcccagttgccaatggaagaactcaacagaatccaggaataccttcagagcagcgggctcgctcagagacaccaaaggctctattccctgctggacaggtttcgtctctccaccatgcaagactcacttagtccctctcctccagtctcttcccacccgctggacggggatgggcacgtgtcactggaaacagtgagtccggacaaagactggtacgttcatcttgtcaaatcccagtgttggaccaggtcagattctgcactgctggaaggtgcagagctggtgaatcggattcctgctgaagatatgaatgccttcatgatgaactcggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccgttgcagtgcagcctggagagcagcttcttagtccagaaagaaggacaaataccccaaaagccatcagcgaggaggaggaggaagtagatccaaacacacagaatcctaagtatatcactgcagcctgtgagatggtggcagaaatggtggagtctctgcagtcggtgttggccttgggtcataaaaggaatagcggcgtgccggcgtttctcacgccattgctaaggaacatcatcatcagcctggcccgcctgccccttgtcaacagctacacacgtgtgcccccactggtgtggaagcttggatggtcacccaaaccgggaggggattttggcacagcattccctgagatccccgtggagttcctccaggaaaaggaagtctttaaggagttcatctaccgcatcaacacactaggctggaccagtcgtactcagtttgaagaaacttgggccaccctccttggtgtcctggtgacgcagcccctcgtgatggagcaggaggagagcccaccagaagaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgggaggaagctgagcattatcagagggattgtggagcaagagattcaagcaatggtttcaaagagagagaatattgccacccatcatttatatcaggcatgggatcctgtcccttctctgtctccggctactacaggtgccctcatcagccacgagaagctgctgctacagatcaaccccgagcgggagctggggagcatgagctacaaactcggccaggtgtccatacactccgtgtggctggggaacagcatcacacccctgagggaggaggaatgggacgaggaagaggaggaggaggccgacgcccctgcaccttcgtcaccacccacgtctccagtcaactccaggaaacaccgggctggagttgacatccactcctgttcgcagtttttgcttgagttgtacagccgctggatcctgccgtccagctcagccaggaggaccccggccatcctgatcagtgaggtggtcagatcccttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccactgcgtgaacattcacagccagcagcacgtactggtcatgtgtgccactgcgttttacctcattgagaactatcctctggacgtagggccggaattttcagcatcaataatacagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggaaaggagaaagtcagtccgggtagaacttcagaccctaatcctgcagcccccgacagcgagtcagtgattgttgctatggagcgggtatctgttctttttgataggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgcgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgtaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcacctgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggccaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattccgtattggttgggggctcctgtttctcatcctagctttttcctggaaagcccgctagaaggtttgggaacgaggggaaagttctcagaactgttggctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctgggcctcctgggaaggagggagctgctcagaatgccgcatgacaactgaaggcaacctggaaggttcaggggccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgtccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaaggaaaaaaatggaaaccatca

SEQ ID NO: 497 - DNA, 5850 bp, Homo sapiens
acaagtctttccgcctccccagcccgcccgggagctgcgagccgcgagctggattatggtggcctgagcagccaacgcagccgcaggagcccggagcccttgcccctgcccgcgccgccgcccgccggggggaccagggaagccgccaccggcccgccatgcccgcccctcccagccccgccgggagcccgcgcccgctgcccaggctggccgccgccgtgccgatgtagcgggctccggatcccagcctctcccctgctcccgtgctctgcggatctcccctgaccgctctccacagcccggacccgggggctggcccagggccctgcaggccctggcgtcctgatgcccccaagctccctctcctgagaagccaccagcaccacccagacttgggggcaggcgccagggacggacgtgggccagtgcgagcccagagggcccgaaggccggggcccaccatggcccaagccctgccctggctcctgctgtggatgggcgcgggagtgctgcctgcccacggcacccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaaccctcatgaccatagcctatgtcatggctgccatctgcgccctcttcatgctgccactctgcctcatggtgtgtcagtggcgctgcctccgctgcctgcgccagcagcatgatgactttgctgatgacatctccctgctgaagtgaggaggcccatgggcagaagatagagattcccctggaccacacctccgtggttcactttggtcacaagtaggagacacagatggcacctgtggccagagcacctcaggaccctccccacccaccaaatgcctctgccttgatggagaaggaaaaggctggcaaggtgggttccagggactgtacctgtaggaaacagaaaagagaagaaagaagcactctgctggcgggaatactcttggtcacctcaaatttaagtcgggaaattctgctgcttgaaacttcagccctgaacctttgtccaccattcctttaaattctccaacccaaagtattcttcttttcttagtttcagaagtactggcatcacacgcaggttaccttggcgtgtgtccctgtggtaccctggcagagaagagaccaagcttgtttccctgctggccaaagtcagtaggagaggatgcacagtttgctatttgctttagagacagggactgtataaacaagcctaacattggtgcaaagattgcctcttgaattaaaaaaaaaaactagattgactatttatacaaatgggggcggctggaaagaggagaaggagagggagtacaaagacagggaatagtgggatcaaagctaggaaaggcagaaacacaaccactcaccagtcctagttttagacctcatctccaagatagcatcccatctcagaagatgggtgttgttttcaatgttttcttttctgtggttgcagcctgaccaaaagtgagatgggaagggcttatctagccaaagagctcttttttagctctcttaaatgaagtgcccactaagaagttccacttaacacatgaatttctgccatattaatttcattgtctctatctgaaccaccctttattctacatatgataggcagcactgaaatatcctaaccccctaagctccaggtgccctgtgggagagcaactggactatagcagggctgggctctgtcttcctggtcataggctcactctttcccccaaatcttcctctggagctttgcagccaaggtgctaaaaggaataggtaggagacctcttctatctaatccttaaaagcataatgttgaacattcattcaacagctgatgccctataacccctgcctggatttcttcctattaggctataagaagtagcaagatctttacataattcagagtggtttcattgccttcctaccctctctaatggcccctccatttatttgactaaagcatcacacagtggcactagcattataccaagagtatgagaaatacagtgctttatggctctaacattactgccttcagtatcaaggctgcctggagaaaggatggcagcctcagggcttccttatgtcctccaccacaagagctccttgatgaaggtcatctttttcccctatcctgttcttcccctccccgctcctaatggtacgtgggtacccaggctggttcttgggctaggtagtggggaccaagttcattacctccctatcagttctagcatagtaaactacggtaccagtgttagtgggaagagctgggttttcctagtatacccactgcatcctactcctacctggtcaacccgctgcttccaggtatgggacctgctaagtgtggaattacctgataagggagagggaaatacaaggagggcctctggtgttcctggcctcagccagctgcccacaagccataaaccaataaaacaagaatactgagtcagttttttatctgggttctcttcattcccactgcacttggtgctgctttggctgactgggaacaccccataactacagagtctgacaggaagactggagactgtccacttctagctcggaacttactgtgtaaataaactttcagaactgctaccatgaagtgaaaatgccacattttgctttataatttctacccatgttgggaaaaactggctttttcccagccctttccagggcataaaactcaaccccttcgatagcaagtcccatcagcctattatttttttaaagaaaacttgcacttgtttttctttttacagttacttccttcctgccccaaaattataaactctaagtgtaaaaaaaagtcttaacaacagcttcttgcttgtaaaaatatgtattatacatctgtatttttaaattctgctcctgaaaaatgactgtcccattctccactcactgcatttggggcctttcccattggtctgcatgtcttttatcattgcaggccagtggacagagggagaagggagaacaggggtcgccaacacttgtgttgctttctgactgatcctgaacaagaaagagtaacactgaggcgctcgctcccatgcacaactctccaaaacacttatcctcctgcaagagtgggctttccagggtctttactgggaagcagttaagccccctcctcaccccttccttttttctttctttactcctttggcttcaaaggattttggaaaagaaacaatatgctttacactcattttcaatttctaaatttgcaggggatactgaaaaatacggcaggtggcctaaggctgctgtaaagttgaggggagaggaaatcttaagattacaagataaaaaacgaatcccctaaacaaaaagaacaatagaactggtcttccattttgccacctttcctgttcatgacagctactaacctggagacagtaacatttcattaaccaaagaaagtgggtcacctgacctctgaagagctgagtactcaggccactccaatcaccctacaagatgccaaggaggtcccaggaagtccagctccttaaactgacgctagtcaataaacctgggcaagtgaggcaagagaaatgaggaagaatccatctgtgaggtgacaggcaaggatgaaagacaaagaaggaaaagagtatcaaaggcagaaaggagatcatttagttgggtctgaaaggaaaagtctttgctatccgacatgtactgctagtacctgtaagcattttaggtcccagaatggaaaaaaaaatcagctattggtaatataataatgtcctttccctggagtcagtttttttaaaaagttaactcttagtttttacttgtttaattctaaaagagaagggagctgaggccattccctgtaggagtaaagataaaaggataggaaaagattcaaagctctaatagagtcacagctttcccaggtataaaacctaaaattaagaagtacaataagcagaggtggaaaatgatctagttcctgatagctacccacagagcaagtgatttataaatttgaaatccaaactactttcttaatatcactttggtctccatttttcccaggacaggaaatatgtccccccctaactttcttgcttcaaaaattaaaatccagcatcccaagatcattctacaagtaattttgcacagacatctcctcaccccagtgcctgtctggagctcacccaaggtcaccaaacaacttggttgtgaaccaactgccttaaccttctgggggagggggattagctagactaggagaccagaagtgaatgggaaagggtgaggacttcacaatgttggcctgtcagagcttgattagaagccaagacagtggcagcaaaggaagacttggcccaggaaaaacctgtgggttgtgctaatttctgtccagaaaatagggtggacagaagcttgtggggtacatggaggaattgggacctggttatgttgttattctcggactgtgaattttggtgatgtaaaacagaatattctgtaaacctaatgtctgtataaataatgagcgttaacacagtaaaatattcaataagaagtcaaaaaaaaaaaaaaaaaa

SEQ ID NO: 498 - DNA, 3215 bp, Homo sapiens
aggagaaggagaaggaggaggactaggaggaggaggacggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 499 - DNA, 981 bp, Homo sapiens
gtttggggccagagtgggcgaggcgcggaggtctggcctataaagtagtcgcggagacggggtgctggtttgcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaatgacctgtattttgccagacttaaatcacagatgggtattaaacttgtcagaatttctttgtcattcaagcctgtgaataaaaaccctgtatggcacttattatgaggctattaaaagaatccaaattcaaactaaaaaaaaaaaaaaaaaa

SEQ ID NO: 500 - DNA, 1669 bp, Homo sapiens
ctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaa

SEQ ID NO: 501 - DNA, 1498 bp, Homo sapiens
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 502 - DNA, 9469 bp, Homo sapiens
ctcttatgtgaggagctgaagaggaattaaaatatacaggatgaaaagatggcaatgttgcctcccccaggacctcagagctttgtccatttcacaaaacagtctcttgccctcattgaacaacgcattgctgaaagaaaatcaaaggaacccaaagaagaaaagaaagatgatgatgaagaagccccaaagccaagcagtgacttggaagctggcaaacagctgcccttcatctatggggacattcctcccggcatggtgtcagagcccctggaggacttggacccctactatgcagacaaaaagactttcatagtattgaacaaagggaaaacaatcttccgtttcaatgccacacctgctttatatatgctttctcctttcagtcctctaagaagaatatctattaagattttagtacactccttattcagcatgctcatcatgtgcactattctgacaaactgcatatttatgaccatgaataacccaccggactggaccaaaaatgtcgagtacacttttactggaatatatacttttgaatcacttgtaaaaatccttgcaagaggcttctgtgtaggagaattcacttttcttcgtgacccgtggaactggctggattttgtcgtcattgtttttgcgtatttaacagaatttgtaaacctaggcaatgtttcagctcttcgaactttcagagtattgagagctttgaaaactatttctgtaatcccaggcctgaagacaattgtaggggctttgatccagtcagtgaagaagctttctgatgtcatgatcctgactgtgttctgtctgagtgtgtttgcactaattggactacagctgttcatgggaaacctgaagcataaatgttttcgaaattcacttgaaaataatgaaacattagaaagcataatgaataccctagagagtgaagaagactttagaaaatatttttattacttggaaggatccaaagatgctctcctttgtggtttcagcacagattcaggtcagtgtccagaggggtacacctgtgtgaaaattggcagaaaccctgattatggctacacgagctttgacactttcagctgggccttcttagccttgtttaggctaatgacccaagattactgggaaaacctttaccaacagacgctgcgtgctgctggcaaaacctacatgatcttctttgtcgtagtgattttcctgggctccttttatctaataaacttgatcctggctgtggttgccatggcatatgaagaacagaaccaggcaaacattgaagaagctaaacagaaagaattagaatttcaacagatgttagaccgtcttaaaaaagagcaagaagaagctgaggcaattgcagcggcagcggctgaatatacaagtattaggagaagcagaattatgggcctctcagagagttcttctgaaacatccaaactgagctctaaaagtgctaaagaaagaagaaacagaagaaagaaaaagaatcaaaagaagctctccagtggagaggaaaagggagatgctgagaaattgtcgaaatcagaatcagaggacagcatcagaagaaaaagtttccaccttggtgtcgaagggcataggcgagcacatgaaaagaggttgtctacccccaatcagtcaccactcagcattcgtggctccttgttttctgcaaggcgaagcagcagaacaagtctttttagtttcaaaggcagaggaagagatataggatctgagactgaatttgccgatgatgagcacagcatttttggagacaatgagagcagaaggggctcactgtttgtgccccacagaccccaggagcgacgcagcagtaacatcagccaagccagtaggtccccaccaatgctgccggtgaacgggaaaatgcacagtgctgtggactgcaacggtgtggtctccctggttgatggacgctcagccctcatgctccccaatggacagcttctgccagagggcacgaccaatcaaatacacaagaaaaggcgttgtagttcctatctcctttcagaggatatgctgaatgatcccaacctcagacagagagcaatgagtagagcaagcatattaacaaacactgtggaagaacttgaagagtccagacaaaaatgtccaccttggtggtacagatttgcacacaaattcttgatctggaattgctctccatattggataaaattcaaaaagtgtatctattttattgtaatggatccttttgtagatcttgcaattaccatttgcatagttttaaacacattatttatggctatggaacaccacccaatgactgaggaattcaaaaatgtacttgctataggaaatttggtctttactggaatctttgcagctgaaatggtattaaaactgattgccatggatccatatgagtatttccaagtaggctggaatatttttgacagccttattgtgactttaagtttagtggagctctttctagcagatgtggaaggattgtcagttctgcgatcattcagactgctccgagtcttcaagttggcaaaatcctggccaacattgaacatgctgattaagatcattggtaactcagtaggggctctaggtaacctcaccttagtgttggccatcatcgtcttcatttttgctgtggtcggcatgcagctctttggtaagagctacaaagaatgtgtctgcaagatcaatgatgactgtacgctcccacggtggcacatgaacgacttcttccactccttcctgattgtgttccgcgtgctgtgtggagagtggatagagaccatgtgggactgtatggaggtcgctggtcaagctatgtgccttattgtttacatgatggtcatggtcattggaaacctggtggtcctaaacctatttctggccttattattgagctcatttagttcagacaatcttacagcaattgaagaagaccctgatgcaaacaacctccagattgcagtgactagaattaaaaagggaataaattatgtgaaacaaaccttacgtgaatttattctaaaagcattttccaaaaagccaaagatttccagggagataagacaagcagaagatctgaatactaagaaggaaaactatatttctaaccatacacttgctgaaatgagcaaaggtcacaatttcctcaaggaaaaagataaaatcagtggttttggaagcagcgtggacaaacacttgatggaagacagtgatggtcaatcatttattcacaatcccagcctcacagtgacagtgccaattgcacctggggaatccgatttggaaaatatgaatgctgaggaacttagcagtgattcggatagtgaatacagcaaagtgagattaaaccggtcaagctcctcagagtgcagcacagttgataaccctttgcctggagaaggagaagaagcagaggctgaacctatgaattccgatgagccagaggcctgtttcacagatggttgtgtacggaggttctcatgctgccaagttaacatagagtcagggaaaggaaaaatctggtggaacatcaggaaaacctgctacaagattgttgaacacagttggtttgaaagcttcattgtcctcatgatcctgctcagcagtggtgccctggcttttgaagatatttatattgaaaggaaaaagaccattaagattatcctggagtatgcagacaagatcttcacttacatcttcattctggaaatgcttctaaaatggatagcatatggttataaaacatatttcaccaatgcctggtgttggctggatttcctaattgttgatgtttctttggttactttagtggcaaacactcttggctactcagatcttggccccattaaatcccttcggacactgagagctttaagacctctaagagccttatctagatttgaaggaatgagggtcgttgtgaatgcactcataggagcaattccttccatcatgaatgtgctacttgtgtgtcttatattctggctgatattcagcatcatgggagtaaatttgtttgctggcaagttctatgagtgtattaacaccacagatgggtcacggtttcctgcaagtcaagttccaaatcgttccgaatgttttgcccttatgaatgttagtcaaaatgtgcgatggaaaaacctgaaagtgaactttgataatgtcggacttggttacctatctctgcttcaagttgcaacttttaagggatggacgattattatgtatgcagcagtggattctgttaatgtagacaagcagcccaaatatgaatatagcctctacatgtatatttattttgtcgtctttatcatctttgggtcattcttcactttgaacttgttcattggtgtcatcatagataatttcaaccaacagaaaaagaagcttggaggtcaagacatctttatgacagaagaacagaagaaatactataatgcaatgaaaaagctggggtccaagaagccacaaaagccaattcctcgaccagggaacaaaatccaaggatgtatatttgacctagtgacaaatcaagcctttgatattagtatcatggttcttatctgtctcaacatggtaaccatgatggtagaaaaggagggtcaaagtcaacatatgactgaagttttatattggataaatgtggtttttataatccttttcactggagaatgtgtgctaaaactgatctccctcagacactactacttcactgtaggatggaatatttttgattttgtggttgtgattatctccattgtaggtatgtttctagctgatttgattgaaacgtattttgtgtcccctaccctgttccgagtgatccgtcttgccaggattggccgaatcctacgtctagtcaaaggagcaaaggggatccgcacgctgctctttgctttgatgatgtcccttcctgcgttgtttaacatcggcctcctgctcttcctggtcatgttcatctacgccatctttggaatgtccaactttgcctatgttaaaaaggaagatggaattaatgacatgttcaattttgagacctttggcaacagtatgatttgcctgttccaaattacaacctctgctggctgggatggattgctagcacctattcttaacagtaagccacccgactgtgacccaaaaaaagttcatcctggaagttcagttgaaggagactgtggtaacccatctgttggaatattctactttgttagttatatcatcatatccttcctggttgtggtgaacatgtacattgcagtcatactggagaattttagtgttgccactgaagaaagtactgaacctctgagtgaggatgactttgagatgttctatgaggtttgggagaagtttgatcccgatgcgacccagtttatagagttctctaaactctctgattttgcagctgccctggatcctcctcttctcatagcaaaacccaacaaagtccagctcattgccatggatctgcccatggttagtggtgaccggatccattgtcttgacatcttatttgcttttacaaagcgtgttttgggtgagagtggggagatggattctcttcgttcacagatggaagaaaggttcatgtctgcaaatccttccaaagtgtcctatgaacccatcacaaccacactaaaacggaaacaagaggatgtgtctgctactgtcattcagcgtgcttatagacgttaccgcttaaggcaaaatgtcaaaaatatatcaagtatatacataaaagatggagacagagatgatgatttactcaataaaaaagatatggcttttgataatgttaatgagaactcaagtccagaaaaaacagatgccacttcatccaccacctctccaccttcatatgatagtgtaacaaagccagacaaagagaaatatgaacaagacagaacagaaaaggaagacaaagggaaagacagcaaggaaagcaaaaaatagagcttcatttttgatatattgtttacagcctgtgaaagtgatttatttgtgttaataaaactcttttgaggaagtctatgccaaaatcctttttatcaaaatattctcgaaggcagtgcagtcactaactctgatttcctaagaaaggtgggcagcattagcagatggttatttttgcactgatgattctttaagaatcgtaagagaactctgtaggaattattgattatagcatacaaaagtgattcagttttttggtttttaataaatcagaagaccatgtagaaaacttttacatctgccttgtcatcttttcacaggattgtaattagtcttgtttcccatgtaaataaacaacacacgcatacagaaaaatctattatttatctattatttggaaatcaacaaaagtatttgccttggctttgcaatgaaatgcttgatagaagtaatggacattagttatgaatgtttagttaaaatgcattattagggagcttgactttttatcaatgtacagaggttattctatattttgaggtgcttaaatttattctacattgcatcagaaccaatttatatgtgcctataaaatgccatgggattaaaaatatatgtaggctattcatttctacaaatgtttttcattcatcttgactcacatgccaacaaggataagacttacctttagagtattgtgtttcatagcctttcttctttcatatccctttttgttcatagaataaccacagaacttgaaaaattattctaagtacatattacactcctcaaaaaaaacaaagataactgagaaaaaagttattgacagaagttctatttgctattatttacatagcctaacatttgactgtgctgcccaaaatactgataatagtctcttaaactcttttgtcaaattttcctgctttcttatgcagtattgtttagtcatcctttcgctgtaagcaaagttgatgaaatccttcctgatatgcagttagttgtttgaccacggtacatacttgagcagataataacttgggcacagtatttattgcatcacttgtatacaatcccgtgtttggcaagctttcaaatcatgtaatatgacagactttacacagatatgtgtttagtatgaataaaaaagcattgaaatagggattcttgccaacttgctctcttgccaccaacttactttcctaaattatggaagtaatcttttttggatatacttcaatgtatacaatgaggaagatgtcaccttctccttaaaattctatgatgtgaaatatattttgcctcaatcaacacagtaccatgggcttctaatttatcaagcacatattcattttgcattagctgtagacatctagttttttgaaaacacctattaatagtaatttgaaaagaaataaccataatgctttttttcgtgagtttatttcaggaatatgagatctttcttctataaagttattcatgcacaggcaaaaattgagctacacaggtagaatgtagttttacttagaagatttttgtgggaggttttgaagcaaatatataaaacaactttcactaatttgctttccatatttaaaaaataataaattacatttatataataaatgtttaaagcacatattttttgttgttctggcaatttaaaaagaaagaggatttaaacgtacctatagaaacaaagatttatggttaaagaatgagatcagaagtctagaatgtttttaaattgtgatatattttacaacatccgttattactttgagacatttgtcctaatctacgtataaaactcaatctagggctaaagattctttataccatcttaggttcattcatcttaggctatttgaaccactttttaatttaatatgaaagacaccatgcagtgttttccgagactacatagatcattttatcacatacctaccaagcctgttggaaataggttttgataatttaagtagggacctatacaaaatatattacatttatcagatttttaaatacattcaattaagaatttaacatcaccttaaatttgaattcaatctaccgttatttcaaactcacaaatataactgcattatgaatacttacataatgtagtaagacaagatgtttgacaggttcgtgtgtaattttctattaatgtttttacattgccttgtttttatgtaaaataaaaaatatgggcaactggtttgttaacaacacaatttcttcttagcatttcaaaaatatatataaagttgttctttttcctatttcatgaactatgtttttttttaaaataacatggttaagttttatatatatttacgtttgtttcaggaatgtctacttgtgactttttatcaattaaaaataatatttggaagaaagagcttattaagtataagcttgaagtaaaattagacctctctttccatgtagattactgtttgtactgatggtttcacccttcagaaggcactgtcatattaatatttaaattttataatcgctgaacttattacacccaacaatacagaaaggcagttacactgaagaacttaacttagaataaaatggaagcaaacaggttttctaaaaacttttttaagtgaccaggtctcgctctgtcacccaggctagagtgcaatggcatgatcatagctctctgcagcctcaactctgggctcaagcaaccctcctgcctcagcctcccaagtagctaagactacaggtacatgccaccatgcctggctaatatttaaatttttgtagataaggggtcttgctatgttgcccaggctagtctcaaactcctggcttcaagtgttcctactgtcatgacctgccaacatgctggggttacaggcatgagccaccatgccccaaacaggtttgaacacaaatctttcggatgaaaattagagaacctaattttagctttttgatagttacctagtttgcaaaagatttgggtgacttgtgagctgtttttaaatgctgattgttgaacatcacaacccaaaatacttagcatgattttatagagttttgatagctttattaaaaagagtgaaaataaaatgcatatgtaaataaagcagttctaaatagctatttcagagaaatgttaatagaagtgctgaaagaagggccaactaaattaggatggccagggaattggcctgggtttaggacctatgtatgaaggccaccaattttttaaaaatatctgtggtttattatgttattatcttcttgaggaaaacaatcaagaattgcttcatgaaaataaataaatagccatgaatatcataaagctgtttacataggattctttacaaatttcatagatctatgaatgctcaaaatgtttgagtttgccataaattatattgtagttatattgtagttatacttgagactgacacattgtaatataatctaagaataaaagttatacaaaataaaa

SEQ ID NO: 503 - DNA, 3916 bp, Homo sapiens
gagcaggaagtgtttgaggaagtcgcgccgcgctgcccgcgttaagattcccgcattttaatgttttcaggggggtgtcatagccccgggtttggccgccccagccccgccttccccgccccggggagcccgccccctgccccgcgtccctgccgacagagttagcacgacatcagtatgagctggtcaccttccctgacaacgcagacatgtggggcctgggaaatgaaagagcgccttgggacagggggatttggaaatgtcatccgatggcacaatcaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatgatgtggggggactcgaccccctgacatggggcagcccatagcaggccttgtgcagtggggggactcgaccccctgacatggggctgcctggagcaggccgcgtgacgtggggctgcctggccgcggctctcacatggtggttcctgctgcactgatggcccaggggtctctggtatccagatggagctctcgcttcctcagcagctgtgactttcacccaggacccaggacgcagccctccgtgggcactgccggcgccttgtctgcacactggaggtcctccattacagaggcccagcgcacatcgctggccccacaaacgttcaggggtacagccatggcagctccttcctctgccgtgagaaaagtgcttggagtacggtttgccacacacgtgactggacagtgtccaattcaaatctttcagggcagagtccgagcagcgcttggtgacagcctgtcctctcctgctctccaaaggccctgctccctgtcctctctcactttacagcttgtgtttcttctggattcagcttctcctaaacagacagtttaattatagttgcggcctggccccatcctcacttcctctttttatttcactgctgctaaaattgtgtttttacctactactttggtggttgtcctcttttcggcaaagttggagcgagtgccaagctctccatctgtggtcctttctgccaagagcgactcatagtaaccaggatgggagagcagctgccttattctgaatcccaaaaattacttgggggtgattgtcacagaggagggacagaaagggtatctgctgaccaccagcctgcctacccatgcccatgtctccattcctgctcaagcgtgtgtgctgggccggggagtccctgtctctcacagcatctagcagtattattaaatggattcattttaaaaatagctcctatattttgtaacatgtctcaaacactcatactgggttccacaatccactgttagaatacctatggttagggcttctgaactaaaataatggaaaattttaacaatttgtatagtgcctggatcattactagtgccataaccctgcttcttcaacatttcacagaacttctcttttatataaaggcaagagcacaaaatgagttcagatgatcacaaacaggtgagttttgttggagaagaaagttggagtaggagactttcacaagtggtttccatggagatagaatgaagcattctgtggtcaagtaagtttagggagctattcatgtttcacttgctttgtggagattcacactatgcactgggaaagtatctgaaaagtcttataataaagaaacaggcttaactttgtgtaagaacactgtttatcaatgtcatttggctatagaaacattttctcctgctgattgtgtgtgtgaaacatgtattaacattccaatgaactagcatttaataaagcacaattttgg

SEQ ID NO: 504 - DNA, 2595 bp, Homo sapiens
agcgcgcaggcgcggccggattccgggcagtgacgcgacggcgggccgcgcggcgcatttccgcctctggcgaatggctcgtctgtagtgcacgccgcgggcccagctgcgaccccggccccgcccccgggaccccggccatggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagttcctatagaagagcagcgtggggactacgacctgaatgctgtgcggctctgcttccaggtgacagtgcgggacccatcaggcaggcccctccgcctgccgcctgtcctttctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactgggttgcaggggattgaagccctccaaaagcacttacggattctggtggggtgtgttccaactgcccccaactttgtggatgtcttccttggaggggggagccatattttattcttttattgtcagtatctgtatctctctctctttttggaggtgcttaagcagaagcattaacttctctggaaaggggggagctggggaaactcaaacttttcccctgtcctgatggtcagctcccttctctgtagggaactctggggtcccccatccccatcctccagcttctggtactctcctagagacagaagcaggctggaggtaaggcctttgagcccacaaagccttatcaagtgtcttccatcatggattcattacagcttaatcaaaataacgccccagataccagcccctgtatggcactggcattgtccctgtgcctaacaccagcgtttgaggggctggccttcctgccctacagaggtctctgccggctctttccttgctcaaccatggctgaaggaaaccagtgcaacagcactggctctctccaggatccagaaggggtttggtctgggacttccttgctctccctcttctcaagtgccttaatagtagggtaagttgttaagagtgggggagagcaggctggcagctctccagtcaggaggcatagtttttactgaacaatcaaagcacttggactcttgctctttctactctgaactaataaatctgttgccaagctggctagaaaaaaaaaaaaaaaaaa

SEQ ID NO: 505 - DNA, 2120 bp, Homo sapiens
agcccgttcctgctccgcgcttctggagcactggccaaggcgggccgattcaggacccaggttacttgggcggcgagctggactgtttctactcctccctcctcctccactgcggggtctgaccctactccttgtgtgaggactcctctagttcagagacatattctgttcaccaaacttgactgcgctctatcgaggtcgttaaattcttcggaaatgcctcacatatagtttggcagctagcccttgccctgttggatgaataggcacctctggaagagccaactgtgtgagatggtgcagcccagtggtggcccggcagcagatcaggacgtactgggcgaagagtctcctctggggaagccagccatgctgcacctgccttcagaacagggcgctcctgagaccctccagcgctgcctggaggagaatcaagagctccgagatgccatccggcagagcaaccagattctgcgggagcgctgcgaggagcttctgcatttccaagccagccagagggaggagaaggagttcctcatgtgcaagttccaggaggccaggaaactggtggagagactcggcctggagaagctcgatctgaagaggcagaaggagcaggctctgcgggaggtggagcacctgaagagatgccagcagcagatggctgaggacaaggcctctgtgaaagcccaggtgacgtccttgctcggggagctgcaggagagccagagtcgcttggaggctgccactaaggaatgccaggctctggagggtcgggcccgggcggccagcgagcaggcgcggcagctggagagtgagcgcgaggcgctgcagcagcagcacagcgtgcaggtggaccagctgcgcatgcagggccagagcgtggaggccgcgctccgcatggagcgccaggccgcctcggaggagaagaggaagctggcccagttgcaggtggcctatcaccagctcttccaagaatacgacaaccacatcaagagcagcgtggtgggcagtgagcggaagcgaggaatgcagctggaagatctcaaacagcagctccagcaggccgaggaggccctggtggccaaacaggaggtgatcgataagctgaaggaggaggccgagcagcacaagattgtgatggagaccgttccggtgctgaaggcccaggcggatatctacaaggcggacttccaggctgagaggcaggcccgggagaagctggccgagaagaaggagctcctgcaggagcagctggagcagctgcagagggagtacagcaaactgaaggccagctgtcaggagtcggccaggatcgaggacatgaggaagcggcatgtcgaggtctcccaggcccccttgccccccgcccctgcctacctctcctctcccctggccctgcccagccagaggaggagcccccccgaggagccacctgacttctgctgtcccaagtgccagtatcaggcccctgatatggacaccctgcagatacatgtcatggagtgcattgagtagggccggccagtgcaaggccactgcctgccgaggacgtgcccgggaccgtgcagtctgcgctttcctctcccgcctgcctagcccaggatgaagggctgggtggccacaactgggatgccacctggagccccacccaggagctggccgcggcaccttacgcttcagctgttgatccgctggtcccctcttttggggtagatgcggccccgatcaggcctgactcgctgctctttttgttcccttctgtctgctcgaaccacttgcctcgggctaatccctccctcttcctccacccggcactggggaagtcaagaatggggcctggggctctcagggagaactgcttcccctggcagagctgggtggcagctcttcctcccaccggacaccgacccgcccgctgctgtgccctgggagtgctgccctcttaccatgcacacgggtgctctccttttgggctgcatgctattccattttgcagccagaccgatgtgtatttaaccagtcactattgatggacatttgggttgtttcccatctttttgttaccataaataatggcatagtaaaaatccttgtgcattaaaaaaaaaaaaaaaaaa

SEQ ID NO: 506 - DNA, 1669 bp, Homo sapiens; identical to SEQ ID NO: 500

SEQ ID NO: 507 - DNA, 722 bp, Homo sapiens
acttcgctccgcgcagccgcctggtctgcagtttgttggagctctgcgtccagcgccgctgccgctgccgccgccgccgccgccgccgccgccgccgccgccgccgccactaccaccacttgattcttgcagccaccctgcgaaccctgccacactgcgatcgcatcatcgcggtattcggttcgctgcgttcccgccgccaccgcctcggcgccctttcttggcccttgttcccccaaatgtctgactctgactctcggactgagaaacgcaagaaaaaaagaccaaatggcaaagcaaccttctgatgtaagttctgagtgtgaccgagaaggtagacaattgcagcctgcggagaggcctccccagctcagacctggggcccctacctccctacagacagagccacaagacaggagcccagcacccatgagttgtgacaaatcaacacaaaccccaagtcctccttgccaggccttcaaccactatctcagtgcaatggtagtcatcctagaggatataggtgatctttcactgtgctttggatttatatttactggcttagatttgtatggccaccaccatagtcaagatacagaacaactcaaccacaaggatttctcatgatacctttttatagccacagccacctctctccctcttccttgagcattttgtcatatggtcattggtgattaaataaaatgtattttaatattgac

(19) United States
(12) Patent Application Publication
(10) Pub. No.: US 2010/0298697 A1
(43) Pub. Date: Nov. 25, 2010

(54) METHOD AND DEVICES FOR IMPROVED EFFICIENCY OF RNA DELIVERY TO CELLS

(75) Inventors: DEEPAK Ramesh THAKKER, BLAINE, MN (US); CARL A. SHU, PLYMOUTH, MN (US); ERIC NEAL BURRIGHT, EAGAN, MN (US); PAUL W. WACNIK, MINNEAPOLIS, MN (US); JENNIFER M. HEISEL, PRINCETON, MN (US); LOTHAR KRINKE, EDEN PRAIRIE, MN (US)

Correspondence Address: Kenneth J. Collier - c/o Kathy Illg;Medtronic, Inc., 710 Medtronic Parkway NE, Mail Stop LC-340, Minneapolis, MN, 55432, US

(73) Assignee: MEDTRONIC, INC., MINNEAPOLIS, MN (US)

(21) Appl. No.: 12/782,274

(22) Filed: May 18, 2010

Related U.S. Application Data
(63) Continuation-in-part of application No. 12/468,685, filed on May 19, 2009.

(51) Int. Cl. A61M 37/00; C12N 15/11; A61B 6/00; A61N 1/30; A61N 1/36

(52) U.S. Cl. 600/431; 435/446; 607/3; 604/21

(57) ABSTRACT

The instant invention provides a method for improving efficiency of RNA delivery to cells. The method comprises applying a low strength electric field to the cells and then after a certain time period, administering the ribonucleic acid sequence to the cells. Devices, kits, and RNA molecules suitable for delivery and devices suitable for practicing the disclosed methods are also provided.

### RELATIONSHIP TO PRIOR APPLICATIONS

The instant invention is a continuation-in-part application of U.S. patent application Ser. No. 12/468,685 filed on May 19, 2009 and incorporated herein by reference.

### FIELD OF THE INVENTION

The instant invention is directed to a method of delivering RNA to a cell of a patient, comprising electrically stimulating an area in a patient's body containing said cell, with a low-voltage electric stimulation followed by delivery of the RNA.

### BACKGROUND OF THE INVENTION

RNA interference (RNAi) is a natural method of gene silencing in plant and mammalian cells. RNAi provides a mechanism for the sequence specific silencing of genes. RNAi has been adopted by researchers as a tool to investigate gene function and it has potential applications in the clinical arena such as treatment of neurodegenerative diseases, heart diseases, cancer, and other diseases where silencing of a specific gene or genes is desirable.

However, the efficient delivery of RNAi agents has been a major roadblock on the way to establish RNAi as a suitable gene therapy agent in modern medicine, mostly due to inefficient intake of RNAi agents and quick degradation thereof by RNAses present in blood, lymph, CSF, and intercellular space.

Initial work in the electroporation of siRNAs recommends conditions for transfecting cells in vitro have been to recommend pulse length to 100 μs and pulse voltages from 150-900 V (i.e., 150, 300 . . . 900 V) and then directly transferring the transfected cells to a growth medium(siRNA transfection protocol, Ambion, 2008). Bio-Rad, a maker of in vitro electroportation devices, has published recommended conditions between 200 and 300 volts as optimal for transfection followed by transferring to the cell growth medium (BioTechniques Protocol Guide 2009 (p. 19) doi 10.2144/000113012).

In-Vivo methods for electroporation of RNA to patients has also been described in the literature. In vivo two different electroporation procedures are being used in ongoing clinical trials. In the first procedure, DNA is injected (by needle and syringe) followed by insertion of a four-needle electrode array at the site of injection to deliver electrical pulses, and in the second procedure two standard syringes with injection needles are mounted on a movable sled. As the needles are advanced into the muscle tissue, DNA is injected at a predetermined rate. When DNA injection is completed, electrical pulses are delivered via the two injection needles now serving as electrodes. These clinical studies are sponsored separately by Southampton University Hospitals, and Merck.

Three electroporation devices are known to be approved for use in clinical trials; however, none of these devices are presently commercially available (S. Li (ed.), Electroporation Protocols: Preclinical and Clinical Gene Medicine. 497. From Methods in Molecular Biology, Vol. 423. Humana Press 2008). The first system, the Elgen system, consists of a square wave pulse generator, interfacing with a combined injection/electrode device, which injects the DNA during needle insertion and uses of-the-shelf syringes and needles. The output pulses used in human studies so far were set at a constant current of 250 mA, corresponding to about 60-70 V. The second system, the MedPulser DNA Delivery System (DDS) made by Inovio Biomedical Corporation consists of a pulse generator and a reusable applicator with a disposable tip containing a four-needle array electrode. The MedPulser DDS delivers two unipolar pulses of 60 ms at 106 V, with a frequency of 4 Hz. Typically, DNA vaccine is injected intramuscularly, followed by insertion of the electrode array encompassing the injection site and subsequent pulse delivery. The third system, also made by MedPulser, is the DNA Electroporation Therapy System, and also supplied by Inovio Biomedical Corporation, is similar to the MedPulser DDS. However, it uses a six-needle electrode array, with the needles either integrated into the applicator or contained in a disposable tip (needle length up to 3 cm; electrode distance, 8.6 mm). This system delivers six bipolar, rotating pulses of 100 μs each at 1,130 V, with a frequency of 4 Hz.

So far there does not appear to be any current human studies directed to delivery of RNA molecules subsequent to electroporation. Further the RNA protocol methods are directed to use of relative higher voltages to achieve transfection of these molecules. The present invention overcomes several limitations of the art by providing more efficient delivery of RNAs that does not require the high voltages presently used, which may require sedation during the electroporation process. Further, current electroporation procedures do not provide for chronic delivery of RNA agents. As discussed previously, current delivery protocols couple the nucleic acid delivery with providing the siRNA.

Other delivery methods also have their drawbacks. For example, viral delivery is unproven as an effective in vivo delivery mechanism in humans, and is not approved by the FDA. Lipofection entails administration of extraneous compounds to the patient in addition to the therapeutic agent itself. It is also not approved by the FDA.

Accordingly, new methods of efficient delivery of RNA to the patients are needed.

### SUMMARY OF THE INVENTION

The instant invention addresses these and other needs of the prior art by providing, in one aspect, a method of delivering RNA to a cell of a patient, comprising electrically stimulating an area in a patient's body containing said cell, with a low-voltage electric stimulation followed by delivery of the RNA. Preferably, the cell is selected from cardiomyocytes, skeletal muscle cells, kidney cells, neurons, and glial cells.

In other words, an aspect of the invention is related to a use of RNA in the manufacture of a medicament for use in a method of delivering said RNA to a cell of a patient, wherein a plurality of pulses of an electric field is or is to be applied to the cell for a time period between about two and about 24 hours, said RNA is or is to be administered after the administration of the plurality of pulses, and wherein the electric field has strength of between about 0.5 V/cm and about 40 V/cm, calculated according to Formula I:

E=V/d

wherein in said formula E is the strength, V is Voltage and d is distance between electrodes. Preferably, the cell is selected from cardiomyocytes, skeletal muscle cells, kidney cells, neurons, and glial cells.

The parameters of the electric stimulation are tailored to each cell type as to ensure the maximal efficiency of the RNA delivery.

In different embodiments, the RNA comprises a siRNA, an shRNA, an aptamer, a spiegelmer, and antimir, a template for a protein or a protein fragment, or a combination thereof.

In another aspect, the invention also provides a device suitable for the methods of the instant invention, the device generally comprising a plurality of electrodes and a catheter for delivery of the RNA.

In one embodiment, the device comprises a plurality of electrodes, a catheter, the catheter comprising a wall and a cavity, wherein the members of said plurality of electrodes are disposed within or on the surface of said wall, a reservoir containing a composition comprising the nucleic acid sequence, said reservoir fluidly connected with said catheter, a pump or syringe operably connected to said reservoir, a processor operably connected to the members of said plurality of electrodes and adapted to receive electrical signals from said members and to deliver an electric field to said members.

Preferably, the device of the instant invention is treated to ensure the absence of RNAse to prolong the lifespan of the administered RNA.

In another aspect, the invention provides a kit comprising a plurality of electrodes, a composition comprising RNA, a processor adapted to actuate an electric stimulation by the members of said plurality of electrodes receive a signal from the members of said plurality of electrodes and, within a predetermined time period after receiving said signal from the members of said plurality of electrodes to actuate release of at least a portion of said composition comprising RNA.

In another aspect, the invention provides a ribonucleic acid molecule for use in treating or preventing a disease, disorder or infection, wherein said ribonucleic acid molecule is for the administration to a cell within 24 hours of subjecting said cell to a plurality of pulses of an electric field for a time period between about two and about 24 hours, wherein the electric field has strength of between about 0.5 V/cm and about 40 V/cm, calculated according to Formula E=V/d wherein in said formula E is the strength, V is Voltage and d is distance between electrodes.

According to certain embodiments, the ribonucleic acid molecule is for inhibition of mRNA of a gene selected from the group consisting of DMPK, Antisense to KLHL1, JPH3, AR, IT15, DRPLA, SCA1, SCA2, SCA3/MJD, CACNA1A, SCA7, TBP, PABPN1, PHOX2B, ARX, HOXD13, α-synuclein, SOD1, TNF-a, IL-1b, SCN9A, IKBKB, RELA, IKBKG, PLN, BIM, RUNX2(runt-related transcription factor2, involved in in cleidocranial dysplasia) ZIC2 (Zic family member 2 involved in holoprosencephaly), HOXA13 (homeobox A13 involved in hand-foot-genital syndrome), FOXL2 (forkhead box L2 involved in type II blepharophimosis, ptosis, and epicanthus inversus syndrome), COMP (cartilage oligomeric matrix protein involved in multiple epiphyseal dysplasia.

In some embodiments, the cell may be selected from the group consisting of cardiac cells, skeletal muscle cells, kidney cells, neurons and glial cells.

In certain embodiments, the RNA of the instant invention is for the treatment of neurodegenerative diseases, kidney diseases, heart diseases, inflammation, and muscle disease.

In different embodiments of this aspect of the invention, the parameters of the electric field are tailored to each cell type as to ensure the maximal efficiency of the RNA delivery, as discussed in the description of the methods of the instant invention.

In different embodiments, RNA comprises a siRNA, an shRNA, an aptamer, a spiegelmer, and antimir, a template for a protein or a protein fragment, or a combination thereof

### BRIEF DESCRIPTION OF THE DRAWINGS

FIG. 1 demonstrates that the electric stimulation according to the instant invention does not affect cell viability.

FIG. 2 demonstrates that stimulation-mediated uptake of siRNA is frequency-dependent.

FIG. 3 demonstrates that the RNA uptake caused by electrical stimulation is due to a cell-based process.

FIG. 4 demonstrates that the RNA uptake caused by electrical stimulation is at least partially due to a caveolae-mediated endocytotic pathway.

FIG. 5 demonstrates different embodiments of the instant methods and the results thereof in different cell types.

FIG. 6 demonstrates the long-term effect of electrical stimulation on the RNA intake.

FIG. 7 demonstrates that RNA delivered according to the methods of the instant invention keeps its biologic function.

FIG. 8 is a schematic illustration of a device according to one embodiment of the instant invention.

FIG. 9 is a photographic image of a rat with an implanted device without a cap (FIG. 9A) and with the cap (FIG. 9B).

FIGS. 10A and 10B illustrate two protocols used for investigation of the RNA uptake in vivo.

FIG. 11 demonstrates that electrical stimulation improves local uptake of dsRNA in neuronal cells in vivo.

FIG. 12 demonstrates that neurons and glial cells are susceptible to pre-treatment with electric stimulation for enabling dsRNA uptake.

FIG. 13 demonstrates siRNA uptake in neurons within the dorsal and ventral gray horns of the spinal cord.

### DETAILED DESCRIPTION OF THE INVENTION

For purposes of better understanding the instant disclosure, the following non-limiting definitions have been provided:

The term “chronically implanted” with respect to a device refers to a device that remains in the body of a patient, after being positioned in a bodily tissue of the patient by a practitioner, for any period of time after the patient encounter with the practitioner is completed and the patient has departed from the presence of the practitioner.

The term “treating” or “treatment” of a disease refers to executing a protocol, which may include administering one or more drugs to a patient (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. In addition, “treating” or “treatment” does not require complete alleviation of signs or symptoms, does not require a cure, and specifically includes protocols which have only a marginal effect on the patient.

The term “patient” refers to a biological system to which a treatment can be administered. A biological system can include, for example, an individual cell, a set of cells (e.g., a cell culture), a pathogen, an organ, a tissue, or a multi-cellular organism. A “patient” can refer to a human patient or a non-human patient.

The term “practitioner” refers to a person who uses methods, kits and compositions of the current invention on the patient. The term includes, without limitations, doctors, nurses, scientists, and other medical or scientific personnel.

The terms “field strength” and “electric field strength” refer to a parameter equal to the ratio of voltage to distance between electrodes, i.e., E=V/d, wherein “E” is the field strength, “V” is the voltage and “d” is the distance between electrodes.

The term “uniform” as applied to a parameter of the electric field stimulation does not indicate absolute uniformity and may be 10% of the base parameter (e.g., field strength, duration, frequency, etc).

The term “about” will be understood by persons of ordinary skill in the art and will vary to some extent according to the context in which it is used. If there are uses of the term which are not clear to persons of ordinary skill in the art given the context in which it is used, “about” will mean up to plus or minus 10% of the particular term.

The terms “RNA” or “RNA agent” refer to nucleic acid molecules comprising a plurality of nucleotides. The majority of these nucleotides comprise ribose, rather than other sugars (e.g., deoxyribose). Thus, in one embodiment, the term RNA may refer to DNA-RNA hybrid, with a proviso that greater than 50% of the nucleotides comprise ribose. Preferably, such molecules comprises at least 55%, or at least 60%, or at least 65%, or at least 70%, or at least 75%, or at least 80%, or at least 85%, or at least 90%, or at least 95%, or at least 99% or 100% of nucleotides having ribose as a sugar.

The nucleotides may be modified in other ways, such as, for example, Such modifications include phosphorothioate linkages, fluorine-derivatized nucleotides (e.g., 2′-O-trifluoromethyl nucleotides2′-O-ethyl-trifluoromethoxy nucleotides, or 2′-O-difluoromethoxy-ethoxy nucleotides), deoxynucleotide overhangs, 2′-O-methylation, 2′-O-allylation, and locked nucleic acid (LNA) substitutions (Dorset and Tuschl, Nat. Rev. Drug Discov. 3:318 (2004); Gilmore et al., J. Drug Targeting 12:315 (2004)). Also see U.S. Patent Publication No. 20060270623 (McSwiggen).

In one embodiment, the 2′-deoxy-2′-fluoronucleotides are present at specifically selected locations in the RNAi agent that are sensitive to cleavage by ribonucleases, such as locations having pyrimidine nucleotides.

In a further set of embodiments, the RNAi agent may be chemically modified on a 3′ end, a 5′ end, or both the 3′ end and the 5′ end. These terminal modifications protect the nucleic acid molecule from exonuclease degradation and may help in delivery and/or localization within a cell. Examples of moieties suitable for the modification of the 5′ end of the RNAi agent include, without limitations, glyceryl, inverted deoxy abasic residue (moiety); 4′,5′-methylene nucleotide; 1-(beta-D-erythrofuranosyl)nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Non-limiting examples of the moieties suitable for modification of the 3′-end of the RNAi agent include glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties. Yet additional suitable modifications of the RNAi agent are described in details in U.S. patent application Ser. No. 11/450,856, filed on Jun. 9, 2006 (McSwiggen), which is incorporated herein by reference to the extent it is not inconsistent with the instant disclosure.

The RNA of the instant invention may be in a single-stranded form (i.e., ssRNA) or a double-stranded form (i.e., dsRNA).

The methods of the present invention utilize routine techniques in the field of molecular biology. Basic texts disclosing general molecular biology methods include Sambrook et al., Molecular Cloning, A Laboratory Manual (3d ed. 2001) and Ausubel et al., Current Protocols in Molecular Biology (1994).

In a general aspect, the instant invention provides methods for improved efficiency of delivering ribonucleic acids to cells and devices suitable for implementation of these methods.

The methods generally comprise stimulation of cells. In one aspect, the method comprises electrically stimulating an area in a patient's body containing said cell, with a low-voltage electric stimulation followed by delivery of the RNA.

In other words, an aspect of the invention is related to a use of RNA in the manufacture of a medicament for use in a method of delivering said RNA to a cell of a patient, wherein, in said method, a low-voltage electric stimulation is or is to be applied to the cell and said RNA is or is to be administered after the application of the low-voltage stimulation.

In different embodiments, the cells are selected from Adipocytes, Alzheimer type II astrocytes, Ameloblasts, Astrocytes, B cells, Basophil activation cells, Basophil granulocytes, Boettcher cells, Cementoblasts, Chondrocytes, Chromaffin cells, Clara cells, Corticotropes, Cytotoxic T cells, Dendritic cells, Eosinophil granulocytes, Extraglomerular mesangial cells, Gastric chief cells, Goblet cells, Gonadotropes, Hepatocytes, Hypersegmented neutrophils, Intraglomerular mesangial cells, Juxtaglomerular cells, Keratinocytes, Kidney proximal tubule brush border cells, Kupffer cells, Lactotrophs, Leydig cells, Macrophages, Macula densa cells, Magnocellulars, neurosecretory cells, Mast cells, Megakaryocytes, Melanocytes, Microglia, Monocytes, Myocardiocytes, Myocytes, Naive B cells, Natural Killer T cells, Natural killer cells, Neutrophil granulocytes, Nuclear chain cells, Osteoblasts, Osteoclasts, Osteocytes, Ovums, Oxyphil cells (parathyroid), Paneth cells, Parafollicular cells, Parathyroid chief cells, Parietal cells, Pericytes, Perivitelline space cells, Platelets, Pneumocytes, Podocytes, Pre-B cells, Red blood cells, Regulatory T cells, Reticulocytes, S cells, Sertoli cells, Somatotropes, Spermatozoons, Stellate cells, T helper cells, Tendon cells, Thrombocytes, Thyroid epithelial cells, Thyrotropes, Trichocytes (human), Type I pneumocytes, Type II pneumocytes, etc. Notably, the inventors were able to show unexpectedly advantageous results in neurons and muscle cells, which are known to be difficult to transfect. Thus, the other cell types disclosed above would also be susceptible to the methods described in the instant invention.

In other embodiments of invention, cells comprising pathogenic organisms (e.g., viruses) are treated.

In yet other embodiments, the treatment is directed against pathogenic organisms, including, without limitation, bacteria, fungi, protozoa, helmints, etc.

In certain embodiments, the cells are selected from the group consisting of brain cells (such as neurons and glial cells), cardiomyocytes, skeletal muscle cells, and kidney cells, with an electric field having specific parameters for a time period up to about 24 hours, prior to administering the RNA. Importantly, the parameters of the electric field strength are generally insufficient to perforate the membrane, and thus, the methods of the instant invention cannot be considered an “electroporation” in an art-accepted definition of that term (i.e., delivery of genetic material to cells via electrically caused perforations of respective cell membranes).

Generally, the electric stimulation of the instant invention comprises an electric field that has a strength of between about 0.5 V/cm and about 40 V/cm, calculated according to formula I:

E=V/d

wherein in said formula E is the strength, V is Voltage and d is distance between electrodes. The frequency of the stimulation is below about 400 Hz and the duration of each pulse is preferably between about 100 μs and about 500 μs.

In different embodiments, the strength of the electric field is between about 0.5 V/cm and about 2 V/cm, or between about 2 V/cm and about 6 V/cm, or between about 6 V/cm and about 12 V/cm, or between about 12 V/cm and about 28 V/cm, or between about 28 V/cm and about 40 V/cm. Preferably, the strength of the electric field is below 20 V/cm.

The pulse duration is between about 100 μs and about 200 μs, or between about 200 μs and about 300 μs, or between about 300μs and about 400 μs, or between about 400μs and about 500 μs.

In different embodiments, the frequency is between about 4 Hz and about 100 Hz, or between about 100 Hz and about 200 Hz or between about 200 Hz and about 250 Hz or between about 250 Hz and about 300 Hz, or between about 300 Hz and about 350 Hz, or between about 350 Hz and about 400 Hz.

Thus, the instant invention discloses 120 different treatment combinations. Ninety six of these combinations are summarized in the following table.

TABLE 1

| | Voltage range | Frequency range | Pulse duration |
|---|---|---|---|
| No. | (≈, V/cm) | (≈, Hz) | (≈, ms) |
| 1 | 0.5-2 | 100-200 | 4-100 |
| 2 | 0.5-2 | 100-200 | 100-200 |
| 3 | 0.5-2 | 100-200 | 200-250 |
| 4 | 0.5-2 | 100-200 | 250-300 |
| 5 | 0.5-2 | 100-200 | 300-350 |
| 6 | 0.5-2 | 100-200 | 350-400 |
| 7 | 0.5-2 | 200-300 | 4-100 |
| 8 | 0.5-2 | 200-300 | 100-200 |
| 9 | 0.5-2 | 200-300 | 200-250 |
| 10 | 0.5-2 | 200-300 | 250-300 |
| 11 | 0.5-2 | 200-300 | 300-350 |
| 12 | 0.5-2 | 200-300 | 350-400 |
| 13 | 0.5-2 | 300-400 | 4-100 |
| 14 | 0.5-2 | 300-400 | 100-200 |
| 15 | 0.5-2 | 300-400 | 200-250 |
| 16 | 0.5-2 | 300-400 | 250-300 |
| 17 | 0.5-2 | 300-400 | 300-350 |
| 18 | 0.5-2 | 300-400 | 350-400 |
| 19 | 0.5-2 | 400-500 | 0.5-2 |
| 20 | 0.5-2 | 400-500 | 0.5-2 |
| 21 | 0.5-2 | 400-500 | 0.5-2 |
| 22 | 0.5-2 | 400-500 | 0.5-2 |
| 23 | 0.5-2 | 400-500 | 0.5-2 |
| 24 | 0.5-2 | 400-500 | 0.5-2 |
| 25 | 2-6 | 100-200 | 4-100 |
| 26 | 2-6 | 100-200 | 100-200 |
| 27 | 2-6 | 100-200 | 200-250 |
| 28 | 2-6 | 100-200 | 250-300 |
| 29 | 2-6 | 100-200 | 300-350 |
| 30 | 2-6 | 100-200 | 350-400 |
| 31 | 2-6 | 200-300 | 4-100 |
| 32 | 2-6 | 200-300 | 100-200 |
| 33 | 2-6 | 200-300 | 200-250 |
| 34 | 2-6 | 200-300 | 250-300 |
| 35 | 2-6 | 200-300 | 300-350 |
| 36 | 2-6 | 200-300 | 350-400 |
| 37 | 2-6 | 300-400 | 4-100 |
| 38 | 2-6 | 300-400 | 100-200 |
| 39 | 2-6 | 300-400 | 200-250 |
| 40 | 2-6 | 300-400 | 250-300 |
| 41 | 2-6 | 300-400 | 300-350 |
| 42 | 2-6 | 300-400 | 350-400 |
| 43 | 2-6 | 400-500 | 4-100 |
| 44 | 2-6 | 400-500 | 100-200 |
| 45 | 2-6 | 400-500 | 200-250 |
| 46 | 2-6 | 400-500 | 250-300 |
| 47 | 2-6 | 400-500 | 300-350 |
| 48 | 2-6 | 400-500 | 350-400 |
| 49 | 6-12 | 100-200 | 4-100 |
| 50 | 6-12 | 100-200 | 100-200 |
| 51 | 6-12 | 100-200 | 200-250 |
| 52 | 6-12 | 100-200 | 250-300 |
| 53 | 6-12 | 100-200 | 300-350 |
| 54 | 6-12 | 100-200 | 350-400 |
| 55 | 6-12 | 200-300 | 4-100 |
| 56 | 6-12 | 200-300 | 100-200 |
| 57 | 6-12 | 200-300 | 200-250 |
| 58 | 6-12 | 200-300 | 250-300 |
| 59 | 6-12 | 200-300 | 300-350 |
| 60 | 6-12 | 200-300 | 350-400 |
| 61 | 6-12 | 300-400 | 4-100 |
| 62 | 6-12 | 300-400 | 100-200 |
| 63 | 6-12 | 300-400 | 200-250 |
| 64 | 6-12 | 300-400 | 250-300 |
| 65 | 6-12 | 300-400 | 300-350 |
| 66 | 6-12 | 300-400 | 350-400 |
| 67 | 6-12 | 400-500 | 4-100 |
| 68 | 6-12 | 400-500 | 100-200 |
| 69 | 6-12 | 400-500 | 200-250 |
| 70 | 6-12 | 400-500 | 250-300 |
| 71 | 6-12 | 400-500 | 300-350 |
| 72 | 6-12 | 400-500 | 350-400 |
| 73 | 12-20 | 100-200 | 4-100 |
| 74 | 12-20 | 100-200 | 100-200 |
| 75 | 12-20 | 100-200 | 200-250 |
| 76 | 12-20 | 100-200 | 250-300 |
| 77 | 12-20 | 100-200 | 300-350 |
| 78 | 12-20 | 100-200 | 350-400 |
| 79 | 12-20 | 200-300 | 4-100 |
| 80 | 12-20 | 200-300 | 100-200 |
| 81 | 12-20 | 200-300 | 200-250 |
| 82 | 12-20 | 200-300 | 250-300 |
| 83 | 12-20 | 200-300 | 300-350 |
| 84 | 12-20 | 200-300 | 350-400 |
| 85 | 12-20 | 300-400 | 4-100 |
| 86 | 12-20 | 300-400 | 100-200 |
| 87 | 12-20 | 300-400 | 200-250 |
| 88 | 12-20 | 300-400 | 250-300 |
| 89 | 12-20 | 300-400 | 300-350 |
| 90 | 12-20 | 300-400 | 350-400 |
| 91 | 12-20 | 400-500 | 4-100 |
| 92 | 12-20 | 400-500 | 100-200 |
| 93 | 12-20 | 400-500 | 200-250 |
| 94 | 12-20 | 400-500 | 250-300 |
| 95 | 12-20 | 400-500 | 300-350 |
| 96 | 12-20 | 400-500 | 350-400 |

It should be further understood that the methods and/or uses of the instant invention are sufficiently flexible as to allow embodiments where the parameters of the pulses are not uniform. Thus, for example, the pulses may vary in duration or amplitude, the bursts of pulses may vary in the interburst and intraburst characteristics. Such non-uniform electrical stimulation may be potentially advantageous for the embodiments wherein the electrical stimulation is administered to an organ with natural rhythmic electrical activity, such as, for example, heart.

Optionally, the RNA of the instant invention may be administered to the cells within a composition comprising an imaging agent. This embodiment will allow verification of distribution of the composition comprising the RNA, thus optimizing the targeting of the desired cells within the patient's body.

Using the methods of the instant invention, including, without limitation, the combinations selected from those disclosed in Table 1, it is possible to increase the uptake of the RNA by up to at least about 1000% (e.g., at least by about 25%, about 50%, about 75%, about 100%, about 125%, about 150%, about 175%, about 200%, about 250%, about 300%, about 350%, about 400%, about 450%, about 500%, about 550%, about 600%, about 650%, about 700%, about 750%, about 800%, about 850%, about 900%, about 950%).

Stimulation of Brain Cells

The parameters of the stimulation with the electric field are specifically tailored to the targeted cell types. If the selected cells are brain cells, e.g., neurons, advantageously, the parameters for the electric stimulation may be those used for DBS, or deep brain stimulation. For example, the inventors have surprisingly discovered that the field strength of about 28 V/cm administered for about 2 hours results in increased uptake of the RNA by Neuro2a cells.

Thus, in different embodiments of the method of administration of RNA to neuronal cells, the following parameters may be used:

a) field strength of greater than about 0.5, 1, 2, 3, 4, 5, 6, 7, 8, 9, 10, 11, 12, 13, 14, 15, 16, 17, 18, 19, 20, 21, 22, 23, 24, 25, 26, or 27 and up to about 28 V/cm;

b) duration of treatment of at least about 2, 4, 6, 8, 10, 12, 14, 16, 18, 20, 22 and up to about 24 hours;

c) frequency of greater than about 20 Hz, 40 Hz, 60 Hz, 80 Hz, 100 Hz, 120 Hz, 140 Hz, 160 Hz, 180 Hz, or 200 Hz;

d) pulse width of at least about 0.1 msec, 0.2 msec, 0.3 msec, 0.4 msec, 0.5 msec, 0.6 msec, 0.7 msec, 0.8 msec, 0.9 msec, or 1 msec. In some embodiments, the pulses of the electric stimulation may be administered in bursts, such as, for example bursts of several pulses separated by a time interval. The time interval may be greater than about 100 msec, 200 msec, 300 msec, 400 msec, 500 msec, 600 msec, 700 msec, 800 msec, 900 msec, and 1 sec.

In one selected embodiment, the electric field strength is between about 2 and about 12 V/cm, the duration of electric stimulation is greater than about 1 and less than 24 hours (e.g., about 2, 3, 4, 5, 6, 7, 8, 9, 10, 11, 12, 13, 14, 15, 16, 17, 18, 19, 20, 21, 22, 23 hrs), pulse duration is between about 0.1 msec and about 0.5 msec (e.g., about 0.3 msec), and the frequency of the pulses is between about 50 Hz and about 150 Hz (e.g., about 100 Hz).

In another embodiment, the electric field strength is greater than 24 and less than 40 V/cm (e.g., between 26 and 30 V/cm, preferably, about 28 V/cm), with the duration of the stimulation of less than six hours (e.g., about 5, or about 4, or about 3, or about 2, or about 1, or about 0.5 hr), pulse frequency of between about 50 Hz and about 150 Hz (e.g., about 100 Hz) and pulse duration between about 0.1 msec and about 0.5 msec (e.g., about 0.3 msec).

The inventors have also discovered that even though high field strength (e.g., about 28 V/cm) successfully improves uptake of RNA into neuronal cells, some cytotoxicity may be seen in vitro and in vivo. Accordingly, in yet another embodiment, low field strength (below 20 V/cm, such as, for example 10, 6, or 2 V/cm) may be used, for as little as two hours (but, of course, the duration may be greater than two hours), with pulse frequency of between about 50 Hz and about 150 Hz (e.g., about 100 Hz) and pulse duration between about 0.1 msec and about 0.5 msec (e.g., about 0.3 msec).

Notably, Deep Brain Stimulation (DBS) for patients with certain diseases (e.g., Parkinson's disease or Essential tremor) fall within these ranges. For example, typical stimulus parameters employed for DBS for movement disorders are in the range of 2-4 V (or 2-4 mA for a typical DBS electrode impedance of 1,000), 90-180 μs pulse width, and 100-185 Hz. Testerman RL et al., IEEE Eng Med Biol Mag. 2006 September-October; 25(5):74-8. Review. Thus, in selected embodiments, the methods of the instant invention may be administered in conjunction with the DBS to a patient in need thereof, wherein the DBS provides the electrical stimulation according to the instant invention.

Using the parameters outlined in this section, one may improve the uptake of RNA by up to at least about 1000% (e.g., at least by about 25%, about 50%, about 75%, about 100%, about 125%, about 150%, about 175%, about 200%, about 250%, about 300%, about 350%, about 400%, about 450%, about 500%, about 550%, about 600%, about 650%, about 700%, about 750%, about 800%, about 850%, about 900%, about 950%).

Inventors have also discovered that out of the multiple cell types present in the brain, neurons are among the cell types most susceptible to the electric field stimulation. Thus, in another embodiment of the invention, the electric stimulation (and the RNA therapy) may also be used for treatment of any part of the patient's body where neuronal bodies are present and where uptake of dsRNA into neurons is desired. For example, such body parts may include spinal cord, dorsal root ganglia, brain and neurons comprising the peripheral nervous system, and enteric nervous system.

Stimulation of Cardiac and Skeletal Muscle Cells.

In this set of embodiments of the instant invention, the electric field is preferably less than 28 V/cm, preferably less than 20 V/cm, more preferably, between about 0.5 V/cm and about 12 V/cm, even more preferably, between about 2 V/cm and about 12 V/cm, e.g., 3, 4, 5, 6, 7, 8, 9, 10, or 11 V/cm.

Due to natural electric activity of the heart muscle, it may be advantageous to tailor the electric stimulation parameters to the natural rhythms of the heart. Thus, especially advantageous embodiments are those where the pulses are administered in bursts. It is necessary to note, nevertheless, that the stimulation in burst is not absolutely necessary to successfully improve RNA uptake by the targeted cells. Thus, in different embodiments, the frequency of stimulation is between about 10 Hz and about 150 Hz. In selected embodiments, the frequency may be between about 80 Hz and about 120 Hz, e.g., about 90 Hz, about 100 Hz, or about 110 Hz. In other embodiments, the stimulation is delivered in bursts, as discussed above. For example, the frequency may be between about 10 Hz and about 50 Hz, e.g., about 15 Hz, about 20 Hz, about 25 Hz, about 30 Hz, about 35 Hz, about 40 Hz, about 45 Hz). In an exemplary embodiment, the frequency is about 20 Hz, and the stimulation comprises bursts of 10 pulses, separated by 500 msec, with the duration of each pulse of about 0.3 msec. However, these numbers (i.e., duration of each pulse, number of bursts, number of pulses in each burst, and interburst interval) may be varied (e.g., the duration of each pulse of about 0.5 msec and correspondingly decreased interburst interval).

The duration of the electric field treatment may be varied between about two and about 24 hours, e.g., 4 hrs, 6, hrs, 8 hrs, 10 hrs, 12 hrs, 14 hrs, 16 hrs, 18 hrs, 20 hrs, 22 hrs.

In selected embodiments, the method comprises determining the heart rate of a patient, and then administering the electrical stimulation according to that heart rate. Further, the bursts of the heart activity may be sensed, and the occurrence of these bursts would provide a signal for administering the respective bursts of the electrical stimulation.

Using these parameters, one can improve RNA intake by up to at least about 1000% (e.g., at least by about 25%, about 50%, about 75%, about 100%, about 125%, about 150%, about 175%, about 200%, about 250%, about 300%, about 350%, about 400%, about 450%, about 500%, about 550%, about 600%, about 650%, about 700%, about 750%, about 800%, about 850%, about 900%, about 950%).

Stimulation of Kidney Cells

In another embodiment of the invention, kidney cells are treated with the combination of electric field stimulation and RNA. The inventors have surprisingly discovered that kidney cells are susceptible to the wide varieties of the electric field stimulation. Thus, if the kidney cells are to be treated according to the methods of the instant invention, the strength of the electric field may be in a range of between about 0.5 V/cm and about 60 V/cm, such as for example, 0.8 V/cm, 1 V/cm, 2 V/cm, 5 V/cm, 10 V/cm, 12 V/cm, 15 V/cm, 20 V/cm, 25 V/cm, 20 V/cm, 25 V/cm, 30 V/cm, and 35 V/cm. The frequency of the stimulation may be as low as e.g., about 4 Hz and as high as at least 100 Hz. The duration of the stimulus may be in the range between about 0.1 msec and about 1 msec, e.g., 0.2 msec, 0.3 msec, 0.4 msec, 0.5 msec, 0.6 msec, 0.7 msec, 0.8 msec, or 0.9 msec. The precise duration of the electric stimulation is also not crucial and may be from about 1 hr to about 24 hours, e.g., 2, 3, 4, 5, 6, 7, 8, 9, 10, 11, 12, 13, 14, 15, 16, 17, 18, 19, 20, 21, 22 or 23 hours.

Using these parameters, one can improve RNA intake by up to at least about 1000% (e.g., at least by about 25%, about 50%, about 75%, about 100%, about 125%, about 150%, about 175%, about 200%, about 250%, about 300%, about 350%, about 400%, about 450%, about 500%, about 550%, about 600%, about 650%, about 700%, about 750%, about 800%, about 850%, about 900%, about 950%).

RNA

It is important to note that for all embodiments, the RNA may, but does not need to, be administered immediately after the electric field stimulation. In various embodiments, the RNA is administered up to about 24 hours after the disclosed cells have been stimulated for the predetermined amount of time, as described above (e.g., 0.5, 1, 2, 3, 4, 5, 5, 6, 7, 8, 9, 10, 11, 12, 13, 14, 15, 16, 17, 18, 19, 20, 21, 22, 23 hours). It is also important to know that the electric field stimulation does not need to stop when RNA is administered. Thus, in some embodiments, the cells may be pre-treated with electric stimulation for the desired amount of time, and then essentially co-treated with the electric stimulation and the RNA. Thus, in additional embodiments, the RNA is delivered to the cells at least 1 minute, at least 5 minutes, at least 30 minutes, at least 45 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least hours, at least 21 hours, at least 22 hours, at least 23 hours or 24 hours after the cells have been stimulated. However, as demonstrated in the examples, it is important not to withdraw the RNA treatment within a short time after the electrical stimulation.

Thus, generally, the practitioner of the described method may stimulate the disclosed cells according to the suitable parameters as recited above, then optionally wait for a desired amount of time (up to about 24 hours, as noted in the previous paragraph), and then administer the RNA according to this invention. During the optional waiting time (or any part thereof) or during the RNA delivery, the electric field stimulation may be on or off.

The RNA agents of the instant invention may be used for multiple purposes, such as, for example, as RNA interference agents or to supplement expression of a desired gene. Most generally, the RNA agents may be used either to suppress expression of an undesirable gene (e.g., huntingtin, SCA-1, etc) or to suppress gene expression and/or function implicated in the pathogenesis of a disorder (BACE-1, alpha synuclein, ect). Table 2 describes in part examples of triplet repeat expansion diseases and the mutant gene associated with each disease.

TABLE 2
Triplet Repeat Expansion Disorders
Non-coding repeats

| Disease | Symptoms | Gene | Locus | Protein |
|---|---|---|---|---|
| Dystrophia | Weakness, | DMPK | 19q13 | Dystrophia |
| myotonica 1 | Myotonia | | | myotonica |
| | | | | Protein kinase |
| Spinocerebellar | Ataxia | Antisense | 13q21 | Undetermined |
| ataxia 8 | | to KLHL1 | | |
| Huntington disease- | Chorea, dementia | JPH3 | 16q24.3 | Junctophilin 3 |
| like2 | | | | |

Polyglutamine disorders

| | | | | |
|---|---|---|---|---|
| Spinal and bulbar | Weakness | AR | Xq13-q21 | Androgen |
| muscular atrophy | | | | receptor |
| Huntington disease | Chorea, dementia | IT15 | 4P16.3 | Huntingtin |
| Dentatorubral- | Ataxia, myoclonic | DRPLA | 12p13.31 | Atrophin 1 |
| pallidoluysian | epilepsy, | | | |
| atrophy | dementia | | | |
| Spinocerebellar | Ataxia | SCA1 | 6p23 | Ataxin 1 |
| ataxia 1 | | | | |
| Spinocerebellar | Ataxia | SCA2 | 12q24.1 | Ataxin 2 |
| ataxia 2 | | | | |
| Spinocerebellar | Ataxia | SCA3/MJD | 14q32.1 | Ataxin 3 |
| ataxia 3 (Machado- | | | | |
| Joseph disease) | | | | |
| Spinocerebellar | Ataxia | CACNA1A | 19p13 | α_{1A}-voltage- |
| ataxia 6 | | | | dependent |
| | | | | calcium |
| | | | | channel |
| | | | | subunit |
| Spinocerebellar | Ataxia | SCA7 | 3p12-p13 | Ataxin 7 |
| ataxia 7 | | | | |
| Spinocerebellar | Ataxia | TBP | 6q27 | TATA box |
| ataxia 17 | | | | binding |
| | | | | protein |

Polyalanine disorders*

| | | | | |
|---|---|---|---|---|
| Oculopharyngeal | Weakenss | PABPN1 | 14q11.2-q13 | Poly(A)- |
| dystrophy | | | | binding |
| | | | | protein 2 |
| Congential central | Respiratory | PHOX2B | 4p12 | Paired-like |
| hypoventilation | difficulties | | | homeobox 2B |
| syndrome | | | | |
| Infantile spasms | Mental | ARX | Xp22.13 | Aristaless- |
| | retardation, | | | related |
| | epilepsy | | | homeobox, X- |
| | | | | linked |
| Synpolydactyly | Limb malformation | HOXD13 | 2q31-q32 | Homeobox D13 |

*Polyalanine expansions have also been reported among mutations in other genes, including RUNX2 (runt-related transcription factor2) in cleidocranial dysplasia, ZIC2 (Zic family member 2) in holoprosencephaly HOXA13 (homeobox A13) in hand-foot-genital syndrome, and FOXL2 (forkhead box L2) in type II blepharophimosis, ptosis, and epicanthus inversus syndrome. Small aspartic acid repeat expansions have been reported among other mutations in the COMP (cartilage oligomeric matrix protein)gene in patients with multiple epiphyseal dysplasia.

Suitable classes of RNA agents for RNA interference include, without limitations, siRNA, shRNA and miRNA. These agents may target selected regions within their respective target mRNAs, including, without limitations, sequences recited in the following Table 3.

TABLE 3

| | | | |
|---|---|---|---|
| | | | Target Gene |
| SEQ ID NO: | Target Gene | Nucleotide Base Sequence | Accession # |

| | | | |
|---|---|---|---|
| 1 | SCA1 | AACCAAGAGCGGAGCAACGAA | NM_000332.3 |
| 2 | IT15 (Htt) | TGACAGCAGTGTTGATAAA | NM_002111.6 |
| 3 | IT15 (Htt) | AAGAACGAGTGCTCAATAA | NM_002111.6 |
| 4 | IT15 (Htt) | TTTATGAACTGACGTTACA | NM_002111.6 |
| 5 | IT15 (Htt) | GGAGTATTGTGGAACTTAT | NM_002111.6 |
| 6 | IT15 (Htt) | GAGTATTGTGGAACTTATA | NM_002111.6 |
| 7 | IT15 (Htt) | AGACCGTGTGAATCATTGT | NM_002111.6 |
| 8 | IT15 (Htt) | GGTTACAGCTCGAGCTCTA | NM_002111.6 |
| 9 | IT15 (Htt) | GGTTTTGTTAAAGGCCTTC | NM_002111.6 |
| 10 | IT15 (Htt) | TGACAGCAGTGTTGATAAATTTGTGTT | NM_002111.6 |
| 11 | IT15 (Htt) | AAGAACGAGTGCTCAATAATGTTGTCA | NM_002111.6 |
| 12 | IT15 (Htt) | TTTATGAACTGACGTTACATCATACAC | NM_002111.6 |
| 13 | IT15 (Htt) | GGAGTATTGTGGAACTTATAGCTGGAG | NM_002111.6 |
| 14 | IT15 (Htt) | GAGTATTGTGGAACTTATAGCTGGAGG | NM_002111.6 |
| 15 | IT15 (Htt) | AGACCGTGTGAATCATTGTCTGACAAT | NM_002111.6 |
| 16 | IT15 (Htt) | GGTTTTGTTAAAGGCCTTCATAGCGAA | NM_002111.6 |
| 17 | BACE1 | AAGGGTGTGTATGTGCCCTAC | NM_012104.3 |
| 18 | BACE1 | AATTGGCTTTGCTGTCAGCGC | NM_012104.3 |
| 19 | BACE1 | AAGACTGTGGCTACAACATTC | NM_012104.3 |
| 20 | BACE1 | AAGGCTGCCTGGAGAAAGGAT | NM_012104.3 |
| 21 | BACE1 | CACTGAATCGGACAAGTTCTT | NM_012104.3 |
| 22 | BACE1 | CATGATCATTGGTGGTATCGA | NM_012104.3 |
| 23 | BACE1 | CATCCTTCCTCAGCAATACCT | NM_012104.3 |
| 24 | BACE1 | CAGACGCTCAACATCCTGGTG | NM_012104.3 |
| 25 | α-synuclein | CTACGAACCTGAAGCCTAA | NM_000345.3 |
| 26 | α-synuclein | TCAAGACTACGAACCTGAA | NM_000345.3 |
| 27 | α-synuclein | CATTAGCCATGGATGTATT | NM_000345.3 |
| 28 | α-synuclein | ACGAACCTGAAGCCTAAGA | NM_000345.3 |
| 29 | α-synuclein | GTACAAGTGCTCAGTTCCA | NM_000345.3 |
| 30 | α-synuclein | GCTTCAATCTACGATGTTA | NM_000345.3 |
| 31 | α-synuclein | CTAAGTGACTACCACTTAT | NM_000345.3 |
| 32 | α-synuclein | GTTCAGAAGTTGTTAGTGA | NM_000345.3 |
| 33 | α-synuclein | AGTTGTTAGTGATTTGCTA | NM_000345.3 |
| 34 | α-synuclein | GACGTATTGTGAAATTTGT | NM_000345.3 |
| 35 | SOD1 | TCATCAATTTCGAGCAGAA | NM_000454.4 |
| 36 | SOD1 | TGAGTTTGGAGATAATACA | NM_000454.4 |
| 37 | SOD1 | TGGCCGATGTGTCTATTGA | NM_000454.4 |
| 38 | SOD1 | CGATGTGTCTATTGAAGAT | NM_000454.4 |
| 39 | SOD1 | GCATTAAAGGACTGACTGA | NM_000454.4 |
| 40 | SOD1 | TCGTTTGGCTTGTGGTGTA | NM_000454.4 |
| 41 | SOD1 | AATTTCGAGCAGAAGGAAAGT | NM_000454.4 |
| 42 | SOD1 | AAGCATTAAAGGACTGACTGA | NM_000454.4 |
| 43 | SOD1 | AATGTGACTGCTGACAAAGAT | NM_000454.4 |
| 44 | SOD1 | AAGATTCTGTGATCTCACTCT | NM_000454.4 |
| 45 | TNF-a | CCAGGGACCTCTCTCTAAT | NM_000594.2 |
| 46 | TNF-a | AGGGACCTCTCTCTAATCA | NM_000594.2 |
| 47 | TNF-a | GCCTGTAGCCCATGTTGTA | NM_000594.2 |
| 48 | TNF-a | TGTAGCCCATGTTGTAGCA | NM_000594.2 |
| 49 | IL-1b | GTGAAATGATGGCTTATTA | NM_000576.2 |
| 50 | IL-1b | CGATGCACCTGTACGATCA | NM_000576.2 |
| 51 | IL-1b | TAACTGACTTCACCATGCA | NM_000576.2 |
| 52 | IL-1b | GAACCTATCTTCTTCGACA | NM_000576.2 |
| 53 | SCN9A | CAGAAGAACAGAAGAAATA | NM_002977.2 |
| 54 | SCN9A | TGAAGAAGCTAAACAGAAA | NM_002977.2 |
| 55 | SCN9A | GGTAAGAGCTACAAAGAAT | NM_002977.2 |
| 56 | SCN9A | AGGCAGAGGAAGAGATATA | NM_002977.2 |
| 57 | SCN9A | AGACAGAGATGATGATTTA | NM_002977.2 |
| 58 | SCN9A | GGGAAAGACAGCAAGGAAA | NM_002977.2 |
| 59 | SCN9A | GAACAAGACAGAACAGAAA | NM_002977.2 |
| 60 | SCN9A | GTGAAGAAGACTTTAGAAA | NM_002977.2 |
| 61 | SCN9A | CCAAAGATTTCCAGGGAGA | NM_002977.2 |
| 62 | SCN9A | TAACATAGAGTCAGGGAAA | NM_002977.2 |
| 63 | SCN9A | GAAAGAAGAAACAGAAGAA | NM_002977.2 |
| 64 | SCN9A | GGAGATAAGACAAGCAGAA | NM_002977.2 |
| 65 | SCN9A | CTGAATACTAAGAAGGAAA | NM_002977.2 |
| 66 | SCN9A | GAGAAGAAGCAGAGGCTGA | NM_002977.2 |
| 67 | SCN9A | GAAAGATGATGATGAAGAA | NM_002977.2 |
| 68 | SCN9A | TGGGAAACCTGAAGCATAA | NM_002977.2 |
| 69 | SCN9A | GAACACAGTTGGTTTGAAA | NM_002977.2 |
| 70 | SCN9A | TGACAGAAGAACAGAAGAA | NM_002977.2 |
| 71 | SCN9A | AAGAAGAAGCTGAGGCAAT | NM_002977.2 |
| 72 | SCN9A | TTTCAAAGGCAGAGGAAGA | NM_002977.2 |
| 73 | SCN9A | CTTGAAGAGTCCAGACAAA | NM_002977.2 |
| 74 | SCN9A | GCTAAAGAAAGAAGAAACA | NM_002977.2 |
| 75 | SCN9A | AGAAGAAACAGAAGAAAGA | NM_002977.2 |
| 76 | SCN9A | GCTGAGAAATTGTCGAAAT | NM_002977.2 |
| 77 | SCN9A | GAGCAAGCATATTAACAAA | NM_002977.2 |
| 78 | SCN9A | CATAAAAGATGGAGACAGA | NM_002977.2 |
| 79 | SCN9A | TAACAAAGCCAGACAAAGA | NM_002977.2 |
| 80 | SCN9A | AAAGGAAGACAAAGGGAAA | NM_002977.2 |
| 81 | SCN9A | AAAGGGAGATGCTGAGAAA | NM_002977.2 |
| 82 | SCN9A | TAACAAACACTGTGGAAGA | NM_002977.2 |
| 83 | SCN9A | AGTATTGAACAAAGGGAAA | NM_002977.2 |
| 84 | SCN9A | AGGCGAAGCAGCAGAACAA | NM_002977.2 |
| 85 | SCN9A | TAGCAGATGTGGAAGGATT | NM_002977.2 |
| 86 | SCN9A | AAACAAACCTTACGTGAAT | NM_002977.2 |
| 87 | SCN9A | AAATATGAATGCTGAGGAA | NM_002977.2 |
| 88 | SCN9A | CCAAAGAAGAAAAGAAAGA | NM_002977.2 |
| 89 | SCN9A | CTGACAAACTGCATATTTA | NM_002977.2 |
| 90 | SCN9A | AGGGAGATGCTGAGAAATT | NM_002977.2 |
| 91 | SCN9A | CATTGAACATGCTGATTAA | NM_002977.2 |
| 92 | SCN9A | GCATGCAGCTCTTTGGTAA | NM_002977.2 |
| 93 | SCN9A | AGACAATCTTACAGCAATT | NM_002977.2 |
| 94 | SCN9A | AAGAAGACCCTGATGCAAA | NM_002977.2 |
| 95 | SCN9A | GGAAGACAGTGATGGTCAA | NM_002977.2 |
| 96 | SCN9A | CAGACAAGATCTTCACTTA | NM_002977.2 |
| 97 | SCN9A | AGCCAGACAAAGAGAAATA | NM_002977.2 |
| 98 | SCN9A | CTTCGAACTTTCAGAGTAT | NM_002977.2 |
| 99 | SCN9A | GAGTAGAGCAAGCATATTA | NM_002977.2 |
| 100 | SCN9A | TGTACTTGCTATAGGAAAT | NM_002977.2 |
| 101 | SCN9A | GGTCAAGCTATGTGCCTTA | NM_002977.2 |
| 102 | SCN9A | GAAACAAACCTTACGTGAA | NM_002977.2 |
| 103 | SCN9A | GATTATGGCTACACGAGCT | NM_002977.2 |
| 104 | SCN9A | GATGGATTCTCTTCGTTCA | NM_002977.2 |
| 105 | SCN9A | TGTTTCAGCTCTTCGAACT | NM_002977.2 |
| 106 | IKBKB | CCGACATTGTGGACTTACA | NM_001556.1 |
| 107 | IKBKB | GCTTAGATACCTTCATGAA | NM_001556.1 |
| 108 | IKBKB | GGGAACAAGACCAGAGTTT | NM_001556.1 |
| 109 | IKBKB | AGATTGACCTGGAGAAGTA | NM_001556.1 |
| 110 | IKBKB | CTGCTGGCCTGGAGGGAAA | NM_001556.1 |
| 111 | IKBKB | GCTTAATGAATGAGGATGA | NM_001556.1 |
| 112 | IKBKB | CAGCAGAAGTACACAGTGA | NM_001556.1 |
| 113 | IKBKB | GGACATTGTTGTTAGCGAA | NM_001556.1 |
| 114 | IKBKB | ACTTAAAGCTGGTTCATAT | NM_001556.1 |
| 115 | IKBKB | TGACAGAGGATGAGAGTCT | NM_001556.1 |
| 116 | IKBKB | CTGCAGAGCTTGAAGGCCA | NM_001556.1 |
| 117 | IKBKB | GAGCTGTACAGGAGACTAA | NM_001556.1 |
| 118 | IKBKB | GAGAAGAAAGTGCGAGTGA | NM_001556.1 |
| 119 | IKBKB | GGAGAAGTACAGCGAGCAA | NM_001556.1 |
| 120 | IKBKB | AGAAAGTGCGAGTGATCTA | NM_001556.1 |
| 121 | IKBKB | GAAAGAGCGCCTTGGGACA | NM_001556.1 |
| 122 | IKBKB | GCTTCAAGGCCCTGGATGA | NM_001556.1 |
| 123 | IKBKB | GGTTACAGACGGAAGAAGA | NM_001556.1 |
| 124 | IKBKB | TGATGAATCTCCTCCGAAA | NM_001556.1 |
| 125 | IKBKB | AAGTGAAACTCCTGGTAGA | NM_001556.1 |
| 126 | IKBKB | GGAAACAGGTGAGCAGATT | NM_001556.1 |
| 127 | IKBKB | GCAAGTTAAATGAGGGCCA | NM_001556.1 |
| 128 | IKBKB | GAAGAAAGTGCGAGTGATC | NM_001556.1 |
| 129 | IKBKB | ATGAATGCCTCTCGACTTA | NM_001556.1 |
| 130 | IKBKB | GGGCCTGGGAAATGAAAGA | NM_001556.1 |
| 131 | IKBKB | GCGAAGACTTGAATGGAAC | NM_001556.1 |
| 132 | IKBKB | CCAATAATCTTAACAGTGT | NM_001556.1 |
| 133 | IKBKB | TTCAAGAGCCCAAGAGGAA | NM_001556.1 |
| 134 | IKBKB | GGGAAATGGAGCAGGCTGT | NM_001556.1 |
| 135 | IKBKB | AGACCGACATTGTGGACTT | NM_001556.1 |
| 136 | IKBKB | TACAGGAGACTAAGGGAAA | NM_001556.1 |
| 137 | IKBKB | GAAGAGGTGGTGAGCTTAA | NM_001556.1 |
| 138 | IKBKB | GGGAAATGAAAGAGCGCCT | NM_001556.1 |
| 139 | IKBKB | ACACAGTGACCGTCGACTA | NM_001556.1 |
| 140 | IKBKB | ATGAAGAATTCCATGGCTT | NM_001556.1 |
| 141 | IKBKB | GTTACAGACGGAAGAAGAA | NM_001556.1 |
| 142 | IKBKB | GGAAGTACCTGAACCAGTT | NM_001556.1 |
| 143 | IKBKB | TGGGATCACATCAGATAAA | NM_001556.1 |
| 144 | IKBKB | GAGCTTAATGAATGAGGAT | NM_001556.1 |
| 145 | IKBKB | CCAAGAAGAGTGAAGAACT | NM_001556.1 |
| 146 | IKBKB | TGACATTGCCTCTGCGCTT | NM_001556.1 |
| 147 | IKBKB | TGGCTGAGCGACTGGAGAA | NM_001556.1 |
| 148 | IKBKB | TTTCAGACGGCAAGTTAAA | NM_001556.1 |
| 149 | IKBKB | TGGACGACCTAGAGGAGCA | NM_001556.1 |
| 150 | IKBKB | TAATGAATGAGGATGAGAA | NM_001556.1 |
| 151 | IKBKB | AGCCAAGAAGAGTGAAGAA | NM_001556.1 |
| 152 | IKBKB | CAAGAAGAGTGAAGAACTG | NM_001556.1 |
| 153 | IKBKB | AGAAGAGTGAGGTGGACAT | NM_001556.1 |
| 154 | IKBKB | AGCGAAGACTTGAATGGAA | NM_001556.1 |
| 155 | IKBKB | AGGAAACAGGTGAGCAGAT | NM_001556.1 |
| 156 | IKBKB | CTTAGATACCTTCATGAAA | NM_001556.1 |
| 157 | RELA | GCATCCAGACCAACAACAA | NM_021975.3 |
| 158 | RELA | AGCGCATCCAGACCAACAA | NM_021975.3 |
| 159 | RELA | GTGACAAGGTGCAGAAAGA | NM_021975.3 |
| 160 | RELA | GGATTGAGGAGAAACGTAA | NM_021975.3 |
| 161 | RELA | CCCACGAGCTTGTAGGAAA | NM_021975.3 |
| 162 | RELA | GGAGAAACGTAAAAGGACA | NM_021975.3 |
| 163 | RELA | GGCGAGAGGAGCACAGATA | NM_021975.3 |
| 164 | RELA | CTACACAGGACCAGGGACA | NM_021975.3 |
| 165 | RELA | AAGAGGACATTGAGGTGTA | NM_021975.3 |
| 166 | RELA | GGAAAGGACTGCCGGGATG | NM_021975.3 |
| 167 | RELA | TCAAGAGCATCATGAAGAA | NM_021975.3 |
| 168 | RELA | TGGAGTACCCTGAGGCTAT | NM_021975.3 |
| 169 | RELA | GAATCCAGTGTGTGAAGAA | NM_021975.3 |
| 170 | RELA | TCAGTGAGCCCATGGAATT | NM_021975.3 |
| 171 | RELA | CGTAAAAGGACATATGAGA | NM_021975.3 |
| 172 | RELA | GGGAAGGAACGCTGTCAGA | NM_021975.3 |
| 173 | RELA | CCACGAGCTTGTAGGAAAG | NM_021975.3 |
| 174 | RELA | CTTCCAAGTTCCTATAGAA | NM_021975.3 |
| 175 | RELA | GCATCCAGACCAACAACAA | NM_021975.3 |
| 176 | RELA | CTCAAGATCTGCCGAGTGA | NM_021975.3 |
| 177 | RELA | CGGATTGAGGAGAAACGTA | NM_021975.3 |
| 178 | RELA | GATTGAGGAGAAACGTAAA | NM_021975.3 |
| 179 | RELA | GGACATATGAGACCTTCAA | NM_021975.3 |
| 180 | RELA | TCACCGGATTGAGGAGAAA | NM_021975.3 |
| 181 | RELA | CAACTGAGCCCATGCTGAT | NM_021975.3 |
| 182 | RELA | AGGAAAGGACTGCCGGGAT | NM_021975.3 |
| 183 | RELA | CCAACACTGCCGAGCTCAA | NM_021975.3 |
| 184 | RELA | GCTGCAGTTTGATGATGAA | NM_021975.3 |
| 185 | RELA | GACCAGGGACAGTGCGCAT | NM_021975.3 |
| 186 | RELA | GGGATGAGATCTTCCTACT | NM_021975.3 |
| 187 | RELA | AGGTGCAGAAAGAGGACAT | NM_021975.3 |
| 188 | RELA | AGGACATTGAGGTGTATTT | NM_021975.3 |
| 189 | RELA | GAGAAACGTAAAAGGACAT | NM_021975.3 |
| 190 | RELA | CATCAAGATCAATGGCTAC | NM_021975.3 |
| 191 | RELA | CCAAGTTCCTATAGAAGAG | NM_021975.3 |
| 192 | RELA | AGATCTTCCTACTGTGTGA | NM_021975.3 |
| 193 | RELA | AGAAAGAGGACATTGAGGT | NM_021975.3 |
| 194 | RELA | GTCACCGGATTGAGGAGAA | NM_021975.3 |
| 195 | RELA | ACATATGAGACCTTCAAGA | NM_021975.3 |
| 196 | RELA | GCTATAACTCGCCTAGTGA | NM_021975.3 |
| 197 | RELA | TGCAGAAAGAGGACATTGA | NM_021975.3 |
| 198 | RELA | GAAAGAGGACATTGAGGTG | NM_021975.3 |
| 199 | RELA | AAGCTGATGTGCACCGACA | NM_021975.3 |
| 200 | RELA | GATCAATGGCTACACAGGA | NM_021975.3 |
| 201 | RELA | TGTGTGACAAGGTGCAGAA | NM_021975.3 |
| 202 | RELA | TTCCAGTACCTGCCAGATA | NM_021975.3 |
| 203 | RELA | GAGGAGAAACGTAAAAGGA | NM_021975.3 |
| 204 | RELA | TATGAGACCTTCAAGAGCA | NM_021975.3 |
| 205 | RELA | ATGAGACCTTCAAGAGCAT | NM_021975.3 |
| 206 | RELA | AAGAGCATCATGAAGAAGA | NM_021975.3 |
| 207 | RELA | TGGCAACAGCACAGACCCA | NM_021975.3 |
| 208 | RELA | CAAGATCAATGGCTACACA | NM_021975.3 |
| 209 | IKBKG | GGAAGAGCCAACTGTGTGA | NM_003639.3 |
| 210 | IKBKG | CCAAACAGGAGGTGATCGA | NM_003639.3 |
| 211 | IKBKG | GGACAAGGCCTCTGTGAAA | NM_003639.3 |
| 212 | IKBKG | GGAAACTGGTGGAGAGACT | NM_003639.3 |
| 213 | IKBKG | GGGAGAAGCTGGCCGAGAA | NM_003639.3 |
| 214 | IKBKG | GCGAGGAGCTTCTGCATTT | NM_003639.3 |
| 215 | IKBKG | GGATCGAGGACATGAGGAA | NM_003639.3 |
| 216 | IKBKG | CGGCCAGGATCGAGGACAT | NM_003639.3 |
| 217 | IKBKG | GAGGAATGCAGCTGGAAGA | NM_003639.3 |
| 218 | IKBKG | TCGATAAGCTGAAGGAGGA | NM_003639.3 |
| 219 | IKBKG | TGGAGAAGCTCGATCTGAA | NM_003639.3 |
| 220 | IKBKG | AAACAGGAGGTGATCGATA | NM_003639.3 |
| 221 | IKBKG | CCAAACAGGAGGTGATCGA | NM_003639.3 |
| 222 | IKBKG | AGCAGATGGCTGAGGACAA | NM_003639.3 |
| 223 | IKBKG | AGAAGCTGGCCGAGAAGAA | NM_003639.3 |
| 224 | IKBKG | GCTTGGAGGCTGCCACTAA | NM_003639.3 |
| 225 | IKBKG | TGGCCAAACAGGAGGTGAT | NM_003639.3 |
| 226 | IKBKG | GGAAGAGCCAACTGTGTGA | NM_003639.3 |
| 227 | IKBKG | AACAGGAGGTGATCGATAA | NM_003639.3 |
| 228 | IKBKG | GAATGCAGCTGGAAGATCT | NM_003639.3 |
| 229 | IKBKG | AGAGGGAGGAGAAGGAGTT | NM_003639.3 |
| 230 | IKBKG | AGGCGGACTTCCAGGCTGA | NM_003639.3 |
| 231 | IKBKG | TGGAGCAGCTGCAGAGGGA | NM_003639.3 |
| 232 | IKBKG | GAGCAGCTGCAGAGGGAGT | NM_003639.3 |
| 233 | IKBKG | CGTACTGGGCGAAGAGTCT | NM_003639.3 |
| 234 | IKBKG | GGAGGAGAATCAAGAGCTC | NM_003639.3 |
| 235 | IKBKG | CCAGCCAGAGGGAGGAGAA | NM_003639.3 |
| 236 | IKBKG | GCCAGAGGGAGGAGAAGGA | NM_003639.3 |
| 237 | IKBKG | TGAAGAGGCAGAAGGAGCA | NM_003639.3 |
| 238 | IKBKG | TGGAAGATCTCAAACAGCA | NM_003639.3 |
| 239 | IKBKG | CTGAAGAGGCAGAAGGAGC | NM_003639.3 |
| 240 | IKBKG | AAGAATACGACAACCACAT | NM_003639.3 |
| 241 | IKBKG | GCAGAGGGAGTACAGCAAA | NM_003639.3 |
| 242 | IKBKG | CAGCAGATCAGGACGTACT | NM_003639.3 |
| 243 | IKBKG | AGTGAGCGGAAGCGAGGAA | NM_003639.3 |
| 244 | IKBKG | GAGCCAACTGTGTGAGATG | NM_003639.3 |
| 245 | IKBKG | CTGAAGGCCCAGGCGGATA | NM_003639.3 |
| 246 | IKBKG | CAGAGGGAGTACAGCAAAC | NM_003639.3 |
| 247 | IKBKG | CAGGAAACTGGTGGAGAGA | NM_003639.3 |
| 248 | IKBKG | TGAATAGGCACCTCTGGAA | NM_003639.3 |
| 249 | IKBKG | GAGGCTGCCACTAAGGAAT | NM_003639.3 |
| 250 | IKBKG | CCCAGTTGCAGGTGGCCTA | NM_003639.3 |
| 251 | IKBKG | GGGAGTACAGCAAACTGAA | NM_003639.3 |
| 252 | IKBKG | AGCGCTGCCTGGAGGAGAA | NM_003639.3 |
| 253 | IKBKG | AATCAAGAGCTCCGAGATG | NM_003639.3 |
| 254 | IKBKG | CGGCAGAGCAACCAGATTC | NM_003639.3 |
| 255 | IKBKG | AGAGATGCCAGCAGCAGAT | NM_003639.3 |
| 256 | IKBKG | AGGCCCAGGCGGATATCTA | NM_003639.3 |
| 257 | IKBKG | CTGCAGAGGGAGTACAGCA | NM_003639.3 |
| 258 | IKBKG | TGCAGAGGGAGTACAGCAA | NM_003639.3 |
| 259 | IKBKG | TGGACACCCTGCAGATACA | NM_003639.3 |
| 260 | IKBKG | GCACCTGCCTTCAGAACAG | NM_003639.3 |
| 261 | PLN | AAGTCCAATACCTCACTCGCT | NM_002667.3 |
| 262 | PLN | AAGCACGTCAAAAGCTACAGA | NM_002667.3 |
| 263 | PLN | AATTTCTGTCTCATCTTAA | NM_002667.3 |
| 264 | PLN | GGTCTTCACCAAGTATCAA | NM_002667.3 |
| 265 | PLN | GGCCATACTCTTACATAAT | NM_002667.3 |
| 266 | PLN | GGCAAGGAAAATAAAAGAT | NM_002667.3 |
| 267 | PLN | GCACGTCAAAAGCTACAGA | NM_002667.3 |
| 268 | PLN | GGCACTGTAGTGAATTATC | NM_002667.3 |
| 269 | PLN | GCTAGAGTTACCTAGCTTA | NM_002667.3 |
| 270 | PLN | AAGAAGAGCCTCAACCATT | NM_002667.3 |
| 271 | PLN | GTCAAAAGCTACAGAATCT | NM_002667.3 |
| 272 | PLN | CCTCACTCGCTCAGCTATA | NM_002667.3 |
| 273 | PLN | TCGCTCAGCTATAAGAAGA | NM_002667.3 |
| 274 | PLN | TGAAATGCCTCAACAAGCA | NM_002667.3 |
| 275 | PLN | AGCTATAAGAAGAGCCTCA | NM_002667.3 |
| 276 | PLN | TAATATGTCTCTTGCTGAT | NM_002667.3 |
| 277 | PLN | CCTCAACAAGCACGTCAAA | NM_002667.3 |
| 278 | PLN | GGAGAAAGTCCAATACCTC | NM_002667.3 |
| 279 | PLN | ACAAGCACGTCAAAAGCTA | NM_002667.3 |
| 280 | PLN | CGTCAAAAGCTACAGAATC | NM_002667.3 |
| 281 | PLN | ACGTCAAAAGCTACAGAAT | NM_002667.3 |
| 282 | PLN | GCTACAGAATCTATTTATC | NM_002667.3 |
| 283 | PLN | GCTATAAGAAGAGCCTCAA | NM_002667.3 |
| 284 | PLN | ATAAGAAGAGCCTCAACCA | NM_002667.3 |
| 285 | PLN | TAAGAAGAGCCTCAACCAT | NM_002667.3 |
| 286 | PLN | GCCTCAACAAGCACGTCAA | NM_002667.3 |
| 287 | PLN | AGAAGAGCCTCAACCATTG | NM_002667.3 |
| 288 | PLN | CAATTTCTGTCTCATCTTA | NM_002667.3 |
| 289 | PLN | TCAACAAGCACGTCAAAAG | NM_002667.3 |
| 290 | PLN | TTAATATGTCTCTTGCTGA | NM_002667.3 |
| 291 | PLN | GTCTCTTGCTGATCTGTAT | NM_002667.3 |
| 292 | PLN | CTTAATATGTCTCTTGCTG | NM_002667.3 |
| 293 | PLN | TCAGCTATAAGAAGAGCCT | NM_002667.3 |
| 294 | PLN | CCAATACCTCACTCGCTCA | NM_002667.3 |
| 295 | PLN | CAAAAGCTACAGAATCTAT | NM_002667.3 |
| 296 | PLN | CATCTTAATATGTCTCTTG | NM_002667.3 |
| 297 | PLN | AGAAAGTCCAATACCTCAC | NM_002667.3 |
| 298 | PLN | CTATAAGAAGAGCCTCAAC | NM_002667.3 |
| 299 | PLN | ACTCGCTCAGCTATAAGAA | NM_002667.3 |
| 300 | PLN | TCAAAAGCTACAGAATCTA | NM_002667.3 |
| 301 | PLN | TATCAATTTCTGTCTCATC | NM_002667.3 |
| 302 | PLN | TGTCTCTTGCTGATCTGTA | NM_002667.3 |
| 303 | PLN | AAGCACGTCAAAAGCTACA | NM_002667.3 |
| 304 | PLN | GCACGTCAAAAGCTACAGA | NM_002667.3 |
| 305 | PLN | TCAATTTCTGTCTCATCTT | NM_002667.3 |
| 306 | PLN | TGCCTCAACAAGCACGTCA | NM_002667.3 |
| 307 | PLN | TCCAATACCTCACTCGCTC | NM_002667.3 |
| 308 | PLN | CTCACTCGCTCAGCTATAA | NM_002667.3 |
| 309 | PLN | TACCTCACTCGCTCAGCTA | NM_002667.3 |
| 310 | PLN | CTGTCTCATCTTAATATGT | NM_002667.3 |
| 311 | PLN | CTCAACAAGCACGTCAAAA | NM_002667.3 |
| 312 | PLN | CAACAAGCACGTCAAAAGC | NM_002667.3 |
| 313 | PLN | TATAAGAAGAGCCTCAACC | NM_002667.3 |
| 314 | PLN | ATATGTCTCTTGCTGATCT | NM_002667.3 |
| 315 | PLN | CGCTCAGCTATAAGAAGAG | NM_002667.3 |
| 316 | PLN | ATCAATTTCTGTCTCATCT | NM_002667.3 |
| 317 | PLN | TGTCTCATCTTAATATGTC | NM_002667.3 |
| 318 | PLN | GTCTCATCTTAATATGTCT | NM_002667.3 |
| 319 | PLN | GTCCAATACCTCACTCGCT | NM_002667.3 |
| 320 | BIM | TGATGTAAGTTCTGAGTGT | NM_207002.2 |
| 321 | BIM | CCGAGAAGGTAGACAATTG | NM_207002.2 |
| 322 | BIM | TGACCGAGAAGGTAGACAA | NM_207002.2 |
| 323 | BIM | AAAGCAACCTTCTGATGTA | NM_207002.2 |
| 324 | BIM | ATGTAAGTTCTGAGTGTGA | NM_207002.2 |
| 325 | BIM | GTTCTGAGTGTGACCGAGA | NM_207002.2 |
| 326 | BIM | GACCGAGAAGGTAGACAAT | NM_207002.2 |
| 327 | BIM | GCACCCATGAGTTGTGACA | NM_207002.2 |
| 328 | BIM | CTACCTCCCTACAGACAGA | NM_207002.2 |
| 329 | BIM | CCCTACAGACAGAGCCACA | NM_207002.2 |
| 330 | BIM | GTGTGACCGAGAAGGTAGA | NM_207002.2 |
| 331 | BIM | AAGCAACCTTCTGATGTAA | NM_207002.2 |
| 332 | BIM | GAGTGTGACCGAGAAGGTA | NM_207002.2 |
| 333 | BIM | GCAACCTTCTGATGTAAGT | NM_207002.2 |
| 334 | BIM | TGACAAATCAACACAAACC | NM_207002.2 |
| 335 | BIM | GCAAAGCAACCTTCTGATG | NM_207002.2 |
| 336 | BIM | TGAGTGTGACCGAGAAGGT | NM_207002.2 |
| 337 | BIM | CAAAGCAACCTTCTGATGT | NM_207002.2 |
| 338 | BIM | GCCCAGCACCCATGAGTTG | NM_207002.2 |
| 339 | BIM | GTGACAAATCAACACAAAC | NM_207002.2 |
| 340 | BIM | CCTACAGACAGAGCCACAA | NM_207002.2 |
| 341 | BIM | AGTGTGACCGAGAAGGTAG | NM_207002.2 |
| 342 | BIM | GGCAAAGCAACCTTCTGAT | NM_207002.2 |
| 343 | BIM | CTCCCTACAGACAGAGCCA | NM_207002.2 |
| 344 | BIM | CTACAGACAGAGCCACAAG | NM_207002.2 |
| 345 | BIM | ACACAAACCCCAAGTCCTC | NM_207002.2 |
| 346 | BIM | GCCAGGCCTTCAACCACTA | NM_207002.2 |
| 347 | BIM | GAGAAGGTAGACAATTGCA | NM_207002.2 |
| 348 | BIM | AAATCAACACAAACCCCAA | NM_207002.2 |
| 349 | BIM | GGTAGACAATTGCAGCCTG | NM_207002.2 |
| 350 | BIM | CCCTACCTCCCTACAGACA | NM_207002.2 |
| 351 | BIM | GAGCCCAGCACCCATGAGT | NM_207002.2 |
| 352 | BIM | CTTGCCAGGCCTTCAACCA | NM_207002.2 |
| 353 | BIM | AGAAGGTAGACAATTGCAG | NM_207002.2 |
| 354 | BIM | GAAGGTAGACAATTGCAGC | NM_207002.2 |
| 355 | BIM | TCTGAGTGTGACCGAGAAG | NM_207002.2 |
| 356 | BIM | GTGACCGAGAAGGTAGACA | NM_207002.2 |
| 357 | BIM | AAGTTCTGAGTGTGACCGA | NM_207002.2 |
| 358 | BIM | TGCCAGGCCTTCAACCACT | NM_207002.2 |
| 359 | BIM | CGAGAAGGTAGACAATTGC | NM_207002.2 |
| 360 | BIM | GTAAGTTCTGAGTGTGACC | NM_207002.2 |
| 361 | BIM | TCTGATGTAAGTTCTGAGT | NM_207002.2 |
| 362 | BIM | GCCCCTACCTCCCTACAGA | NM_207002.2 |
| 363 | BIM | ACAGGAGCCCAGCACCCAT | NM_207002.2 |
| 364 | BIM | ACCGAGAAGGTAGACAATT | NM_207002.2 |
| 365 | BIM | AGCCCAGCACCCATGAGTT | NM_207002.2 |
| 366 | BIM | CAAATCAACACAAACCCCA | NM_207002.2 |
| 367 | BIM | CCTCCTTGCCAGGCCTTCA | NM_207002.2 |
| 368 | BIM | CTTCTGATGTAAGTTCTGA | NM_207002.2 |
| 369 | BIM | AGGTAGACAATTGCAGCCT | NM_207002.2 |

Further non-limiting examples of anti-htt siRNAs are provided in Table 4. It should be noted that in this table, the odd and even numbered siRNA strands are complementary to each other. It should be further noted that the siRNAs in table 4 are targeted to specific SNP variants of htt mRNA. The SNP nucleotide is shown in bold.

TABLE 4

| SEQ ID | |
|---|---|
| NO | SEQUENCE |
| 370 | 5′-aguggaugagggagcaggc-3′ |
| 371 | 5′-gccugcucccucauccacu-3′ |
| 372 | 5′-gcacacaguggaugaggga-3′ |
| 373 | 5′-ucccucauccacugugugc-3′ |
| 374 | 5′-ugaagugcacacaguggau-3′ |
| 375 | 5′-auccacugugugcacuuca-3′ |
| 376 | 5′-cacacaguggaugagggag-3′ |
| 377 | 5′-cucccucauccacugugug-3′ |
| 378 | 5′-gcacacaguagaugaggga-3′ |
| 379 | 5′-ucccucaucuacugugugc-3′ |
| 380 | 5′-ugaagugcacacaguagau-3′ |
| 381 | 5′-aucuacugugugcacuuca-3′ |
| 382 | 5′-aguagaugagggagcaggc-3′ |
| 383 | 5′-gccugcucccucaucuacu-3′ |
| 384 | 5′-cacacaguagaugagggag-3′ |
| 385 | 5′-cucccucaucuacugugug-3′ |
| 386 | 5′-ggcgcagacuuccaaaggc-3′ |
| 387 | 5′-gccuuuggaagucugcgcc-3′ |
| 388 | 5′-cacaagggcgcagacuucc-3′ |
| 389 | 5′-ggaagucugcgcccuugug-3′ |
| 390 | 5′-gcagggcacaagggcgcag-3′ |
| 391 | 5′-cugcgcccuugugcccugc-3′ |
| 392 | 5′-acaagggcgcagacuucca-3′ |
| 393 | 5′-uggaagucugcgcccuugu-3′ |
| 394 | 5′-cacaagggcacagacuucc-3′ |
| 395 | 5′-ggaagucugugcccuugug-3′ |
| 396 | 5′-gcagggcacaagggcacag-3′ |
| 397 | 5′-cugugcccuugugcccugc-3′ |
| 398 | 5′-ggcacagacuuccaaaggc-3′ |
| 399 | 5′-gccuuuggaagucugugcc-3′ |
| 400 | 5′-acaagggcacagacuucca-3′ |
| 401 | 5′-uggaagucugugcccuugu-3′ |
| 402 | 5′-caaugguacagcucuuccu-3′ |
| 403 | 5′-aggaagagcuguaccauug-3′ |
| 404 | 5′-caucccaaugguacagcuc-3′ |
| 405 | 5′-gagcuguaccauugggaug-3′ |
| 406 | 5′-ccaucccaaugguacagcu-3′ |
| 407 | 5′-agcuguaccauugggaugg-3′ |
| 408 | 5′-uuguggccaucccaauggu-3′ |
| 409 | 5′-accauugggauggccacaa-3′ |
| 410 | 5′-caacgguacagcucuuccu-3′ |
| 411 | 5′-aggaagagcuguaccguug-3′ |
| 412 | 5′-caucccaacgguacagcuc-3′ |
| 413 | 5′-gagcuguaccguugggaug-3′ |
| 414 | 5′-ccaucccaacgguacagcu-3′ |
| 415 | 5′-agcuguaccguugggaugg-3′ |
| 416 | 5′-uuguggccaucccaacggu-3′ |
| 417 | 5′-accguugggauggccacaa-3′ |
| 418 | 5′-gucggcaagcagagcuccc-3′ |
| 419 | 5′-gggagcucugcuugccgac-3′ |
| 420 | 5′-agccagucggcaagcagag-3′ |
| 421 | 5′-cucugcuugccgacuggcu-3′ |
| 422 | 5′-cagccagucggcaagcagagc-3′ |
| 423 | 5′-gcucugcuugccgacuggcug-3′ |
| 424 | 5′-gucucacagccagucggca-3′ |
| 425 | 5′-ugccgacuggcugugagac-3′ |
| 426 | 5′-gucagcaagcagagcuccc-3′ |
| 427 | 5′-gggagcucugcuugcugac-3′ |
| 428 | 5′-agccagucagcaagcagag-3′ |
| 429 | 5′-cucugcuugcugacuggcu-3′ |
| 430 | 5′-cagccagucagcaagcagagc-3′ |
| 431 | 5′-gcucugcuugcugacuggcug-3′ |
| 432 | 5′-gucucacagccagucagca-3′ |
| 433 | 5′-ugcugacuggcugugagac-5′ |
| 434 | 5′-cacauacauuagcucaaac-3′ |
| 435 | 5′-guuugagcuaauguaugug-3′ |
| 436 | 5′-cagcgucacauacauuagc-3′ |
| 437 | 5′-gcuaauguaugugacgcug-3′ |
| 438 | 5′-acauacauuagcucaaacu-3′ |
| 439 | 5′-aguuugagcuaauguaugu-3′ |
| 440 | 5′-cauuagcucaaacugguug-3′ |
| 441 | 5′-caaccaguuugagcuaaug-3′ |
| 442 | 5′-cacauacaucagcucaaac-3′ |
| 443 | 5′-guuugagcugauguaugug-3′ |
| 444 | 5′-cagcgucacauacaucagc-3′ |
| 445 | 5′-gcugauguaugugacgcug-3′ |
| 446 | 5′-acauacaucagcucaaacu-3′ |
| 447 | 5′-aguuugagcugauguaugu-3′ |
| 448 | 5′-caucagcucaaacugguug-3′ |
| 449 | 5′-caaccaguuugagcugaug-3′ |
| 450 | 5′-caacaucaaagcaucuuga-3′ |
| 451 | 5′-ucaagaugcuuugauguug-3′ |
| 452 | 5′-ccggccaacaucaaagcau-3′ |
| 453 | 5′-augcuuugauguuggccgg-3′ |
| 454 | 5′-uccggccaacaucaaagca-3′ |
| 455 | 5′-ugcuuugauguuggccgga-3′ |
| 456 | 5′-caaguuuccggccaacauc-3′ |
| 457 | 5′-gauguuggccggaaacuug-3′ |
| 458 | 5′-caaaaucaaagcaucuuga-3′ |
| 459 | 5′-ucaagaugcuuugauuuug-3′ |
| 460 | 5′-ccggccaaaaucaaagcau-3′ |
| 461 | 5′-augcuuugauuuuggccgg-3′ |
| 462 | 5′-uccggccaaaaucaaagca-3′ |
| 463 | 5′-ugcuuugauuuuggccgga-3′ |
| 464 | 5′-caaguuuccggccaaaauc-3′ |
| 465 | 5′-gauuuuggccggaaacuug-3′ |
| 466 | 5′-gaauuacuguccccaucuc-3′ |
| 467 | 5′-gagauggggacaguaauuc-3′ |
| 468 | 5′-gcguugaauuacugucccc-3′ |
| 469 | 5′-ggggacaguaauucaacgc-3′ |
| 470 | 5′-agcguugaauuacuguccc-3′ |
| 471 | 5′-gggacaguaauucaacgcu-3′ |
| 472 | 5′-ucuucuagcguugaauuac-3′ |
| 473 | 5′-guaauucaacgcuagaaga-3′ |
| 474 | 5′-gaaguacuguccccaucuc-3′ |
| 475 | 5′-gagauggggacaguacuuc-3′ |
| 476 | 5′-gcguugaaguacugucccc-3′ |
| 477 | 5′-ggggacaguacuucaacgc-3′ |
| 478 | 5′-agcguugaaguacuguccc-3′ |
| 479 | 5′-gggacaguacuucaacgcu-3′ |
| 480 | 5′-ucuucuagcguugaaguac-3′ |
| 481 | 5′-guacuucaacgcuagaaga-3′ |
| 482 | 5′-uagcguugaauuacugucc-3′ |
| 483 | 5′-ggacaguaauucaacgcua-3′ |
| 484 | 5′-uagcguugacuuacugucc-3′ |
| 485 | 5′-ggacaguaagucaacgcua-3′ |
| 486 | 5′-uagcguugaaguacugucc-3′ |
| 487 | 5′-ggacaguacuucaacgcua-3′ |
| 488 | 5′-uagcguugauguacugucc-3′ |
| 489 | 5′-ggacaguacaucaacgcua-3′ |
| 490 | 5′-cacauacauuggcucaaac-3′ |
| 491 | 5′-guuugagccaauguaugug-3′ |
| 492 | 5′-cacauacaucggcucaaac-3′ |
| 493 | 5′-guuugagccgauguaugug-3′ |

The exemplary targets provided above are suitable for neurodegenerative diseases (e.g., anti-htt siRNAs, anti-BACE-1 siRNAs, etc), heart diseases (e.g., anti-phospholamban siRNAs), treatment of inflammation and diseases associated with inflammation, such as schiatica (e.g., anti-TNF, anti-IL-1b siRNAs, anti-IKBKG, Anti-relA siRNAs).

The indication for use of anti-BIM siRNA is for polycystic kidney disease. Anti-BIM siRNA (specifically shRNA) has been shown to prevent polycystic kidney disease in mice. Reference: Cell Death and Differentiation (2005) 12, 831-833. doi:10.1038/sj.cdd.4401603. The above reference also defines the siRNA complementary to target sequence: TGATGTAAGTTCTGAGTGTG (SEQ ID NO: 494) as being most efficacious.

Of course, other diseases may also be targeted. A person of ordinary skill in the art possesses sufficient knowledge to select the appropriate gene for the selected disease or a condition.

In some embodiments, and depending on the problem in front of the practitioner, other RNA interference agents may be used. These methods are particularly suitable for delivering RNA to cells which are concentrated in one location within a patient (e.g., a subthalamic nucleus or a tumor, including, without limitations, benign and malignant tumors). For example, these methods may be used to treat localized infections or inflammations, autoimmune diseases, certain viral diseases, particularly those where the pathogen resides in certain cell types (e.g., herpes zoster, where the virus resides in trigeminal nucleus).

The siRNA molecules targeted to desired sequences can be designed based on criteria well known in the art (e.g., Elbashir et al., EMBO J. 20:6877 (2001)). For example, the target segment of the target mRNA preferably should begin with AA (most preferred), TA, GA, or CA; the GC ratio of the siRNA molecule preferably should be 45-55%; the siRNA molecule preferably should not contain three of the same nucleotides in a row; the siRNA molecule preferably should not contain seven mixed G/Cs in a row; the siRNA molecule preferably should comprise two nucleotide overhangs (preferably TT) at each 3′ terminus; the target segment preferably should be in the ORF region of the target mRNA and preferably should be at least 75 by after the initiation ATG and at least 75 by before the stop codon; and the target segment preferably should not contain more than 16-17 contiguous base pairs of homology to other coding sequences.

Based on some or all of these criteria, siRNA molecules targeted to desired sequences can be designed by one of skill in the art using the aforementioned criteria or other known criteria (e.g., Gilmore et al., J. Drug Targeting 12:315 (2004); Reynolds et al., Nature Biotechnol. 22:326 (2004); Ui-Tei et al., Nucleic Acids Res. 32:936 (2004)). Such criteria are available in various web-based program formats useful for designing and optimizing siRNA molecules (e.g., siDESIGN Center at Dharmacon; BLOCK-iT RNAi Designer at Invitrogen; siRNA Selector at Wistar Insitute; siRNA Selection Program at Whitehead Institute; siRNA Design at Integrated DNA Technologies; siRNA Target Finder at Ambion; and siRNA Target Finder at Genscript).

siRNA molecules targeted to desired sequences can be produced in vitro by annealing two complementary single-stranded RNA molecules together (one of which matches at least a portion of a desired nucleic acid sequence) (e.g., U.S. Pat. No. 6,506,559) or through the use of a short hairpin RNA (siRNA) molecule which folds back on itself to produce the requisite double-stranded portion (Yu et al., Proc. Natl. Acad. Sci. USA 99:6047 (2002)). Such single-stranded RNA molecules can be chemically synthesized (e.g., Elbashir et al., Nature 411:494 (2001)) or produced by in vitro transcription using DNA templates (e.g., Yu et al., Proc. Natl. Acad. Sci. USA 99:6047 (2002)). When chemically synthesized, chemical modifications can be introduced into the siRNA molecules to improve biological stability. Such modifications include phosphorothioate linkages, fluorine-derivatized nucleotides, deoxynucleotide overhangs, 2′-O-methylation, 2′-O-allylation, and locked nucleic acid (LNA) substitutions (Dorset and Tuschl, Nat. Rev. Drug Discov. 3:318 (2004); Gilmore et al., J. Drug Targeting 12:315 (2004)).

In other embodiments, the RNA of the instant invention is in a form of an aptamer, a spiegelmer, an antimir, or a combination thereof. Aptamers are nucleic acid structures which can bind to a target molecule in a way which is conceptually similar to antigen-antibody recognition. Aptamers may be selected by random library screening comprising, in different embodiments, more than 10¹⁵ different random sequences flanked by pre-determined sequences as to provide easily-identifiable PCR substrate.

Spiegelmers are subset of the aptamers which are created using L-nucleotides rather than naturally occurring D-nucleotides. Spiegelmers, therefore, are the mirror images of aptamers comprising D-nucleotides. The selection of spiegelmers is based on a simple logic: if an aptamer binds a mirror image of the target, then the mirror image of the aptamer (i.e., the spiegelmer) will bind the target itself. Thus, suitable spiegelmers may be selected by screening libraries of aptamers with mirror images of the targets.

Antimirs are short RNA molecules which bind to miRNAs naturally present in the cells. The methods of antimir selection are similar to those of siRNA selection, with the use of miRNA as a target.

In some embodiments, the RNA agents of the instant invention may be administered as vectorless RNA molecules.

In other embodiments, the dsRNA may be included within an RNA vector, which may be a single-stranded or a double-stranded RNA vector. The RNA strand (sense or antisense) which is to be incorporated into the vector, depends on whether the vector carries a positive or negative sense RNA. Suitable positive sense RNA vectors include, without limitations, Sindbis virus/replicons, Semliki Forest virus, Poliovirus, and Kunjin virus. Negative sense RNA viruses include, without limitations, Influenza virus, Rabies virus, Vesicular stomatitis virus, Respiratory syncytial virus, Sendai virus, SV5. The above represents a partial list of positive and negative sense RNA viruses, which have been genetically engineered to express foreign proteins. Some of the constructs give rise to infectious (attenuated) viruses, others form noninfectious replicons, which are restricted to replication (and expression of the foreign gene) in the transfected/infected cell. See, e.g, Palese, Proc Natl Acad Sci USA. 1998 October 27; 95(22): 12750-12752. A person of ordinary skill in the art would appreciate that the vectors which do not result in the infection is generally preferred. See, e.g., Li et al., J. of Virol., 2000, 74(14): 6564-6569.

The methods of preparing vectorless RNA, ssRNA viruses and dsRNA viruses are well-known in the art. For example, vectorless RNA agents may be produced by direct chemical synthesis. The RNA vectors may be produced according to the techniques described, for example, in U.S. Pat. Nos. 6,316,243, 6,544,785, 7,384,774, and 7,276,356.

The amount of the therapeutic of the present invention which will be effective in the treatment of a particular disorder or condition will depend on the nature of the disorder or condition, and can be determined by standard clinical techniques, well established in the administration of therapeutics. The precise dose to be employed in the formulation will also depend on the route of administration, and the seriousness of the disease or disorder, and should be decided according to the judgment of the practitioner and the patient's needs.

The concentration of the naked RNA may comprise, in different embodiments, up to 24 mg/ml, e.g., about 1 mg/ml, or about 2 mg/ml or about 3 mg/ml or about 4 mg/ml or about 5 mg/ml or about 6 mg/ml or about 7 mg/ml or about 8 mg/ml or about 9 mg/ml or about 10 mg/ml or about 11 mg/ml or about 12 mg/ml or about 13 mg/ml or about 14 mg/ml or about 15 mg/ml or about 15 mg/ml or about 16 mg/ml or about 17 mg/ml or about 18 mg/ml or about 19 mg/ml or about 20 mg/ml or about 21 mg/ml or about 22 mg/ml or about 23 mg/ml.

If the RNA is administered within a virus, the dose of the virus may be between about 10³ to 10¹⁵ infectious units of viral vector per microliter delivered in 1 to 3000 microliters of single injection volume. In different embodiments, the additional amounts of infections units of vector per micro liter would generally contain about 10⁴, 10⁵, 10⁶, 10⁷, 10⁸, 10⁹, 10¹⁰, 10¹¹, 10¹², 10¹³, 10¹⁴ infectious units of viral vector delivered in about 10, 50, 100, 200, 500, 1000, or 2000 microliters.

In another embodiment, the RNA of the instant invention may be delivered systemically (in addition to, or instead of the local delivery). Thus, it would pass the electrostimulated area and be taken up by the cells in the target area. Various modes of systemic delivery are known in the art including without limitations, intravascular delivery (including both intravenous and intraarterial delivery), intrathecal delivery, intraventricular delivery, intramuscular delivery, ingestion, intranasal delivery, intraocular delivery, intrapulmonary delivery, etc.

Particularly advantageous in these embodiments would be a formulation which protects the RNA from endo- and exoribonucleases naturally present in the patient's body.

In these embodiments, the composition would be administered in a dose which does not result in a therapeutically effective intake of the RNA by the cell in the non-stimulated area. Preferably, the RNA is administered in sufficiently low dose to avoid or minimize potential side effects, including, without limitation, immune response. At the same time, the dose should be sufficient to provide high enough concentration of the RNA so that the intake of RNA by the cells which are electrically stimulated according to the methods of the instant invention, would result in a therapeutically effective change in the amount of the target mRNA or the target protein.

Also envisioned is an external system that would pass a patient's blood through an electrical stimulator, with the addition of siRNA, in order to treat a septic condition. In one embodiment, anti-BIM siRNA is used as an inhibitor of sepsis. See, e.g., Schwulst et al., Shock. 2008 30(2):127-34, which states that “Treatment with Bim siRNA in vivo has the potential to be an effective therapy in the treatment of sepsis.”

Devices

In another broad aspect, the instant application provides devices suitable for the instant invention.

Generally, the devices of the instant invention comprise at least two electrodes and a catheter having an outlet opening for delivery of RNA. Generally, the outlet opening of the catheter is located between the electrodes. The catheter is fluidly connected to a reservoir comprising a composition comprising the RNA. Thus, for example, in one embodiment, the device comprises a plurality of electrodes; a catheter, comprising a wall and a cavity, wherein the members of said plurality of electrodes are disposed within or on the surface of said wall, a reservoir containing a composition comprising the nucleic acid sequence, said reservoir fluidly connected with said catheter, a pump operably connected to said reservoir, a processor operably connected to the members of said plurality of electrodes and adapted to receive electrical signals from said members and to deliver an electric field to said members. In one embodiment, the processor is adapted to actuate the pump after receiving a signal from the members of the plurality of electrodes. Further, considering that the strength of the electric field is the greatest on a straight line between the electrodes and predictably falls as the distance from that line increases, it is possible to provide a selected array of electrodes located as to provide the electric field of a pre-desired shape.

Other modifications of this basic device are possible. For example, the electrodes do not need to be integral with the catheter walls. Further, the electrodes may be configured in an external array which, in some embodiments would be placed onto the skin of the patient: e.g., an upper torso in the heart area, or a lower torso to provide a suitable electric field for the delivery of siRNA to kidneys, or the cranium for delivery of the RNA to the brain of the patient.

Considering that RNA is easily degradable, it may be advantageous in some embodiments to assure that the reservoir and the channels connecting the outlet opening and reservoir, and the composition are RNAse-free. This goal may be achieved, for example, by coating the surfaces with an RNAse inhibitor and/or by ultrafiltered water. See, e.g., Purad and Mabic, “RNase Undetectable In Water After Ultrafiltration” Biosci, Tech. 11: 26, 28 (2004). Another option of achieving the same result is preparing a DEPC-treated autoclaved water for the composition. During heat treatment, DEPC breaks down to ethanol and CO₂, and thus, the DEPC treated water is not toxic.

The devices may be further modified based on the organ which is to be treated (e.g., brain, heart, kidney, spinal cord). It should be further understood that if the use of the devices recited in the instant disclosure requires additional steps (e.g., the location of the predetermined area within a target organ), the methods of the instant invention may also entail optional additional steps, not necessarily limited to the structures of the devices recited in this application.

In one embodiment, the target area is within the central nervous system, e.g., the patient's brain. In this embodiment, the catheter may comprise an intracranial access catheter. The catheter will have a distal tip, which can be placed either in the parenchymal tissue of the brain or within a cerebral ventricle.

Generally, neurons affected with Huntington's disease reside in striatum, neurons affected with Alzheimer's disease reside in nucleus basalis of Meynart and the cerebral cortex, and neurons affected with Parkinson's disease reside in the substantia nigra. Thus, in different embodiments depending on the disease, the device delivers the therapies according to the methods of the instant invention to nucleus basalis of Meynart and the cerebral cortex, striatum, and/or the substantia nigra.

However, the methods of the instant invention are not limited to the target areas recited above. All substructures in central nervous system succeptible to DBS are also candidates for the treatment using the instant methods. For example, DBS targets subthalamic nucleus or globus pallidus in Parkinson's disease. There is a possibility of siRNA therapy in these regions too, e.g. to reduce neuron excitability (one of the mechanisms proposed by which DBS works) and enhance the effect of DBS.

Other applications of the instantly disclosed devices and methods to the treatment of nervous system include treatment of pain, sciatica, neuropathy, inflammation, etc. In these cases, the suitable targets may be inflammatory cytokines, NFKB, TNFX or voltage-gated sodium channels, SCN9A, and other compounds which are known to participate in generation, propagation, sustenance and/or amelioration of pain.

The target area may be located by many methods. For example, for some application, the targeted area may be located by stereotactical or gross anatomical atlases. In other embodiments, when the precise location of the targeted area is crucial, e.g., when the at least partially reversible gene therapy system is delivered into the brain of the patient, other mapping means may be used. Such mapping means include, without limitation, Positron Emission Tomography and Single Photon Emission Computed Tomography (PET and SPECT, respectively), pharmacological Magnetic Resonance Imaging (phMRI), functional MRI (fMRI), and contrast-enhanced computerized tomography (CT) scan.

In another embodiment, Computer-aided atlas-based functional neurosurgery methodology can be used to accurately and precisely inject the deoxyribonucleic acid of the present invention. Such methodologies permit three-dimensional display and real-time manipulation of cerebral structures. Neurosurgical planning with mutually preregistered multiple brain atlases in all three orthogonal orientations is therefore possible and permits increased accuracy of target definition for treatment injection or implantation, reduced time of the surgical procedure by decreasing the number of tracts, and facilitates planning of more sophisticated trajectories. See e.g. Nowinski W. L. et al., Computer-Aided Stereotactic Functional Neurosurgery Enhanced by the Use of the Multiple Brain Atlas Database, IEEE Trans Med Imaging 19(1); 62-69:2000.

Preferably, the pre-determined target area in the brain of the patient is determined on an individual basis, e.g., by real time image guidance, so that the neurosurgeon will see exactly where the catheter is being placed. Suitable systems exist for this particular embodiment, including, without limitation, STEALTH station developed by Surgical Navigation Technologies, a division of Medtronic. This tool incorporates preoperative images, including MRI, CT, and functional imaging studies into the computers in the operating room. A hand held probe linked to the computer can be used to point anywhere on the patients head or brain, with the corresponding area shown with great accuracy on a computer screen. Thus, there is no need to guess at the relationship between an area on or in the brain, inspected by sight and where that corresponds to the patient's preoperative images. Medtronic NT StealthStation® Treon™, further refines the computerized technologies of multi-dimensional imaging and navigation to enable neurosurgeons to precisely plan, re-plan, and visualize a procedure as it proceeds deep within the brain for treating neurological disorders in a living human patient.

As discussed above, the device may provide a plurality of electrodes positioned as to ensure the pre-determined shape of the electric field. In the embodiments where the brain is treated, the electrodes may be positioned as to achieve an electric field in the general shape of a pre-designed structure, e.g., in the shape of striatum, or caudate/putamen, thus further ensuring that the RNA of the instant invention is delivered to the desired brain structure. Similarly, if a ganglion (e.g., a dorsal root ganglion) is selected for the treatment, electrodes may be designed to ensure that the greatest electric field strength is within said ganglion.

In other embodiments, the location of the electrodes is verified after the insertion, e.g., through recording of the electric activity of the brain area surrounding the electrode. Optionally, the electrodes may be stimulated to invoke motor response from the patient to verify correct placement of catheter and then to apply the methods of the instant invention.

In other embodiments, e.g., where the heart is treated, the electrodes may also have the capability of sensing the pulses of naturally occurring electrical activity in the heart. The onset of such naturally occurring pulses would serve as a signal for delivering the coinciding bursts of the electrical activity according to the methods of the instant invention. Thus, in this embodiment, the electrodes would communicate the natural rhythms of the heart to the processor which, in turn, would initiate the delivery of the electrical field therapy to the heart.

In another aspect a kit is provided for practicing the methods according to any of the above-referenced embodiments. Briefly, the kit comprises a plurality of electrodes, a composition comprising RNA, and a processor adapted to actuate an electric stimulation by the members of said plurality of electrodes, to receive a signal from the members of said plurality of electrodes and, to actuate release of at least a portion of said composition comprising RNA within a predetermined time period after receiving said signal from the members of said plurality of electrodes.

Thus, the processor receives the signal from the members of the plurality of electrodes at a predetermined time, preferably, upon the beginning of the stimulation or upon the end of the stimulation. After a pre-determined time period, the processor actuates the release of at least the portion of the composition comprising RNA. The pre-determined period of time may range from, e.g., immediately after the processor receives signal, and up to 24 hours later. Thus, in different embodiments, the RNA is delivered to the cells at least 1 minute, at least 5 minutes, at least 30 minutes, at least 45 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least hours, at least 15 hours, at least 16 hours, at least 17 hours, at least 18 hours, at least 19 hours, at least 20 hours, at least 21 hours, at least 22 hours, at least 23 hours or 24 hours after the processor receives the signal from electrodes.

In selected embodiments, the composition is provided within a reservoir which, preferably, can be connected, or is connected, to a pump, said pump adapted to receive the signal from the processor, thus actuating the release of at least the portion (preferably, having pre-determined volume) of the RNA composition into the patient.

Optionally, a catheter is also provided. The catheter should be connectable to the reservoir, so that the signal from the processor to the pump will result in release of at least the portion of the RNA composition through a distal opening of the catheter.

In certain embodiments, it may be beneficial to combine the members of the plurality of electrodes and the catheter. For example, the electrodes may be positioned within or on the surface of a wall, or walls, of the catheter. In these embodiments, advantageously, one needs to perform only one placement. However, this requirement is not crucial, and multiple placements (e.g., for each electrode and for the catheter) may be performed. Separate catheter and electrodes may also be used if the desired location is such that spatial consideration prevents positioning of the combined device.

Specific embodiments according to the methods of the present invention will now be described in the following examples. The examples are illustrative only, and are not intended to limit the remainder of the disclosure in any way.

### Examples

### Example 1

### Electric Stimulation Leads to an Increased Uptake of RNA but Not of DNA or Small Molecules

HEK-293T cells were grown in Dulbecco's modified Eagle's medium (DMEM) supplemented with fetal bovine serum (10%), penicillin (100 IU/ml) and streptomycin (100 μg/ml) at 37° C. When the cells reached ˜70% confluency, they were electrically stimulated as follows:

DURATION 2 hours,

FREQUENCY 100 Hz,

FIELD STRENGTH 2 V/cm,

PULSE LENGTH 0.3 msec.

Immediately before stimulation, enhanced green fluorescent protein (GFP)-encoding supercoiled plasmid DNA (1 μg/ml) (pEGFP-C1, Clontech), rhodamine-conjugated 18-mer single stranded oligodeoxynucleotide (1 μg/ml) (Midland Certified Reagent Company), Alexa Fluor 660-conjugated short (530 base pairs) expression DNA cassettes (1 μg/ml), trypan blue dye (0.004%), or Alexa Fluor 555 hydrazide tris(triethylammonium salt) (1 μg/ml) (Invitrogen) were added to cells. Forty eight hours after stimulation, the cells were washed with sterile phosphate-buffered saline (pH 7.4) and nucleic acid uptake was estimated using fluorescence microscopy or dye uptake was estimated using bright-field microscopy.

Electrical stimulation did not increase the uptake of DNA or dye molecules in cells.

### Example 2

### Electrical Stimulation Does Not Affect Cell Viability

HEK-293 T cells were cultured as described in Example 1. Cells were treated with fluorophore-labeled RNA (siGLO Red, 1 μg/ml, Dharmacon). Electrical stimulation was performed as described in Example 1 with increasing electric field strength (0 V/cm; no stimulation group). Cell viability was accessed forty eight later using the MTT assay (performed per manufacturer's instructions; Trevigen Inc., Gaithersburg, Md.).

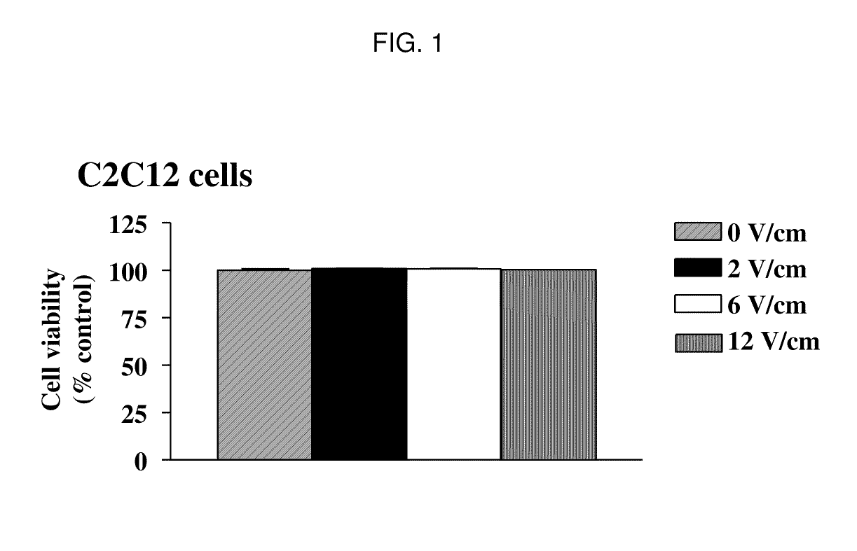

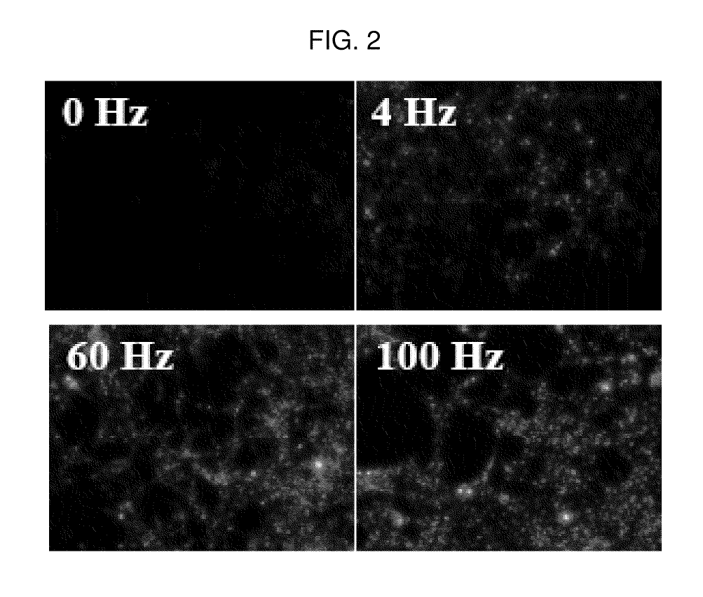

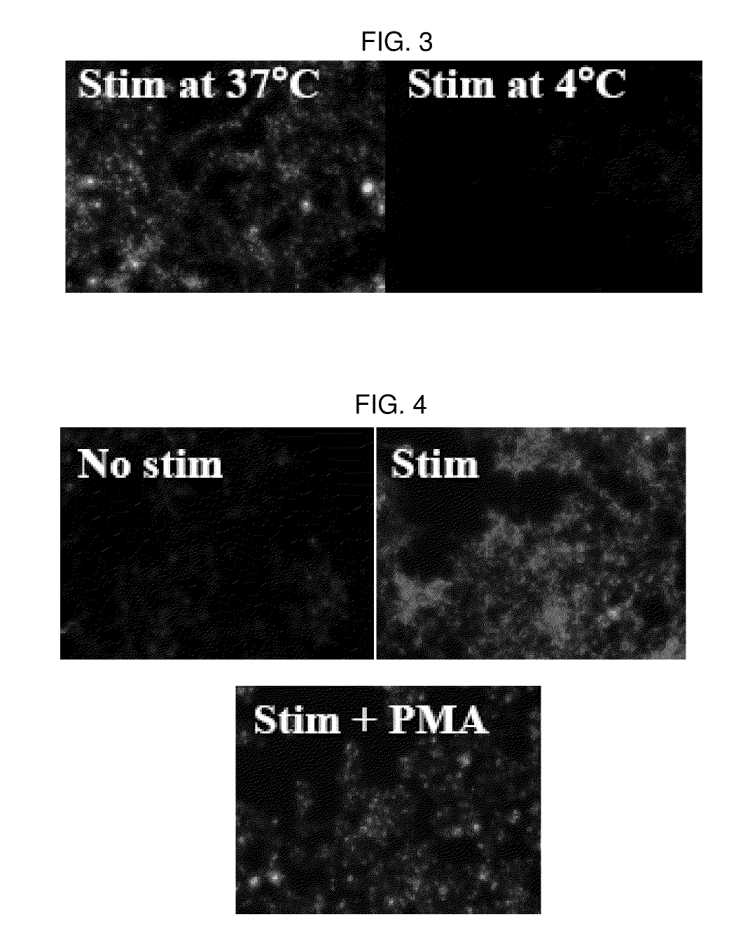

As shown in FIG. 1, stimulation with the electric fields of the chosen strength did not affect cell viability (FIG. 1).

### Example 3

### Uptake of RNA is Dependent on the Frequency of Electric Stimulation

HEK-293T cells were cultured as described in Example 1. Cells were treated with fluorophore-labeled RNA (siGLO Red, 1 μg/ml, Dharmacon). Electrical stimulation was performed as described in Example 1 with increasing frequencies (0 Hz; no stimulation group) as shown in Figure. After treatment, fluorophore uptake within cells was estimated using fluorescence microscopy.

This evidence suggests that even though low frequency stimulation (4 Hz) noticeably increased RNA uptake, stimulations with increased frequencies (60 Hz and 100 Hz) provide for a more efficient RNA uptake. These data also suggest that the electrical stimulation according to the methods of the instant invention does not disrupt membrane integrity of the stimulated cells.

### Example 4

### RNA Intake by Stimulated Cells is Due to a Cell-Based Process

HEK-293 cells were cultured as described above. Upon reaching ˜70% confluence, the cells were stimulated as described in Example 1 at 4° C. or 37° C. for 2 hours, followed by addition of 1 μg/ml of fluorophore-labeled RNA. It was found that the intake of RNA is temperature-dependent. Accordingly, it is likely that the electric stimulation triggered an active cell-based process for RNA intake.

### Example 5

### RNA Intake by Electrically Stimulated Cells is Attenuated by Inhibitors of Caveolae-Mediated Endocytosis

To further elucidate the mechanism of RNA uptake caused by electrical stimulation, the cells were cultured as in Example 1 and pre-treated for 15 minutes prior to the electrical stimulation with PMA (100 nm), a known inhibitor of caveolae-mediated endocytosis, and sucrose (100 nM), a known inhibitor of clathrin-mediated endocytosis.

As was expected from the prior experiments, electrically stimulated cells demonstrated a marked increase in RNA uptake, compared to non-stimulated cells, as measured by fluorescence microscopy. See FIG. 4A and 4B.

Pre-treatment of PMA decreased the RNA intake (FIG. 4C), suggesting the involvement of caveolae-mediated endocytosis in stimulation-enhanced uptake of RNA.

Pretreatment with sucrose also appeared to attenuate RNA intake, thus suggesting that clathrin mediated endocytosis is also involved in stimulation-enhanced uptake of RNA. However, due to cell loss in this group, it is impossible to draw any dispositive conclusions regarding the role of clathrin-mediated endocytosis.

### Example 6

### Electric Stimulation Improves Uptake of the RNA in Neuronal, Cardiac, and Kidney Cells in vitro

To further elucidate the parameters of the electric stimulation on the selected cell types, the inventors conducted experiments in multiple cell types in addition to HEK-293T cells. Specifically, C2C12 myoblasts and neuronal Neuro2a and BE(2)-C cells were studied.

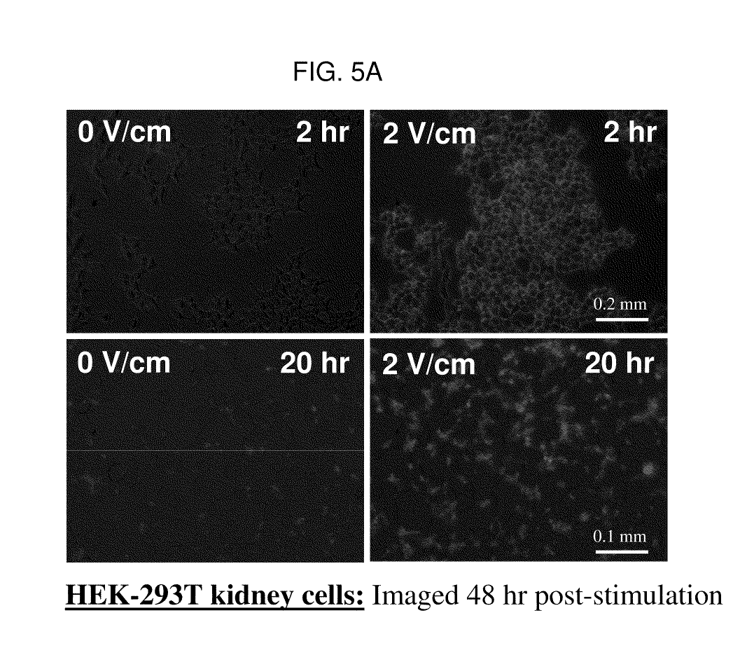

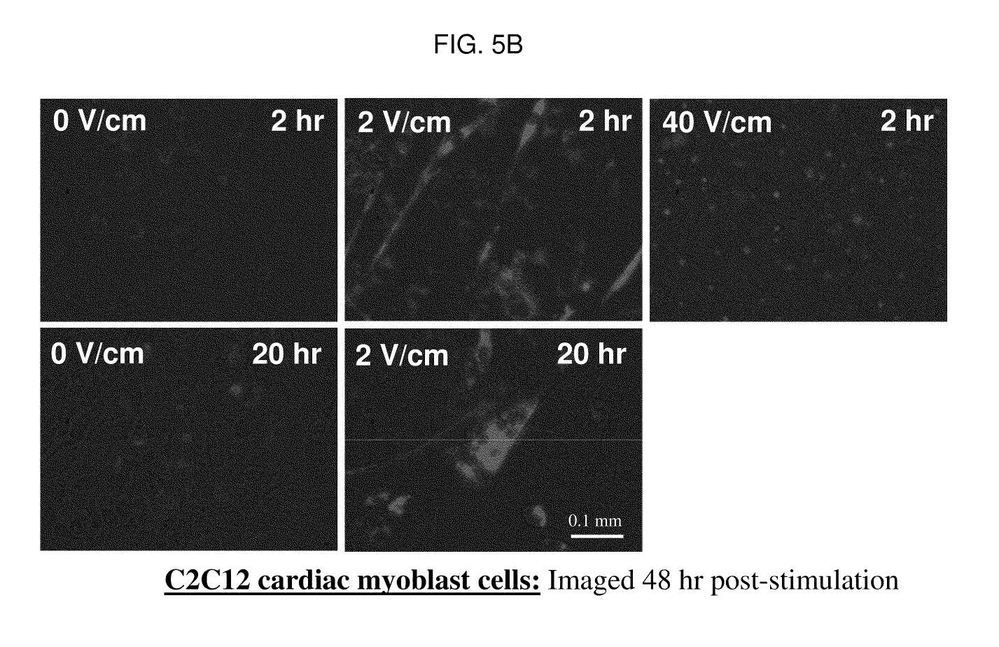

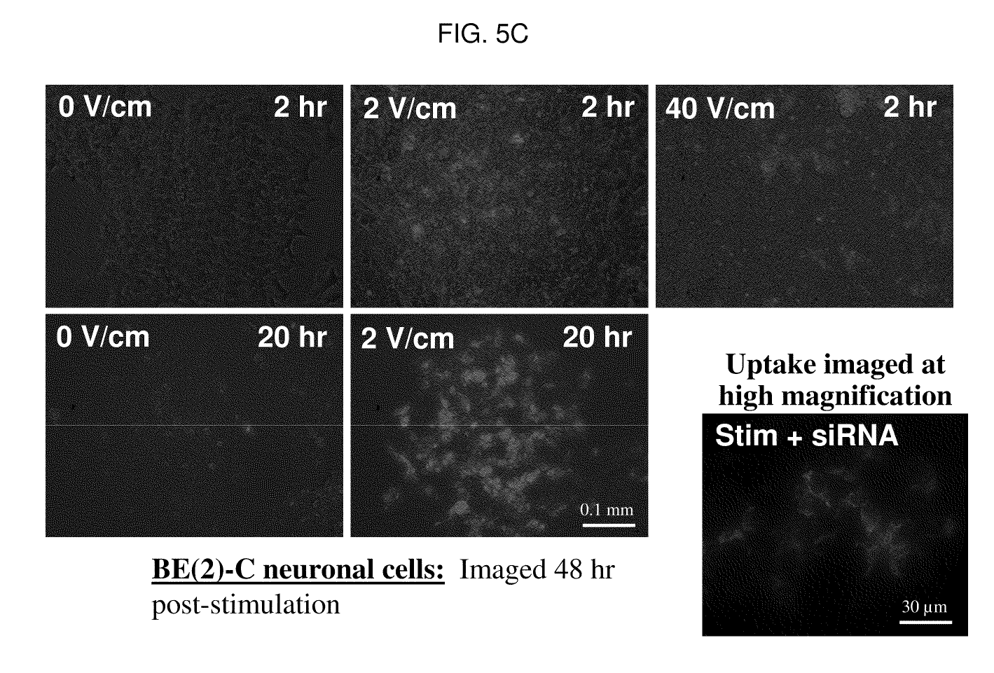

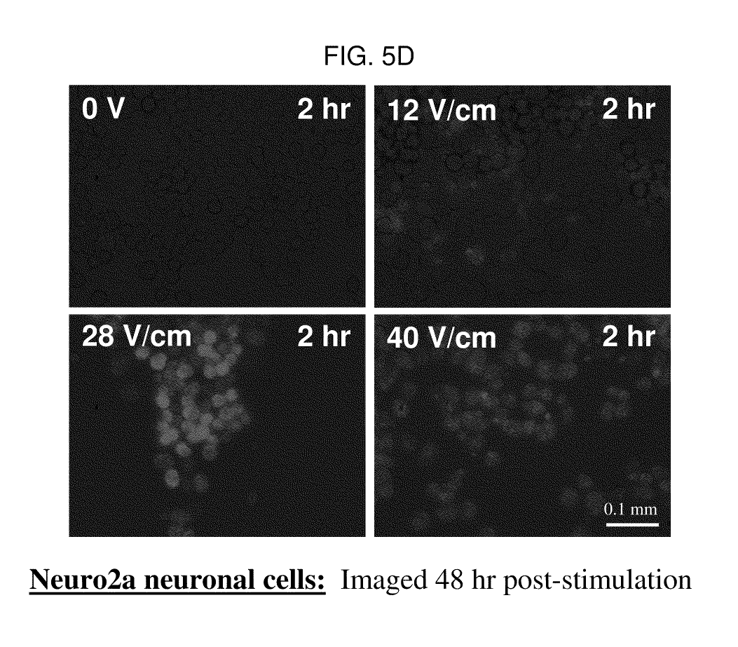

These cells were cultured similarly to HEK-293T cells (see Example 1), except that 1:1 ratio of DMEM and Earle's balanced salt solution were used instead of DMEM alone for Neuro2a cells. The results of RNA uptake, estimated by electron microscopy are shown in FIGS. 5A-5D, and are summarized in Table 5.

Electrical field strengths as low as 0.8 V/cm up to 12 V/cm applied for 2 hours at 100 Hz frequency and 0.3 msec pulse width enabled a substantial uptake of siRNA in the HEK-293T cells; the effect visibly reduced when the electrical field strength was reduced to 0.5 V/cm. In contrast, the same electrical parameters when applied for 20 hours were completely ineffective in delivering siRNA in these cells. Besides the duration of the applied electrical stimulation, siRNA uptake was also dependent on the frequency of electrical stimulation, such that frequencies <100 Hz (4 or 60 Hz) further reduced the degree of siRNA uptake in these cells.

The set of electrical parameters that enabled a robust uptake of siRNA in HEK-293T cells were distinct from the parameters required for achieving a similar outcome in C2C12 myoblasts. In the case of myoblasts, a burst-pulse protocol applied for longer duration (20 hours) of time was more effective in delivering siRNA, with 2 V/cm being the most effective and the effect slightly reducing with higher electrical field strengths (6 or 12 V/cm).

A longer duration of electrical stimulation was also more favorable for the delivery of siRNA in BE(2)-C human (predominantly dopaminergic) neuroblastoma cells. Testing the effects of electrical stimulation on siRNA uptake in yet another neuroblastoma cell-line, the mouse (predominantly cholinergic) Neuro2a cells, demonstrated a preference for a relatively higher electrical field strength (28 V/cm) applied for a relatively shorter duration of time (2 hours) to facilitate optimal siRNA uptake.

TABLE 5
Summary of RNA uptake efficiency caused by electric stimulations of different parameters.

| | siRNA | Electrical | | Time of | | | | | |
|---|---|---|---|---|---|---|---|---|---|
| Cell type | uptake | field strength | | stimulation | | Frequency | | Pulse width | |
| HEK-293T | ++ | 0.5 | V/cm | 2 | hours | 100 | Hz | 0.3 | msec |
| (kidney) | +++ | 0.8, 1, 2, 6 or 12 | V/cm | 2 | hours | 100 | Hz | 0.3 | msec |
| | − | 2, 6 or 12 | V/cm | 20 | hours | 100 | Hz | 0.3 | msec |
| | + | 2 | V/cm | 2 | hours | 4 | Hz | 0.3 | msec |
| | ++ | 2 | V/cm | 2 | hours | 60 | Hz | 0.3 | msec |
| | +++ | 2 | V/cm | 2 | hours | 100 | Hz | 0.2 or 0.45 | msec |
| C2C12 | + | 2, 6, or 12 | V/cm | 2 | hours, | 100 | Hz | 0.3 | msec |
| (cardiac) | | | | 20 | hours | | | | |
| | − | 2, 6 or 12 | V/cm | 2 | hours | 20 | Hz | 0.3 | msec |
| | | | | | | (2 bursts of 10 | | | |
| | | | | | | pulses, separated | | | |
| | | | | | | by 500 msec) | | | |
| | +++ | 2 | V/cm | 20 | hours | 20 | Hz | 0.3 | msec |
| | | | | | | (2 bursts of 10 | | | |
| | | | | | | pulses, separated | | | |
| | | | | | | by 500 msec) | | | |
| | ++ | 6 or 12 | V/cm | 20 | hours | 20 | Hz | 0.3 | msec |
| | | | | | | (2 bursts of 10 | | | |
| | | | | | | pulses, separated | | | |
| | | | | | | by 500 msec) | | | |
| BE(2)-C | + | 2, 6 or 12 | V/cm | 2 | hours | 100 | Hz | 0.3 | msec |
| (neuronal, | +++ | 2, 6 or 12 | V/cm | 20 | hours | 100 | Hz | 0.3 | msec |
| human) | | | | | | | | | |
| Neuro2a | − | 2, 6, 12 or 24 | V/cm | 0.5, 1, 2 or 20 | hours | 100 | Hz | 0.3 | msec |
| (neuronal, | +++ | 28 | V/cm | 2 | hours | 100 | Hz | 0.3 | msec |
| mouse) | | | | | | | | | |

Overall, these data demonstrate that the electric stimulation methods of the instant invention may be tailored to achieve optimal degree of siRNA uptake in specific cell types and at the same time the electric stimulation methods of the instant invention are sufficiently flexible to allow variability of different parameters without compromise in the effect of the RNA delivery. More importantly, the electrical stimulation parameters applied in this invention use much lower electrical field strengths (<40 V/cm) than used in electroporation (typically >200 V/cm) for delivering RNA in cells.

### Example 7

### The Increased Intake of RNA Persists for at Least 24 Hours After the electrical Stimulation

The temporal effect of the electrical stimulation on the intake of the RNA is discussed in this example 7.

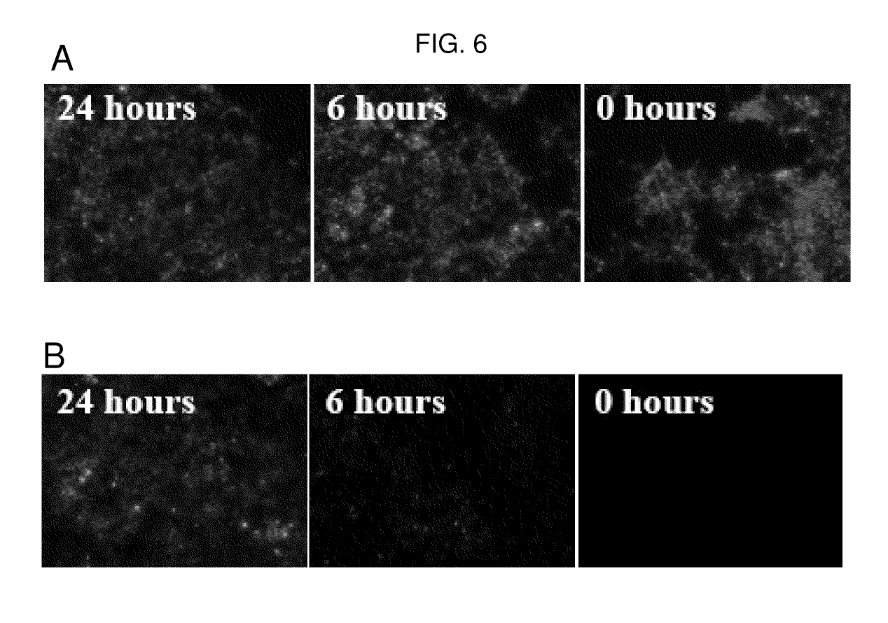

The cells were cultured and electrically stimulated as described in Example 1. In one group, the RNA was added immediately after the stimulation, or six hours after the stimulation, or 24 hours after the stimulation. The cells were imaged for the uptake at 48 hours after the electrical stimulation. In this group, electrical stimulation increased the RNA intake in all three subsets of cells, as shown in FIG. 6A., indicating that the siRNA uptake in cells was possible even if the siRNA was added after a 24 hour “lag period” following application of electrical stimulation.

In another group, the RNA was added before performing the stimulation according to Example 1, and then removed 24 hours, or 6 hours or immediately after performing the stimulation. The cells were imaged for the uptake at 48 hours after the electrical stimulation. In this group, only the subset of cells where the RNA was removed 24 hours after the stimulation (but not 6 hours after the stimulation or immediately after the stimulation) demonstrated an increased intake of the RNA, as shown in FIG. 6B.

The presence of a “lag period” between the application of electrical stimulation and siRNA administration in this invention is unexpected when compared with the requirements of using electroporation for delivery of nucleic acids. As reviewed by Escoffre J M et al. 2009 (Escoffre J M, Portet T, Wasungu L, Teissie J, Rols M P, What is (still not) known of the mechanism by which electroporation mediates gene transfer and expression in cells and tissues, Molecular Biotechnology, 2009, 41: 286-295) and Prud'homme G J et al. 2006 (Prud'homme G J, Glinka Y, Khan A S, Draghia-Akli R, Electroporation-enhanced nonviral gene transfer for the prevention or treatment of immunological, endocrine and neoplastic diseases, Current Gene Therapy, 2006, 6:243-273), electroporation-mediated intracellular delivery of nucleic acids relies on the simultaneous presence of the nucleic acid during application of the electroporation parameters that transiently allow the permeation of nucleic acids across the target cell's membrane.

The data in this disclosure demonstrate that the electrical stimulation used herein ensue long-term rather than transient effects, necessary to enable intracellular uptake of RNA; the long-term cellular changes could possibly be the alteration of gene expression pattern in target cells.

### Example 8

### RNA Delivered After the Stimulation Retains Its Biological Activity

Even though the electrical stimulation increases the intake of the RNA into the cells, the question remains whether this RNA is biologically active. This example proves that the uptaken RNA retains its biological activity.

HEK-293T cells were cultured in eight-well plates as in Example 1 and treated with 300 μsec impulses at 100 Hz and 2 V/cm for two hours. The anti-htt siRNA (GGAGTATTGTGGAACTTAT, SEQ ID NO: 5) at a concentration of 130 nM was added immediately before electrical stimulation.

In a control group the same siRNA at the same concentration was added to non-stimulated cells in a composition with TRANSIT-TKO® (Mirus Bio LLC, Madison, Wis.) according to manufacturer's instructions. Other control groups lacked one of the siRNA, TRANSIT-TKO® or the electric stimulation.

The cells were harvested 48 hours post-stimulation, and htt mRNA was quantified by qRT-PCR.

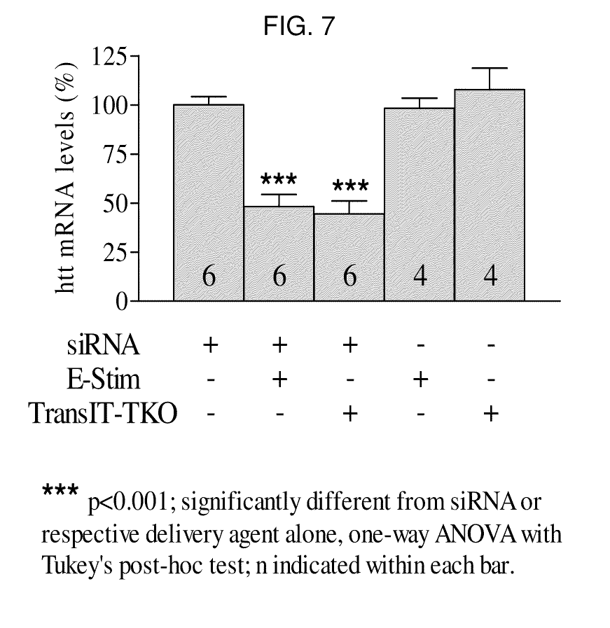

As shown in FIG. 7, the groups which received the siRNA in combination with TRANSIT-TKO® or with electrical stimulation exhibited significantly lower htt mRNA expression. In the group which received the siRNA in combination with the electrical stimulation, the htt mRNA level was about the same as in the group which received the siRNA in combination with TRANSIT-TKO®.

Accordingly, the siRNA delivered in conjunction with the electrical stimulation according to the instant invention retains its biological function.

### Example 9

### Electric Stimulation Improves Uptake of RNA in Neuronal Cells in vivo

The next question which needed to be resolved was whether the results obtained in vitro reasonably correlate with the results obtainable in vivo. Thus, in the remaining examples, the in vivo data are discussed.

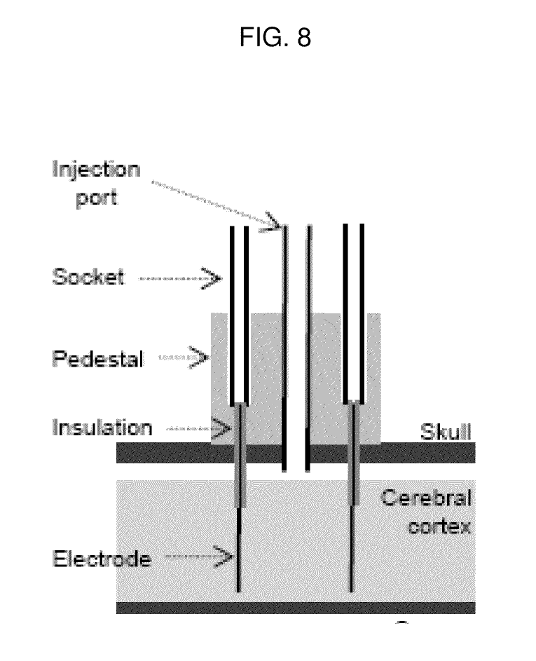

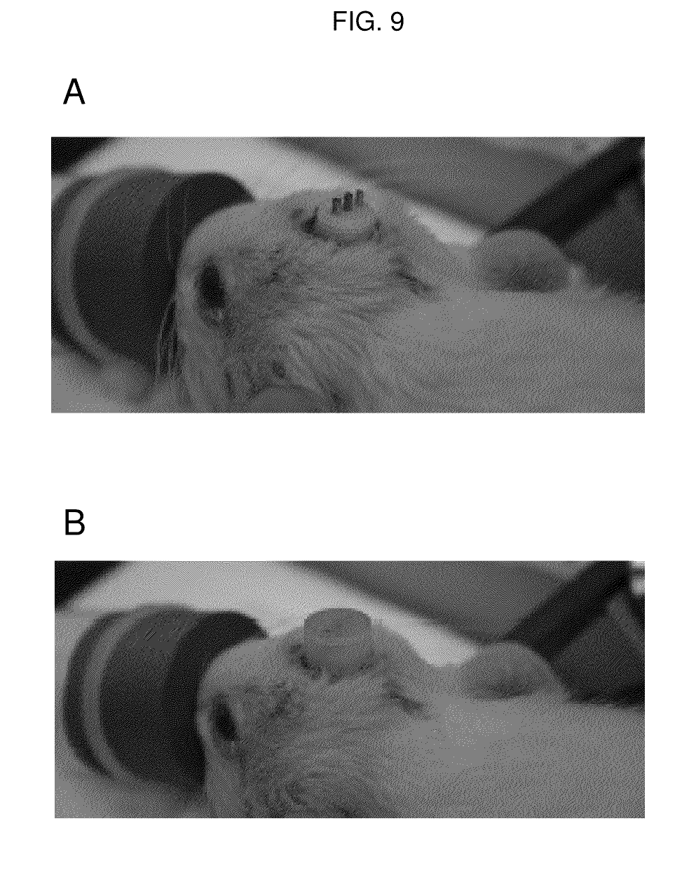

The device illustrated in FIG. 8 has been prepared and implanted into a brain of Sprague-Dawley rats weighing over 250 gm and over 8 weeks old. FIG. 9A. After the implantation of the device, rats were kept in cages (one rat per cage, at light-dark cycle 12 hr:12 hr, switching at 6 AM and 6 PM having unlimited access to food and water. After the surgery, the rats were left to recover for at least 1 hour in a cage with its floor maintained at 37° C. After the recovery, the rats were in their respective home cages and the device of the instant invention was capped as shown in FIG. 9B.

At the time of the experiment, and 22 hours after electrical stimulation, 4 μl of a solution containing siRNA (siGLO Red, purchased from Dharmacon, Inc. (Lafayette, Colo.)), labeled with fluorophore DY-547 in sterile saline, pH=7.4 at a concentration of 2.5 μg/μl, was injected using a stereotaxically guided syringe with a 33 gauge needle, at a rate of 0.25 pl/min.

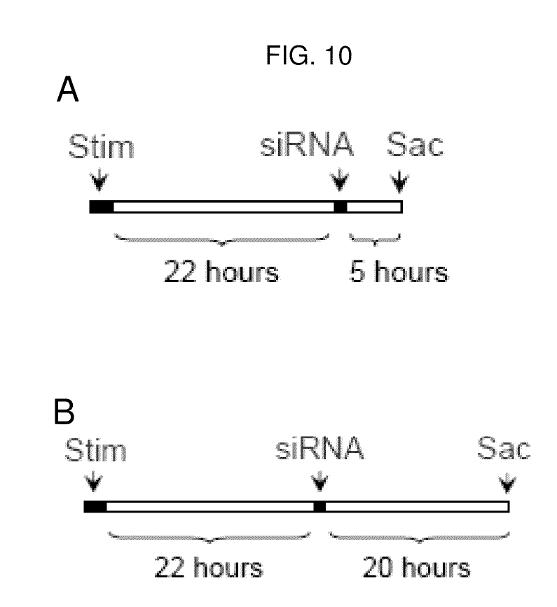

The rats were randomly split into the following groups:
a) Voltage groups: 0, 2, 6, 10 V/cm (4 groups);
b) Within each of the four groups, further treatment according to each of the protocols illustrated in FIGS. 10A and 10B.

Electrical stimulation was conducted with the rats in a conscious state. No gross behavioral abnormalities indicating pain or discomfort were observed during the treatment except that the rats when stimulated in the later half of their light cycle (but not in the first half of their light cycle) at 10 V/cm exhibited a mild periodic movement of their contralateral forelimb.

Three rats per group were used. The parameters of the electrical stimulation (other than voltage) were as follows:
a) Frequency: 100 Hz for each group;
b) Duration of stimulation: 2 hours for each group;
c) Pulse width: 0.3 msec for each group;
d) 4 μl of a solution containing siRNA (siGLO Red, purchased from Dharmacon, Inc. (Lafayette, Colo.)), labeled with fluorophore DY-547 in sterile saline, pH=7.4 at a concentration of 2.5 μg/μl was stereotaxically injected. The site was determined as the mid-point of the area between the uninsulated electrodes, with the following coordinates relative to Bregma: anterior-posterior: ˜0.7 mm; medial-lateral: ˜3.2 mm; dorso-ventral: ˜−2.0 mm.

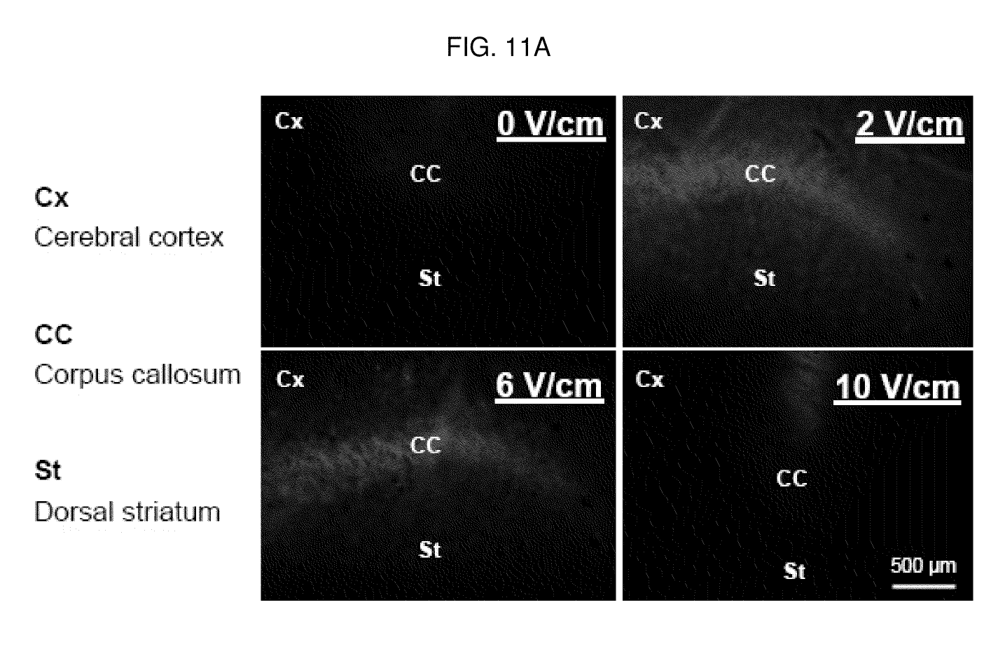

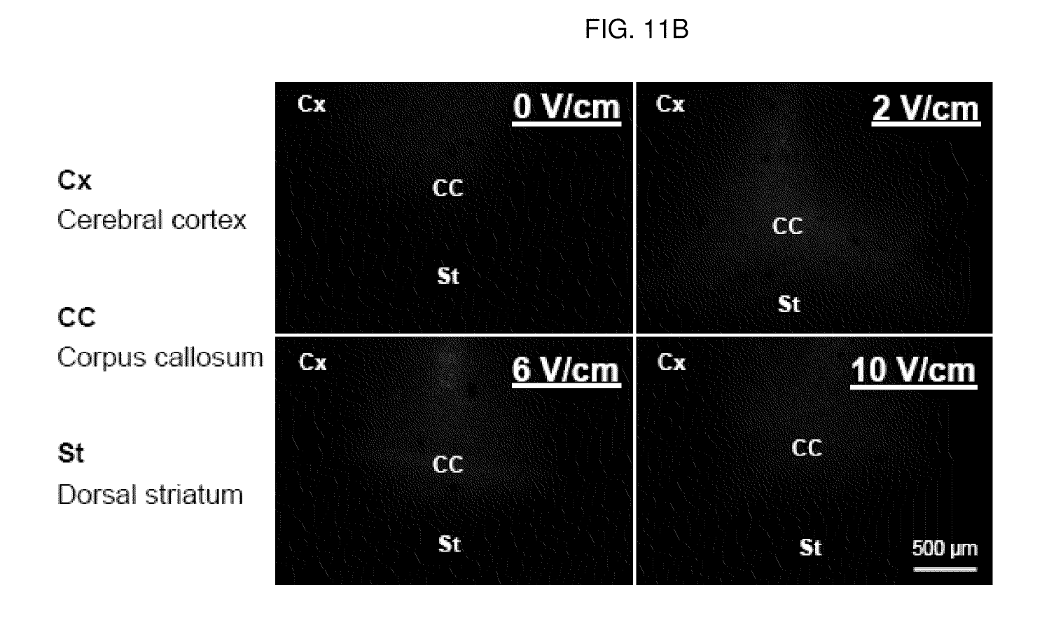

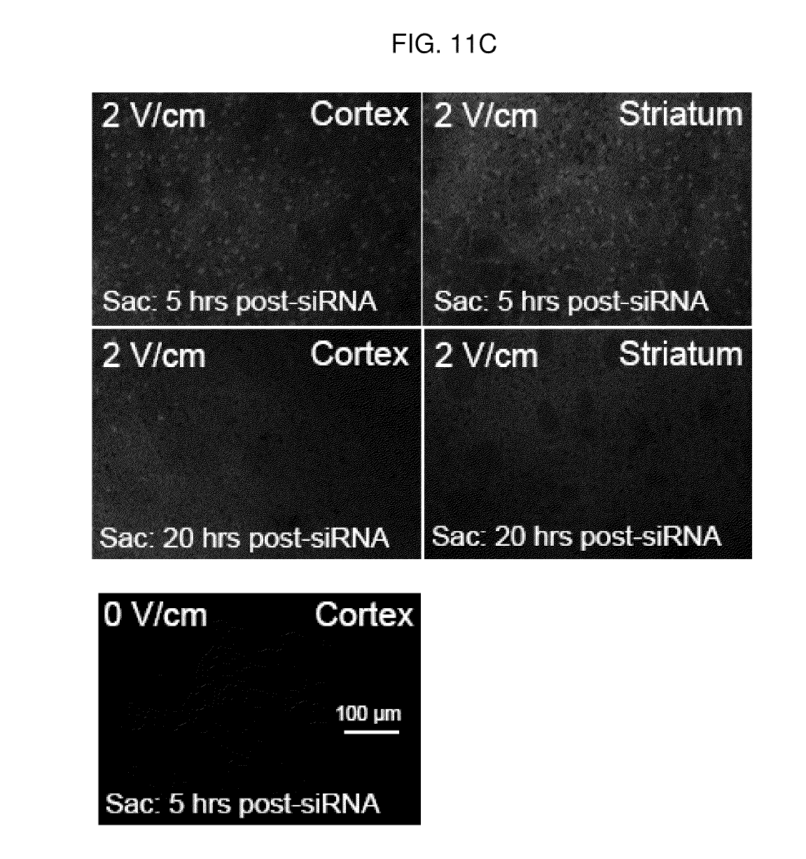

As shown in FIG. 11A, fluorescence imaging of brain sections revealed the intake of the RNA into the cells at five hours after the RNA injection at 2 V/cm, 6 V/cm, and, to a lesser extent, at 10 V/cm.

The intake of the RNA at 20 hours was not as well pronounced, but it was somewhat increased at 2 V/cm, 6 V/cm, and, to a lesser extent, 10 V/cm.

The greater magnification (20×), as shown in FIG. 11C revealed an intense fluorescence signal in both cortex and striatum at 5 hr post-siRNA group and 20 hr post siRNA groups who received electrical stimulation of 2 V/cm.

These data lead to several inferences. First, stimulation at 2-6 V/cm increases the uptake of siRNA that is injected for up to about 22 hours after electrical stimulation in the rat brain, and thus correlates with the results obtained in vitro. In other words, these in vivo results validate the in vitro findings by the inventors that application of low voltage and high frequency electrical stimulation facilitates the cellular uptake of siRNA that is administered after a “lag period” following stimulation.

Second, the facilitation of siRNA uptake was locally confined, dictated by the area of the electrical field. In the disclosed experiments, the local effects were as follows:
about 5 mm medio-laterally (inter-electrode distance was about 4.5 mm);
about 0.4 mm rostra-caudally (similar to the rostro-caudal distance between the electrodes); and
about 3 mm dorso-ventrally (electrode length was about 2 mm)

The electrical stimulation facilitated both the intracellular intake and extracellular distribution of the siRNA. The number of cells positive for the siRNA was increased at five hours post-siRNA injection, but only neuropil appeared positive at 20 hours post siRNA injection.

### Example 10

### Neurons and Oligodendrocytes are Brain Cells Susceptible to Pre-Treatment with Electric Stimulation

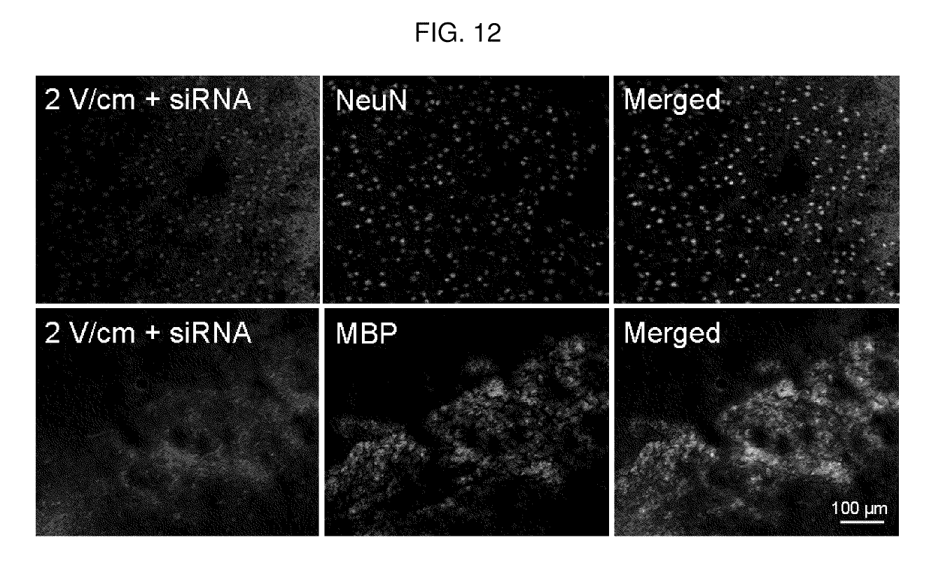

The inventors have further demonstrated that most of the cells visualized with the fluorophore were also visualized with NeuN and MBP (see FIG. 12), thus demonstrating that neurons and oligodendrocytes are the main cells types in the brain which are susceptible to electric stimulation treatment. Particularly, dopaminergic neurons (as determined by staining with DARPP32) also stained with fluorophore (data not shown).

Accordingly, neurons, particularly dopaminergic neurons, and oligodendrocytes are suitable targets for delivery of siRNA according to the instant methods.

### Example 11

### Electric Field Stimulation Improves RNA Delivery to Dorsal and Ventral Gray Horn Neurons

The facilitated cellular uptake of siRNA following electrical stimulation was evaluated in spinal cord tissue. The rat spared nerve injury (SNI) model was used in these studies. The SNI model is a peripheral injury model involving ligation (or ligation and transection) of the common peroneal and tibial branches of the sciatic nerve (Decosterd and Woolf, 2000, Spared nerve injury: an animal model of persistent peripheral neuropathic pain. Pain, 87: 149-58).

Spinal cord stimulation (SCS) was applied using an SCS lead similar to that described in Maeda et al., (2008; Low frequencies, but not high frequencies of bi-polar spinal cord stimulation reduce cutaneous and muscle hyperalgesia induced by nerve injury. Pain, 138: 143-52) following a post-surgical healing period of at least 14 days. This implant placed the SCS lead at the T10-T12 spinal level. The stimulation parameters tested included frequencies of 4, 60, 100 or 250 Hz at a pulse width of 250 μsec at a voltage of 85% of the motor threshold for minutes. Fluorescently labeled siGLO siRNA (Thermo Scientific) was continuously infused into the intrathecal (IT) space using an Alzet osmotic pump (Alzet Model 2001). siGLO siRNA was infused at 1 μl/hour at a concentration of 1 mg/ml; yielding a daily dose of 24 μg of siRNA. IT catheter and pump placement was achieved by advancing the distal end of a 28G rat intrathecal catheter (Alzet part #0007740) approximately 10 cm along the dorsal aspect of the subarachnoid space to terminate in the lumbar region at T10-T12, in the same proximity as the stimulation lead. The proximal end of the catheter, attached to the primed osmotic minipump, was implanted subcutaneously in the dorsal back via the same incision. On the day prior to IT catheter and pump implant, the animals were electrically stimulated using the parameters described above. The animals were then subsequently electrically stimulated on Days 3 and 5 post-catheter/pump implant. The animals were euthanized on Day 7 post-implant and the spinal cord was harvested for subsequent evaluation of the fluorescently tagged siGLO siRNA using fluorescent microscopy.

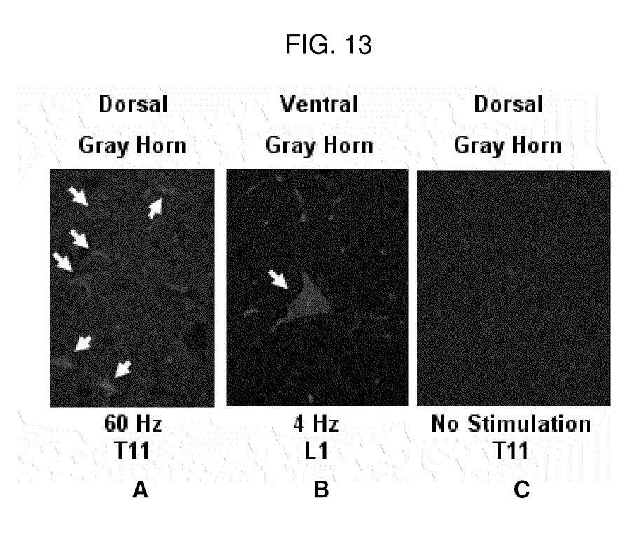

Analysis to date indicated enhanced uptake of siGLO siRNA within the gray matter of the spinal cord following electrical stimulation (FIG. 13A, B). Neuronal uptake of the siGLO siRNA is evident in proximity to the SCS lead. Cellular uptake of the siGLO siRNA was observed in both the dorsal and ventral gray horns of the cord. These positive neurons have been seen using stimulation frequencies of either 4 or 60 Hz. As shown in FIG. 13C, siGLO siRNA-positive neurons were not seen in spinal cords from animals did not receive electrical stimulation.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Other suitable modifications and adaptations to the methods and applications described herein are suitable and may be made without departing from the scope-of the invention or any embodiment thereof. While the invention has been described in connection with certain embodiments, it is not intended to limit the invention to the particular forms set forth, but on the contrary, it is intended to cover such alternatives, modifications and equivalents as may be included within the spirit and scope of the invention as defined by the following claims.

What is claimed is:

1. A method of increasing an uptake of an ribonucleic acid sequence by a cell, said cell being selected from the group consisting of cardiac cells, skeletal muscle cells, kidney cells, neurons and glial cells, the method comprising:
a) applying a plurality of pulses of an electric field to the cell for a time period between about two and about 24 hours;
b) within 24 hours of the step a), administering the ribonucleic acid sequence to the cell
wherein:
the electric field has strength of between about 0.5 V/cm and about 40 V/cm, calculated according to Formula I:
E=V/d
wherein in said formula E is the strength, V is Voltage and d is distance between electrodes.

2. The method of claim 1, wherein the frequency of said pulse is below 400 Hz.

3. The method of claim 1, wherein the duration of each member of the plurality of pulses is between about 100 μs and about 500 μs.

4. The method of claim 1 wherein the members of the plurality of pulses are uniform.

5. The method of claim 1 wherein the members of the plurality of pulses are not uniform.

6. The method of claim 1, wherein said ribonucleic acid sequence is a siRNA sequence, a shRNA sequence, an aptamer, a spiegelmer, an antimir, or a combination thereof.

7. The method of claim 6, wherein said ribonucleic acid sequence comprises at least one modified nucleotide.

8. The method of claim 6, wherein said ribonucleic acid sequence is a siRNA or a shRNA.

9. The method of claim 1, wherein said siRNA sequence or said shRNA sequence comprises a sense and an antisense strand, each having length between about 19 and about 30 nucleotides.

10. The method of claim 1, wherein
i) said cell is a neuron or a glial cell,
ii) the strength is between about 0.5 V/cm and about 10 V/cm, and
iii) the time period is between about 2 hours and about 22 hours.

11. The method of claim 10, wherein the strength is between about 1 V/cm and about 6 V/cm.

12. The method of claim 1 wherein
i) said cell is a neuron or a glial cell located within a nervous system of a patient,
ii) the patient is undergoing or is a suitable candidate for a deep brain stimulation or a spinal cord stimulation or a transcutaneous electric nerve stimulation, and
iii) the parameters of the electric stimulation are suitable for the deep brain stimulation, the spinal cord stimulation or the transcutaneous electric nerve stimulation.

13. The method of claim 10 further comprising steps of
locating a pre-determined area in a patient, said predetermined area being selected from a brain, a spinal cord, and a peripheral nerve of the patient; and
placing a plurality of electrodes into said pre-determined area prior to applying the plurality of pulses.

14. The method of claim 13, wherein the placement of the electrodes results in a pre-determined shape of the electric field.

15. The method of claim 10, wherein the nucleic acid is administered to the pre-determined area through a catheter.

16. The method of claim 15, further comprising a step of verifying the placement of the catheter.

17. The method of claim 10, further comprising a step of verification of the placement of the electrodes.

18. The method of claim 1, wherein the cell is a heart cell, and wherein the strength is between about 2 V/cm and about 12 V/cm, and wherein said the members of the plurality of pulses are administered as bursts having frequency of about ten pulses, said bursts separated by about 500 ms, and wherein the time period is about 20 hours.

19. The method of claim 1, further comprising the step of determining a heart rate of a patient, and wherein
i) the cell is a heart cell,
ii) the strength is between about 2 V/cm and about 12 V/cm,
iii) the time period is about 20 hours, and wherein said the members of the plurality of pulses, and
iv) the frequency of said bursts is about identical to the heart rate of the patient.

20. The method of claim 19, wherein an onset of a heart beat initiates the application of the burst of pulses of the electric field.

21. The method of claim 18, wherein the siRNA or the shRNA inhibits expression of phospholamban.

22. The method of claim 1, wherein the cell is a kidney cell, and wherein the strength is between about 0.5 and about 40 V/cm and the time period is between about one and about 12 hours.

23. The method of claim 22, wherein the time period is about two hours.

24. The method of claim 22, wherein the siRNA or the shRNA inhibits expression of BIM.

25. The method of claim 1, wherein the ribonucleic acid sequence is administered in a composition comprising an imaging agent, the method further comprising: determining a distribution of the composition.

26. The method of claim 18, wherein
i) said plurality of pulses of an electric field are applied to a pre-determined area within a heart or a kidney of a patient through a plurality of electrodes,
ii) said plurality of electrodes are positioned relative to each other as to provide an electric field of a pre-determined shape.

27. The method of claim 1, wherein step (b) is initiated at least 1 minute, at least 5 minutes, at least 30 minutes, at least 45 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least hours, at least 21 hours, at least 22 hours, at least 23 hours or 24 hours after the initiation of step (a).

28. A device for delivering a nucleic acid sequence to a cell within a patient, the device comprising:
a plurality of electrodes;
a catheter, the catheter comprising a wall and a cavity;
a reservoir containing a composition comprising the RNA, said reservoir fluidly connected with said catheter, wherein said composition is RNAse-free;
a pump operably connected to said reservoir;
a processor operably connected to the members of said plurality of electrodes and adapted to receive electrical signals from said members and to deliver an electric field to said members.

29. The device of claim 28, wherein said composition further comprises an imaging agent.

30. The device of claim 28, wherein said nucleic acid sequence comprises or encodes a shRNA or a siRNA, said shRNA or said siRNA comprising an antisense having a length between about 19 and about 30 nucleotides.

31. The device of claim 28, wherein the processor is adapted to actuate the pump after receiving a signal from the members of the plurality of electrodes.

32. The device of claim 28 wherein the members of the plurality of electrodes are positioned to achieve a pre-determined shape of the electric field.

33. The device of claim 32, wherein said pre-determined shape is designed to substantially correspond to a distribution of the composition comprising the nucleic acid sequence in a pre-determined location within a patient.

34. The device of claim 28, wherein the reservoir and the catheter are RNAse-free.

35. The device of claim 28, wherein the composition comprises an RNAse inhibitor.

36. The device of claim 28, wherein the plurality of electrodes are configured to deliver a deep brain stimulation, a spinal cord stimulation or a transcutaneous electric nerve stimulation to the patient.

37. The device of claim 36, wherein the deep brain stimulation has the field strength of about 2 V/cm to about 4 V/cm, the frequency of about 100 Hz to about 185 Hz, and pulse width of between about 90 μs and about 180 μs.

38. The device of claim 32, wherein
a) the members of the plurality of electrodes sense the rhythmic electric activity of a predetermined organ within the patient's body, said pre-determined organ comprising the cell,
b) the onset of said rhythmic electric activity initiates delivery of an electric stimulus to the pre-determined organ, the electric stimulus comprising a plurality of bursts, having an electric field strength between about 2 V/cm and about 12 V/cm, and the frequency about identical to the frequency of said rhythms of electric activity.

39. The device according to claim 28, wherein the members of said plurality of electrodes are disposed within or on the surface of said wall.

40. A medical system comprising:
a plurality of electrodes;
a catheter, the catheter comprising a wall and a cavity, wherein the members of said plurality of electrodes are disposed within or on the surface of said wall;
a reservoir containing a composition comprising the RNA, said reservoir fluidly connected with said catheter;
a pump operably connected to said reservoir;
a processor operably connected to the members of said plurality of electrodes and adapted to receive electrical signals from said members and to deliver an electric field to said members; and
a patient-specific intraoperative image-guided mapping means.

41. A kit comprising:
a) a plurality of electrodes;
b) a composition comprising RNA;
c) a processor adapted to:
i) actuate an electric stimulation by the members of said plurality of electrodes;
ii) receive a signal from the members of said plurality of electrodes and,
iii) within a predetermined time period after receiving said signal from the members of said plurality of electrodes, to actuate release of at least a portion of said composition comprising RNA.

42. The kit of claim 41, wherein said composition containing ribonucleic acid sequence is within a reservoir operably connectable to a pump, and wherein the processor actuates release of at least a portion of said composition comprising ribonucleic acid sequence by sending a signal to the pump.

43. The kit of claim 41, wherein the processor is programmed actuate said electric stimulation of no greater than about 40 V/cm.

44. The kit of claim 41, wherein said predetermined period is between about one hour and about 24 hours.

45. The kit of claim 41, further comprising a means for positioning the members of said plurality of electrodes and said catheter in a desired location, wherein said location is electrically stimulatable by members of said plurality of electrodes.

46. The kit of claim 41, further comprising a catheter fluidly connectable to the reservoir, said catheter having a wall and a distal opening, wherein at least the portion of said composition comprising ribonucleic acid sequence is releasable through said distal opening.

47. The kit of claim 46, wherein the members of said plurality of electrodes are disposed within or on the surface of a wall of the catheter.

48. The kit of claim 46, comprising a means for positioning the members of said plurality of electrodes and said catheter in a pre-determined location, wherein said pre-determined location is electrically stimulatable by members of said plurality of electrodes and wherein at least the portion of composition comprising RNA reaches the pre-determined location after being released from the distal opening.

49. The kit of claim 41, wherein said release of at least a portion of said composition comprising RNA is actuated at least 1 minute, at least 5 minutes, at least 30 minutes, at least 45 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least hours, at least 15 hours, at least 16 hours, at least 17 hours, at least 18 hours, at least 19 hours, at least 20 hours, at least 21 hours, at least 22 hours, at least 23 hours or 24 hours after actuating of the electric stimulation by the members of said plurality of electrodes.
